(19) United States
(12) Patent Application Publication
(10) Pub. No.: US 2026/0226184 A1
(43) Pub. Date: Aug. 6, 2026

(54) DEVELOPING A CAR-ENGAGER PLATFORM TO ENHANCE THE FUNCTIONALITY AND/OR PERSISTENCE OF CAR T CELLS

(71) Applicant: DANA-FARBER CANCER INSTITUTE, INC., Boston, MA (US)

(72) Inventors: Mohammad RASHIDIAN, Boston, MA (US); Taha RAKHSHANDEHROO, Boston, MA (US)

(73) Assignee: DANA-FARBER CANCER INSTITUTE, INC., Boston, MA (US)

(21) Appl. No.: 19/150,427

(22) PCT Filed: Jan. 26, 2024
(86) PCT No.: PCT/US2024/013091

§ 371 Date: Jul. 23, 2025

Related U.S. Application Data
(60) Provisional application No. 63/441,253, filed on Jan. 26, 2023.

(51) Int. Cl. C07K 16/28; A61K 31/454; A61K 31/69; A61K 35/28; A61K 40/11; A61K 40/31; A61K 40/42; A61P 35/00

(52) U.S. Cl. CPC C07K 16/2878; A61K 31/454; A61K 31/69; A61K 35/28; A61K 40/11; A61K 40/31; A61K 40/4202; A61K 40/4211; A61P 35/00; C07K 16/2803; C07K 2317/622; C07K 2317/77; C07K 2317/92

(57) ABSTRACT

Disclosed are chimeric antigen receptor (CAR)-engagers and proteinaceous entities and dimers containing an ectodomain of an antigen present on a cancer cell and a first immune cell effector domain, and uses thereof in concert with CAR-immune cells to treat cancer.

### CROSS-REFERENCE TO RELATED APPLICATIONS

This application claims the benefit of priority under 35 U.S.C. § 119(e) to U.S. Provisional Application No. 63/441,253, filed Jan. 26, 2023, which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 22, 2024, is named 52095_774001WO_ST.xml and is 109 KB bytes in size.

### BACKGROUND OF THE DISCLOSURE

Chimeric antigen receptor (CAR) expressing T cells have revolutionized the treatment of blood-borne malignancies and have shown promising results in the treatment of hematopoietic cancers. Six CAR T cell therapies targeting two antigens, CD19 and BCMA, are currently FDA-approved. CD19 is a B-cell co-receptor expressed on B cells and a wide variety of blood-borne malignancies. CD19 CAR T cells were initially approved for the treatment of acute lymphoblastic leukemia (ALL) and have subsequently been approved for Burkitt's Lymphoma and Mantle Cell Lymphoma. BCMA is a receptor expressed on the surface of B-cell lineage cells and a major marker of multiple myeloma (MM). MM is associated with an uncontrollable expansion of plasma cells in the bone marrow, which can progress to extra-medullary lesions forming elsewhere in the body. BCMA CAR T cell therapy has shown great promise against MM with studies showing an overall response rate of 80% even in patients with extra-medullary lesions (Gagelmann et al., Eur. J. Haematol. 104(4):318-327 (2020)).

However, challenges persist with CAR T cell therapy. A recent meta-analysis of 22 CAR T cell clinical studies highlighted its ineffectiveness in solid tumors with a poor average overall response rate of 9% (Hou et al., Dis. Markers 2019:3425291 (2019)). The duration of response, even in hematological cancers remains a challenge, with almost all BCMA CAR-treated MM patients ultimately relapsing (Gagelmann et al., Eur. J. Haematol. 104(4):318-327 (2020); Roex et al., J. Hematol. Oncol. 13(1):164 (2020); Raje et al., N. Engl. J. Med. 380(18):1726-1737 (2019)). Treatments can have severe side effects including cytokine release syndrome (CRS) and neurotoxicity.

CAR T cells need to home to the tumor location, expand, and persist in circulation, at least until they neutralize and kill the last remaining cancer cells. Therefore, approaches to modulate the activity of CAR T cells in a controlled way are critically needed.

### SUMMARY OF THE DISCLOSURE

The presently disclosed chimeric antigen receptor (CAR)-engagers and methods of use thereof, are expected to address the above needs. The CAR-engagers augment CAR immune cell functionality and persistence in vivo. They may also reduce the cellular dose needed for CAR immune cell therapy, which may result in reduced adverse side effects (e.g., cytokine release syndrome) caused by the larger doses typically used in the clinic and therefore, the CAR-engagers are also referred herein as CAR-enhancers. Further, since CAR-engager binding to CAR is reversible, the CAR engager does not induce immune synapse formation of CAR on the CAR immune cell surface. Therefore, CAR-engagers do not block CAR-mediated killing of cancer cells. CAR immune cells often do not persist in the body during minimal residual disease (MRD), which, as known in the art, is associated with limited cancer antigens. The disclosed CAR-engagers may support persistence, proliferation, and efficacy of CAR T cells during states of MRD.

A first aspect of the present disclosure is directed to a chimeric antigen receptor (CAR)-engager that contains an ectodomain of an antigen present on a cancer cell connected to an immune cell effector domain. The connection between the ectodomain-containing portion and the immune effector domain may be peptidic or non-peptidic (covalent), such that the CAR-engager may be a continuous protein or polypeptide, or a moiety that contains two proteinaceous entities connected by a covalent bond. The ectodomain of the CAR-engager is designed so as to bind the extracellular domain of a CAR that targets the ectodomain of the antigen on a cancer cell. In some embodiments, the binding between CAR and cancer antigen is direct. In some embodiments, the binding between CAR and cancer antigen is indirect. In these embodiments, the CAR-engager ectodomain is a cancer irrelevant antigen present in a protein therapeutic. The cancer irrelevant antigen in the protein therapeutic and CAR-engager ectodomain binds a CAR. The protein therapeutic serves to redirect the CAR immune cell from the cancer irrelevant antigen to a cancer antigen through a cancer antigen binding domain. Such CAR immune cells are referred to as universal CAR or binary activated CAR (BAT-CAR). A representative cancer irrelevant antigen is the small molecule fluorescein isothiocyanate (FITC) to target universal anti-FITC CAR immune cells.

Another aspect of the present disclosure is directed to a heterodimeric CAR-engager, containing a first entity containing an ectodomain of an antigen present on a cancer cell connected to first dimerization domain, and a second entity containing a first immune cell effector domain connected to a second dimerization domain, wherein the first and the second dimerization domains bind to form a heterodimeric CAR-engager. In embodiments wherein the connections are peptide bonds, the first and second entities are referred to as first and second proteins or polypeptides.

Yet other aspects of the present disclosure are directed to a nucleic acid that encodes a CAR-engager protein, a nucleic acid that encodes a first entity of the heterodimeric CAR-engager protein, and a nucleic acid that encodes the second entity of the heterodimeric CAR-engager protein.

Yet other aspects of the present disclosure are directed to a vector containing the nucleic acid encoding the CAR-engager protein, and a vector containing the first and/or second proteins of the heterodimeric CAR-engager.

Yet another aspect of the present disclosure is directed to a cell transformed with the vector(s).

Another aspect of the present disclosure is directed to a pharmaceutical composition that contains the CAR-engager and a pharmaceutically acceptable carrier.

Another aspect of the present disclosure is directed to a method of making a CAR-engager protein. The method entails culturing a cell transformed with the vector containing the nucleic acid encoding the CAR-engager protein or proteinaceous entities thereof in medium under conditions wherein the nucleic acid is expressed and isolating the CAR-engager protein or proteinaceous entities thereof from the cell and/or medium. In embodiments wherein the nucleic acid encodes proteinaceous entities, the proteinaceous entities are connected via click chemistry.

Another aspect of the present disclosure is directed to a method of treating cancer. The method entails administering to a subject an effective amount of a CAR-engager. In some embodiments, the subject will have received a prior administration of immune cells that express a CAR that contains an extracellular domain that binds an antigen present on a cancer cell that contains the ectodomain, and the ectodomain of the CAR-engager, a transmembrane domain, and an intracellular domain comprising a stimulatory domain.

Working examples disclosed herein demonstrate that the CAR-engager enhances CAR immune cells that target BCMA, drives them toward generation of memory cells, and prevents exhaustion of the CAR-immune cells.

### BRIEF DESCRIPTION OF THE DRAWINGS

FIG. 1 schematically illustrates the domains of a CAR-engager according to some embodiments that contains an ectodomain of an antigen on the surface of a cancer cell (Ag), a CH3 dimerization domain, and an immune cell effector domain (ICE). The CAR-engager may be a monomer, a dimer, or a multimer.

FIGS. 2A-2I are a set of illustrations and line plots that show three CAR-engagers. FIG. 2A schematically illustrates a CAR-engager that contains a BCMA ectodomain and a Neo2/15 synthetic cytokine immune cell effector domain. FIG. 2B schematically illustrates a CAR-engager that contains a BCMA ectodomain and two weak affinity mutated IL-2 (mIL2) synthetic cytokine immune cell effector domains. FIG. 2C schematically illustrates a CAR-engager that contains a BCMA ectodomain and a 4-1BBL immune cell effector domain. FIG. 2D is a line plot that shows dose-dependent staining of CAR T cells that bind CD19 or non-transduced T cells (NT T cells) with CAR-engager or control proteins. FIG. 2E is a line plot that shows dose-dependent staining of CAR T cells that bind BCMA or non-transduced T cells (NT T cells) with CAR-engager or control proteins. FIGS. 2F and 2G are a line and bar plot, respectively, that together show dose-dependent activation of CAR T cells after CAR-engager treatment. FIG. 2H is a line plot showing that the BCMA-muIL2 CAR-engager does not block the killing efficacy of the CAR T cells. FIG. 2I is a line plot that shows phosphorylation of signal transducer and activator of transcription (STAT5) in the BCMA CAR T cells.

FIGS. 3A-3B are a set of illustrations and line plots showing the effects of CAR-engagers on non-transduced T cells. FIG. 3A schematically illustrates the experimental design. FIG. 3B is a set of line plots that show T cell count and carboxyfluorescein succinimidyl ester (CFSE) staining of non-transduced, activated T cells treated with teceleukin, a CAR-engager containing an BCMA ectodomain and two mutated weak affinity IL-2 (muIL2), or CAR-engager containing an BCMA ectodomain and a Neoleukin domain.

FIGS. 4A-4C are a set of illustrations and line plots showing that CAR-engagers specifically activate CAR T cells. FIG. 4A schematically illustrates the experimental design. FIG. 4B is a bar plot that shows the percentage of CD69⁺ anti-BCMA CAR-transduced, activated T cells after treatment with BCMA CAR-engager, BCMA CAR-engager without an immune cell effector domain control, or a non-antigen-specific CAR-engager control. FIG. 4C is a bar plot that shows the percentage of CD69⁺ anti-CD19 CAR-transduced, activated T cells after treatment with CD19 CAR-engager or a non-antigen-specific CAR-engager control.

FIG. 5 is a line plot showing that CAR-engagers do not inhibit BCMA CAR T cell killing and that the percentage of OPM2 target cell survival after incubation with CAR T cells and CAR-engagers (red) or non-transduced T cells (blue).

FIGS. 6A-6C are a set of illustrations and photographs showing that CAR-engagers reduce tumor burden in vivo. FIG. 6A schematically illustrates the experimental design. FIGS. 6B-6C are a set of photographs that show tumor burden in mice before and after CAR T cell infusion and CAR-engager treatment.

FIGS. 7A-7C are a set of flow cytometry plots showing the tumor burden in mice after CAR T cell infusion and CAR-engager treatment. FIG. 7A is a set of flow cytometry plots that shows OPM2 tumor burden in blood, spleen, and lymph nodes. FIG. 7B is a set of flow cytometry plots that shows OPM2 tumor burden in bone marrow and lung. FIG. 7C is a set of flow cytometry plots that shows OPM2 tumor burden in liver, kidney, and the eye tumor site. eGFP (OPM2 cells) is shown on the y-axis and PerCP signal control is shown on the x-axis.

FIGS. 8A-8C are a set of flow cytometry plots showing human CD45⁻ and CAR− T cells in mice after CAR T cell infusion and CAR-engager treatment. FIG. 8A is a set of flow cytometry plots that shows CAR T cells in blood, spleen, and lymph nodes. FIG. 8B is a set of flow cytometry plots that shows CAR T cells in bone marrow and lung. FIG. 8C is a set of flow cytometry plots that shows CAR T cells in the liver, kidney, and the eye tumor site. CD45 staining is shown on the y-axis and CAR-engager labeled with AF647 staining is shown on the x-axis.

FIGS. 9A-9E are a set of schematics, line plots, and box plots showing that CAR-engager treatment results in enhanced activity and persistence of CAR T cells in vivo. FIG. 9A is a line plot that shows circulating half-life of the BCMA CAR-engagers. FIG. 9B schematically illustrates the experimental design. FIGS. 9C and 9D are a set of flow cytometry plots and box plots that show selective expansion and persistence of BCMA CAR T cells. FIG. 9E is a box plot that shows the percentage of CD8⁺ CAR T cells after CAR-engager treatment.

FIGS. 10A-10J are a set of schematics, survival, line, bar, and t-distributed stochastic neighbor embedding (tSNE) plots and photographs showing that CAR-engager treatment lowers the required dose of CAR T cells. FIG. 10A schematically illustrates the experimental design. FIG. 10B is a set of photographs that shows tumor burden in mice before and after CAR T cell infusion and CAR-engager treatment. FIG. 10C is a Kaplan-Meier plot that shows survival analysis. FIG. 10D is a line plot that shows flow cytometric analyses of CAR T cells in blood samples. FIG. 10E is a set of bar plots that show generation of memory CAR T cells. FIGS. 10F and 10G are a set of flow cytometric plots and bar plots showing that a substantial number of CAR T cells two months post-CAR T cell injection. FIG. 10H is a line plot showing that mice maintained consistent body weight throughout the experiment. FIG. 10I is a set of flow cytometric plots that show CAR T cells from CAR-engager treated mice have a stem-cell memory phenotype. FIG. 10J is a set of tSNE plots displaying FlowSOM defined clusters among persisting BCMA CAR T cells.

FIGS. 11A-11E are a set of schematics, photographs, and line, bar, and tSNE plots showing that CAR-engager treatment results in CAR T cell persistence in vivo. FIG. 11A schematically illustrates the experimental design. FIG. 11B is a set of photographs that show tumor burden in mice before and after CAR T cell infusion and CAR-engager treatment. FIG. 11C is a set of flow cytometric plots showing persistence of CAR T cells. FIG. 11D is a set of bar plots showing in vitro killing assays of persistent T cells. FIG. 11E is a set of t-SNE plots showing immune cell markers from CD8⁺ T cells.

FIGS. 12A-12B are a schematic and a set of bar plots showing that CAR-E treatment expands CAR T cells in vivo in the absence of tumor cells. FIG. 12A schematically illustrates the experimental design. FIG. 12B is a set of bar plots that show counts of CAR T cells 30 days-post injection.

FIGS. 13A-13B are a set of flow cytometry plots showing that neither the BCMA-muIL2 nor the VHH-muIL2 treatment exhibited binding to any specific population within human PBMCs. PBMCs were labeled with various markers to pre-gate B cells (CD20), T cells (CD3), or myeloid cells (CD11b). Cells were stained using different concentrations of the treatments followed by an anti-FLAG-Alexa647 secondary staining. FIG. 13A is a set of flow cytometry plots showing that BCMA-muIL2 does not bind to human PBMCs. FIG. 13B is a set of flow cytometry plots showing that VHH-muIL2 does not bind to human PBMCs.

FIGS. 14A-14C are a set of photomicrographs and dot plots showing specific binding and gradual internalization of the BCMA-muIL2 in CAR T cells. FIG. 14A is a set of photomicrographs that show cells stained with CellTracker Blue CMAC, incubated with the indicated treatment, each treatment labeled with Alexa647 (BCMA-muIL2) or dsRed (VHH-muIL2) for 1 to 5 hours and imaged. Photomicrographs are representative of >100 cell images. FIG. 14B is a dot plot that shows quantitative analysis of the imaged cells. FIG. 14C is a dot plot that shows the correlation between Alexa647 mean intensity (BCMA-muIL2) and dsRed mean intensity (VHH-muIL2).

FIGS. 15A-15C are a set of flow cytometry plots showing individual flow cytometric data corresponding to the pooled data presented in FIG. 9D. FIG. 15A is a set of flow cytometry results from mice treated solely with CAR T cells. FIG. 15B is a set of flow cytometry results from mice treated with CAR T cells and VHH-muIL2. FIG. 15C is a set of flow cytometry results from mice treated with CAR T cells and BCMA-muIL2.

FIGS. 16A-16C are a set of flow cytometry plots showing individual flow cytometric data of the mice shown in FIGS. 10A-10J. FIG. 16A is a set of flow cytometric results from mice treated solely with CAR T cells. FIG. 16B is a set of flow cytometry results from mice treated with CAR T cells and VHH-muIL2. FIG. 16C is a set of flow cytometry results from mice treated with CAR T cells and BCMA-muIL2.

FIGS. 17A-17C are a set of bar, line, and tSNE plots showing human T cell-derived cytokines in the serum of mice that received OPM2 cancer cells followed by a low dose of CAR T cells. FIG. 17A is a bar plot that shows levels of IFNγ, GM-CSF, and TNFα. Serum samples were diluted at a ratio of 1:40. The same plates were used to incubate both the standard samples and the serum samples, and a standard curve was plotted for each cytokine. FIG. 17B is a set of line plots that shows IFNγ levels between the BCMA-muIL2 group and the VHH-muIL2 group (error bars represent mean with standard deviation). FIG. 17C is a set of Flt-SNE mapping of CAR T cells derived from the PBS, BCMA-muIL2 and VHH-muIL2 treated mice showing the expression of ten immune cell markers.

FIG. 18 is a set of t-SNE mapping of CD4⁺ CAR⁺ T cells derived from the five BCMA-muIL2 CAR-E treated mice showing the expression of nine immune cell markers.

FIGS. 19A-19G are a set of flow cytometry, tSNE, bar, violin, and pie plots and heatmaps showing single-cell RNA sequencing analyses elucidate BCMA-muIL2 effect on CAR T cells. FIG. 19A is a set of flow cytometry plots showing CAR⁺ cells analyzed 89 days after CAR-T administration. FIG. 19B is a tSNE plot that shows data after Harmony algorithm, showing proportion of CD4, CD8, and proliferating (CD4 and CD8) cells. FIG. 19C is a tSNE plot that shows split between the groups treated with BCMA-muIL2 or VHH-muIL2 treatments. FIG. 19D is a set of heatmaps of significantly differentially expressed genes in CD4⁺ CAR T cells and CD8⁺ CAT T cells after the indicated treatment. FIG. 19E is a set of violin plots of the gene scores between CD8 and CD4 cells, the scores being constructed using the normalized expression of the different genes for each phenotype in FIG. 19D. FIG. 19F is set of pie plots that shows the diversity of T-cell receptor (TCR) clonotypes. FIG. 19G is a bar plot that shows clonotype diversity within a sample's total cell count.

FIGS. 20A-20B are a set of schematics and flow cytometry plots showing that CAR-engager treatment results in enhanced organ trafficking of CAR T cells in vivo. FIG. 20A schematically illustrates the experimental design. FIG. 20B is a set of flow cytometry plots that show selective trafficking, expansion, and persistence of BCMA CAR T cells.

FIGS. 21A-21C are a set of line and bar plots showing the effects of CAR-engagers on non-transduced T cells and CAR T cells. FIGS. 21A and 21B are a line and bar plot, respectively, that together show dose-dependent activation of CAR T cells after CAR-engager treatment (FIG. 21A) and that the CAR-engager does not activate non-transduced T cells (FIG. 21B). FIG. 21C is a line plot showing that the CD19-muIL2 CAR-engager does not block the killing efficacy of the CD19 CAR T cells or non-transduced T cells (NT T cells).

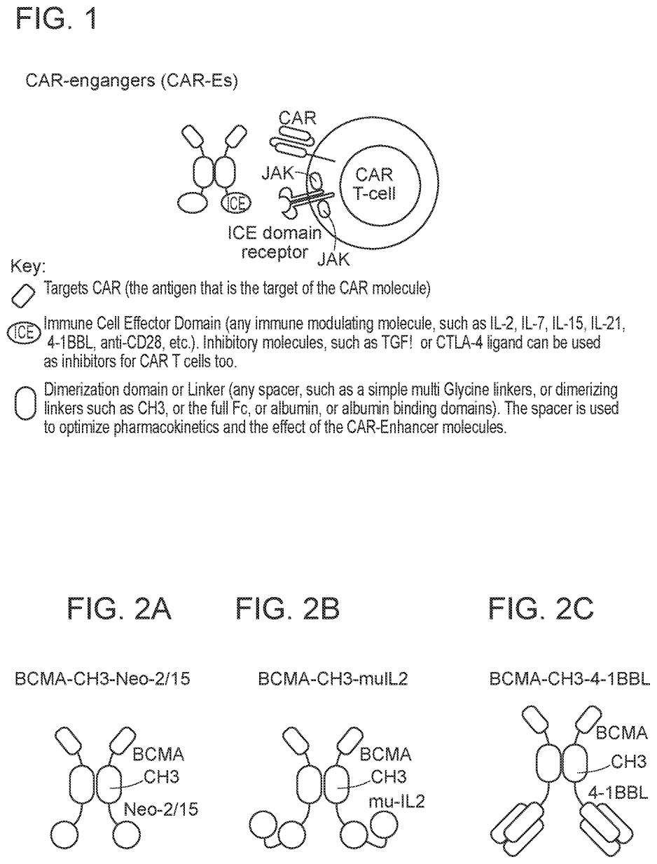

### DETAILED DESCRIPTION OF THE DISCLOSURE

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated to facilitate the understanding of the present disclosure.

As used in the description and the appended claims, the singular forms “a”, “an”, and “the” mean “one or more” and therefore include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “approximately” as used herein refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The transitional term “comprising,” which is synonymous with “including,” “containing,” or “characterized by,” is inclusive or open-ended and does not exclude additional, unrecited elements or method steps. By contrast, the transitional phrase “consisting of” excludes any element or method step not specified in the claim (or the specific element or method step with which the phrase “consisting of” is associated). The transitional phrase “consisting essentially of” limits the scope of a claim to the specified elements and method or steps and “unrecited elements and method steps that do not materially affect the basic and novel characteristic(s)” of the claimed disclosure.

### CAR-Engager

In one aspect, the disclosure provides a CAR-engager, also referred to herein as a CAR-enhancer, which contains a first proteinaceous entity and a second proteinaceous entity. The first proteinaceous entity comprises an ectodomain of an antigen present on a cancer cell (also referred to herein as a cancer antigen) and the second proteinaceous entity comprises an immune cell effector domain connected to the ectodomain. The term “antigen” as used herein refers to a target protein expressed by a cancer (e.g., tumor) cell. The ectodomain of an antigen is at least a portion of the antigen that is exposed on the cancer cell surface. The ectodomain binds an extracellular domain of the CAR presented on the immune cell, which is used with the CAR-engager used in the present methods. The immune cell effector domain binds to a cognate receptor on the same immune cell.

In some embodiments, CAR-engager is a contiguous protein, where the first proteinaceous entity and the proteinaceous entity are connected by a peptide bond. In some embodiments, the first proteinaceous entity and the second proteinaceous entity are connected by click chemistry.

In some embodiments, the CAR-engager is formulated and administered as a monomeric protein or proteinaceous entity. In other embodiments, the CAR-engager is formulated and administered in the form of a dimer, either as a homodimer or a heterodimer protein or proteinaceous entity.

### Ectodomain

The ectodomain of the CAR-engager binds an extracellular domain of a CAR presented on an immune cell. As is known in the art, the ectodomain of a cancer antigen is the portion of the antigen on the surface of a cancer cell that binds a T cell receptor or a CAR on an immune cell. The binding between CAR and cancer antigen may be direct or indirect. In direct binding embodiments, the ectodomain may be formed by contiguous or non-contiguous amino acid residues in the extracellular domain of a cancer antigen or may be an antibody or antibody fragment (including nanobody and nanobody fragments) that binds the CAR presented on an immune cell. In some embodiments, the CAR-engager may include the entire extracellular domain of a cancer antigen. Ectodomains may be derived from (e.g., identified in) a cancer antigen in accordance with standard techniques. See, e.g., and Gershoni et al., Biodrugs 21(3):145-156 (2007) and Francino-Urdaniz and Whitehead, RSC Chem. Biol. 2(6):1580-1589 (2021). The term “derived from” as used herein when referring to a protein and nucleic acid refers to a sequence that originates and is identified from the sequence of a parent (e.g., wild-type or endogenous) protein and nucleic acid, respectively. A sequence derived from a parent sequence may be identical, may be a portion of the parent sequence, or may have at least one variant from the parent sequence. Variants may include substitutions, insertions, or deletions. Thus, for example, an amino acid sequence derived from a parent sequence may be identical for a specific range of amino acids of the parent but does not include amino acids outside that specific region.

The amino acid sequences of representative cancer antigens from which an ectodomain may be derived are provided at the NCBI Accession numbers set forth in Table 1, and are incorporated herein by reference.

TABLE 1
Gene Name, Symbols, and NCBI Accession Numbers of Representative Cancer Antigens

| Gene Name | Gene Symbols | Protein Accession No(s). |
|---|---|---|
| Alpha fetoprotein | AFP, HPAFP, FETA, AFPD | NP_001125, NP_001341646 |
| Alkaline phosphatase, | ALP, ALPI, ALPP, IAP, | NP_001623 |
| placental | PALP, PLAP, PLAP-1 | |
| Axl receptor tyrosine | AXL, UFO, JTK11, Tyro7, | NP_001265528, NP_001690, |
| kinase | ARK | NP_068713 |
| Beta-1,4-n-acetyl- | B4GALNT1, GALGT, 48, | XP_005268830, XP_011536449, |
| galactosaminyltransferase 1 | GalNAc-T, SPG26, | XP_016874629, XP_016874631, |
| | GALNACT, SIAT2 | XP_024304696, XP_047284636, |
| | | XP_047284637, XP_047284638, |
| | | NP_001263397, NP_001263398, |
| | | NP_001469 |
| Tumor necrosis factor | TNFRSF17, BCM, BCMA, | NP_001183.2 |
| receptor superfamily | CD269 | |
| member 17 | | |
| Carbonic anhydrase 9 | CA9, CAIX, MN, P54/58N, | XP_047279805, XP_047279806, |
| | CA-IX, PMW1, G250 | NP_001207 |
| CD5 molecule | CD5, LEU1, T1 | NP_001333385, NP_055022 |
| CD7 molecule | CD7, GP40, TP41, LEU-9, | NP_006128 |
| | Tp40 | |
| CD19 molecule | CD19, CVID3, B4 | XP_011544283, NP_001171569, |
| | | NP_001372661, NP_001761 |
| CD20 molecule | See Membrane spanning 4- | |
| | domains A1 entry below | |
| CD22 molecule | CD22, SIGLEC2, SIGLEC-2, | NP_001172028, NP_001172029, |
| | BL-CAM, Siglec-2 | NP_001172030, NP_001265346, |
| | | NP_001762 |
| Fc epsilon receptor II | FCER2, CLEC4J, FCErII, | XP_005272519, NP_001193948, |
| | CD23A, CD23, FCE2, | NP_001207429, NP_001993 |
| | BLAST-2, IGEBF | |
| CD33 molecule | CD33, SIGLEC3, SIGLEC-3, | XP_011525833, XP_011525834, |
| | P67, FLJ00391, Gp67, Siglec- | XP_016882997, XP_016882998, |
| | 3 | XP_016882999, XP_047295684, |
| | | XP_047295685, XP_047295686, |
| | | XP_047295687, XP_047295688, |
| | | NP_001076087, NP_001171079, |
| | | NP_001763 |
| CD34 molecule | CD34 | NP_001020280, NP_001764 |
| CD38 molecule | CD38, ADPRC 1, CADPR1, | NP_001766 |
| | ADPRC1, T10 | |
| CD44 molecule | CD44, HUTCH-I, HCELL, | XP_005253288, XP_005253289, |
| | CSPG8, MC56, Pgp1, | XP_005253292, XP_005253295, |
| | MDU2, MDU3, MIC4, In | XP_005253296, XP_005253297, |
| | some embodiments,, Hermes- | XP_006718451, XP_006718453, |
| | 1, ECMR-III, HUTCH-1, | XP_011518784, XP_011518785, |
| | ECM-III, Epican, PGP-1, | XP_011518786, XP_011518787, |
| | CD44R, CDw44, H-CAM, | XP_011518788, XP_011518789, |
| | LHR, CDW44, PGP-I | XP_011518790, XP_011518791, |
| | | XP_016874072, XP_047283851, |
| | | XP_047283852, XP_047283853, |
| | | XP_047283854, XP_047283855, |
| | | XP_047283856, XP_047283857, |
| | | XP_047283858, XP_047283859, |
| | | XP_047283860, NP_000601, |
| | | NP_001001389, NP_001001390, |
| | | NP_001001391, NP_001001392, |
| | | NP_001189484, NP_001189485, |
| | | NP_001189486 |
| CD52 molecule | CD52, HE5, EDDM5, | NP_001794 |
| | CDW52, CDw52, He5 | |
| CD70 molecule | CD70, CD27L, CD27-L, | NP_001243, NP_001317261 |
| | CD27LG, TNFSF7, | |
| | TNLG8A, LPFS3 | |
| CD80 molecule | B7, B7-1, B7.1, BB1, | NP_005182 |
| | CD28LG, CD28LG1, CD80, | |
| | LAB7 | |
| CD86 molecule | B7-2, B7.2, B70, BU63, | NP_001193853, NP_001193854, |
| | CD28LG2, CD86, FUN-1, | NP_008820, NP_787058, |
| | LAB72 | NP_795711 |
| Basigin | 5F7, BSG, Basigin, CD147, | NP_001309172, NP_001719, |
| | EMMPRIN, EMPRIN, | NP_940991, NP_940992, |
| | HAb18G, OK, TCSF | NP_940993 |
| Interleukin 3 receptor | CD123, HIL-3Ra, IL-3RA, | XP_005274488, XP_005274489, |
| subunit alpha | IL3R, IL3RA, IL3RAY, | XP_005274837, XP_005274838, |
| | IL3RX, IL3RY | XP_016884980, XP_016885532, |
| | | XP_047298046, XP_047298047, |
| | | XP_047298686, XP_047298687, |
| | | NP_001254642, NP_002174 |
| Prominin 1 | AC133, CD133, CORD12, | XP_005248252, XP_005248253, |
| | HProminin, MCDR2, | XP_006714037, XP_011512195, |
| | MSTP061, PROM1, | XP_011512196, XP_011512197, |
| | PROML1, RP41, STGD4 | XP_011512199, XP_011512202, |
| | | XP_011512204, XP_011512205, |
| | | XP_016864289, XP_047272326, |
| | | XP_047272328, XP_047272329, |
| | | XP_047272330, XP_047272331, |
| | | XP_047272332, XP_047272333, |
| | | XP_047272334, XP_047272335, |
| | | NP_001139319, NP_001139320, |
| | | NP_001139321, NP_001139322, |
| | | NP_001139323, NP_001139324, |
| | | NP_001358335, NP_001358336, |
| | | NP_001358337, NP_006008 |
| Fucosyltransferase 3 | CD174, FT3B, FUT3, FucT- | NP_000140, NP_001091108, |
| (lewis blood group) | III, LE, Les, Lewis FT | NP_001091109, NP_001091110, |
| | | NP_001361669, NP_001369673, |
| | | NP_001369674, NP_001369675, |
| | | NP_001369676, NP_001369677, |
| | | NP_001369678, NP_001369679 |
| CD274 molecule | B7-H, B7-H1, B7H1, CD274, | XP_047279218, NP_001254635, |
| | HPD-L1, PD-L1, PDCD1L1, | NP_001300958, NP_054862 |
| | PDCD1LG1, PDL1 | |
| CD276 molecule | 4Ig-B7-H3, B7-H3, B7H3, | XP_005254757, XP_011520397, |
| | B7RP-2, CD276 | XP_016878127, XP_047289103, |
| | | XP_047289104, NP_001019907, |
| | | NP_001316557, NP_001316558, |
| | | NP_079516 |
| CD248 molecule | CD164L1, CD248, TEM1 | NP_065137 |
| carcinoembryonic | CD66e, CEA, CEACAM5 | XP_011524624, XP_016881634, |
| antigen (CEA) cell | | XP_016881635, NP_001278413, |
| adhesion molecule 5 | | NP_001295327, NP_004354 |
| C-type lectin domain | CD371, CLEC12A, CLL-1, | XP_005253381, XP_006719096, |
| family 12 member A | CLL1, DCAL-2, DCAL2, | XP_006719098, XP_006719099, |
| | MICL | XP_011518872, XP_011518873, |
| | | XP_011518875, XP_047284353, |
| | | XP_047284355, XP_047284356, |
| | | XP_047284357, XP_047284358, |
| | | NP_001193939, NP_001287659, |
| | | NP_612210, NP_963917 |
| Claudin 1 | CLD1, CLDN1, Claudin 1, | NP_066924 |
| | Claudin-1, ILVASC, SEMP1 | |
| Claudin 6 | CLDN6, Claudin 6, Claudin- | NP_067018 |
| | 6, Skullin | |
| Claudin 18 | CLDN18, SFTA5, SFTPJ | NP_057453 |
| Claudin 18.2 | CLDN18.2 | NP_001002026 |
| Cytotoxic T-lymphocyte | ALPS5, CD, CD152, | NP_001032720, NP_005205 |
| associated protein 4 | CELIAC3, CTLA-4, CTLA4, | |
| | GRD4, GSE, IDDM12 | |
| Cancer/testis antigen 1B | CT6.1, CTAG, CTAG1, | NP_001318 |
| | CTAGIB, ESO1, LAGE-2, | |
| | LAGE2, LAGE2A, LAGE2B, | |
| | NY-ESO-1 | |
| Chondroitin sulfate | CSPG4, CSPG4A, HMW- | XP_047288152, NP_001888 |
| proteoglycan 4 | MAA, MCSP, MCSPG, | |
| | MEL-CSPG, MSK16, NG2 | |
| Delta like canonical | DLL3, Delta3, SCDO1 | NP_058637, NP_982353 |
| notch ligand 3 | | |
| Epidermal growth factor | EGFR, EGFR VIII, ERBB, | XP_047275908, XP_047275909, |
| receptor | ERBB1, ERRP, HER1, | NP_001333826, NP_001333827, |
| | MENA, NISBD2, PIG61 | NP_001333828, NP_001333829, |
| | | NP_001333870, NP_005219, |
| | | NP_958439, NP_958440, |
| | | NP_958441 |
| Epithelial cell adhesion | Ber-Ep4, BerEp4, CD326, | NP_002345 |
| molecule | CO-17A, DIAR5, EGP, EGP- | |
| | 2, EGP314, EGP34, EGP40, | |
| | EPCAM, ESA, Ep-CAM, | |
| | GA733-2, HEA125, | |
| | HEGP314, HNPCC8, KS1/4, | |
| | KSA, Ly74, M1S2, M4S1, | |
| | MH99, MIC18, MK-1, MOC- | |
| | 31, MOC31, TACST-1, | |
| | TACSTD1, TROP1 | |
| EPH receptor A2 | ARCC2, CTPA, CTPP1, | XP_016856026, XP_047304215, |
| | CTRCT6, EC 2.7.10, ECK, | XP_047304223, XP_047304228, |
| | EPHA2, EphA2 | NP_001316019, NP_004422 |
| Erb-B2 receptor tyrosine | C-ERB-2, C-ERB2, CD340, | XP_047291546, NP_001005862, |
| kinase 2 | ERBB2, HER-2, HER-2/Neu, | NP_001276865, NP_001276866, |
| | HER2, Herstatin, MLN 19, | NP_001276867, NP_001369711, |
| | MLN-19, MLN19, NEU, | NP_001369712, NP_001369713, |
| | NGL, P185erbB2, TKR1, | NP_001369714, NP_001369715, |
| | VSCN2 | NP_001369716, NP_001369717, |
| | | NP_001369718, NP_001369719, |
| | | NP_001369720, NP_001369721, |
| | | NP_001369722, NP_001369723, |
| | | NP_001369724, NP_001369725, |
| | | NP_001369726, NP_001369727, |
| | | NP_001369728, NP_001369729, |
| | | NP_001369730, NP_001369731, |
| | | NP_001369732, NP_001369733, |
| | | NP_001369734, NP_001369735, |
| | | NP_004439 |
| Fibroblast activation | DPPIV, FAP, FAPA, FAPα, | XP_011509098, XP_011509099, |
| protein α | SIMP, Seprase | XP_016859074, NP_001278736, |
| | | NP_004451 |
| Folate hydrolase 1 | FGCP, FOLH, FOLH1, | XP_011518260, XP_016872921, |
| | GCP2, GCPII, MGCP, | XP_016872922, XP_016872923, |
| | NAALAD1, NAALAdase, | XP_047282634, XP_047282635, |
| | PSM, PSMA | XP_047282636, XP_047282637, |
| | | XP_047282638, NP_001014986, |
| | | NP_001180400, NP_001180401, |
| | | NP_001180402, NP_001338165, |
| | | NP_004467 |
| Folate receptor α | FBP, FOLR, FOLR1, FR-α, | NP_000793, NP_057936, |
| | FRα, FRα, NCFTD | NP_057937, NP_057941 |
| Glypican 3 | DGSX, GPC3, GTR2-2, | XP_016884902, NP_001158089, |
| | MXR7, OCI-5, OCI5, SDYS, | NP_001158090, NP_001158091, |
| | SGB, SGBS, SGBS1 | NP_004475 |
| G protein-coupled | GPRC5D | XP_016875072, NP_061124 |
| receptor class C group 5 | | |
| member D | | |
| Glycoprotein Nmb | GPNMB, HGFIN, NMB, | XP_005249635, XP_016867167, |
| | PLCA3 | XP_047275732, NP_001005340, |
| | | NP_002501 |
| Human papillomavirus | E7 | NP_041326 |
| transforming early | | |
| protein 7 | | |
| Interleukin 1 receptor | C3orf13, IL-1R-3, IL-1R3, | XP_016861837, XP_047304037, |
| accessory protein | IL-1RAcP, ILIR3, IL1RAP | XP_047304038, XP_047304039, |
| | | XP_047304040, XP_047304041, |
| | | XP_047304042, NP_001161400, |
| | | NP_001161401, NP_001161402, |
| | | NP_001161403, NP_001351808, |
| | | NP_001351809, NP_001351810, |
| | | NP_002173, NP_608273 |
| Interleukin 3 receptor | CD123, HIL-3Rα, IL-3RA, | XP_005274488, XP_005274489, |
| subunit α | IL3R, IL3RA, IL3RAY, | XP_005274837, XP_005274838, |
| | IL3RX, IL3RY | XP_016884980, XP_016885532, |
| | | XP_047298046, XP_047298047, |
| | | XP_047298686, XP_047298687, |
| | | NP_001254642, NP_002174 |
| Interleukin 6 receptor | CD126, Gp80, HIES5, IL- | XP_005245196, XP_016856688, |
| | 1Ra, IL-6R, IL-6R 1, IL-6R- | XP_047275604, XP_047275605, |
| | 1, IL-6RA, IL6Q, IL6QTL, | XP_047275606, XP_047275610, |
| | IL6R, IL6RA, IL6RQ | XP_047275611, XP_047275612, |
| | | XP_047275613, NP_000556, |
| | | NP_001193795, NP_001369698, |
| | | NP_001369699, NP_001369700, |
| | | NP_001369701, NP_001369702, |
| | | NP_001369703, NP_852004 |
| Interleukin 13 receptor | CD213A2, CD213α2, CT19, | NP_000631 |
| subunit α 2 | IL-13R, IL-13RA2, IL13BP, | |
| | IL13R, IL13RA2 | |
| Kinase insert domain | CD309, FLK-1, FLK1, KDR, | NP_002244 |
| receptor | VEGFR, VEGFR-2, VEGFR2 | |
| Kit proto-oncogene, | C-Kit, CD117, KIT, MASTC, | NP_000213, NP_001087241, |
| receptor tyrosine kinase | PBT, SCFR | NP_001372213, NP_001372214, |
| | | NP_001372215, NP_001372217, |
| | | NP_001372219, NP_001372221 |
| Killer cell lectin like | CD314, D12S2489E, KLR, | NP_031386 |
| receptor K1 | KLRK1, NKG2-D, NKG2D | |
| L1 cell adhesion | CAML1, CD171, HSAS, | NP_000416, NP_001137435, |
| molecule | HSAS1, LICAM, MASA, | NP_001265045, NP_076493 |
| | MIC5, N-CAM-LI, N- | |
| | CAML1, NCAM-L1, S10, | |
| | SPG1 | |
| Mage family member A1 | CT1.1, MAGE1, MAGE1A, | NP_004979 |
| | MAGEA1, MGC9326 | |
| Mage family member A4 | CT1.4, MAGE-41, MAGE- | NP_001011548, NP_001011549, |
| | X2, MAGE4, MAGE4A, | NP_001011550, NP_001373125, |
| | MAGE4B, MAGEA4, | NP_001373126, NP_001373127, |
| | MGC21336 | NP_001373128, NP_001373129, |
| | | NP_001373131, NP_001373132, |
| | | NP_002353 |
| Met proto-oncogene, | AUTS9, C-Met, DA11, | XP_011514525, XP_047276356, |
| receptor tyrosine kinase | DFNB97, HGFR, MET, | NP_000236, NP_001120972, |
| | RCCP2 | NP_001311330, NP_001311331 |
| Membrane | CALLA, CD10, CMT2T, | XP_006713710, XP_011511158, |
| metalloendopeptidase | EPN, MME, NEP, SCA43, | XP_011511159, XP_047304113, |
| | SFE | NP_000893, NP_001341571, |
| | | NP_001341572, NP_001341573, |
| | | NP_009218, NP_009219, |
| | | NP_009220 |
| Mesothelin | CAK1, MPF, MSLN, SMRP | NP_001170826, NP_005814, |
| | | NP_037536 |
| Mucin 1, cell surface | ADMCKD, ADMCKD1, | NP_001018016, NP_001018017, |
| associated | ADTKD2, CA 15-3, CD227, | NP_001037855, NP_001037856, |
| | Ca15-3, EMA, Episialin, | NP_001037857, NP_001037858, |
| | H23AG, KL-6, MAM6, | NP_001191214, NP_001191215, |
| | MCD, MCKD, MCKD1, | NP_001191216, NP_001191217, |
| | MUC-1, MUC-1/SEC, MUC- | NP_001191218, NP_001191219, |
| | 1/X, MUC1, MUC1/ZD, | NP_001191220, NP_001191221, |
| | Mucin-1, PEM, PEMT, PUM | NP_001191222, NP_001191223, |
| | | NP_001191224, NP_001191225, |
| | | NP_001191226, NP_001358649, |
| | | NP_002447 |
| Mucin 16, cell surface | CA-125, CA125, FLJ14303, | NP_001388430, NP_078966 |
| associated | MUC-16, MUC16, Mucin-16 | |
| Membrane spanning | B1, Bp35, CD20, CVID5, | NP_068769, NP_690605, |
| 4-domains A1 | FMC7, LEU-16, MS4A1, S7 | NP_690606 |
| Neural cell adhesion | CD56, MSK39, N-CAM-1, | NP_000606, NP_001070150, |
| molecule 1 | NCAM, NCAM-1, NCAM1 | NP_001229536, NP_001229537, |
| | | NP_001373218, NP_001373219, |
| | | NP_001373220, NP_001373221, |
| | | NP_001387532, NP_001387533, |
| | | NP_001387534, NP_001387535, |
| | | NP_001387536, NP_001387537, |
| | | NP_001387538, NP_001387539, |
| | | NP_001387540, NP_001387541, |
| | | NP_001387542, NP_001387543, |
| | | NP_001387544, NP_001387545, |
| | | NP_001387546, NP_001387547, |
| | | NP_001387548, NP_001387549, |
| | | NP_001387550, NP_001387551, |
| | | NP_001387552, NP_001387553, |
| | | NP_851996 |
| Programmed cell death 1 | CD279, HPD-1, HPD-L, | XP_006712636, NP_005009 |
| | HSLE1, PD-1, PD1, PDCD1, | |
| | SLEB2 | |
| Premelanosome protein | D12S53E, Gp100, HMB-45, | NP_001186982, NP_001186983, |
| | HMB45, ME20, ME20-M, | NP_001307050, NP_001307051, |
| | ME20M, P1, P100, PMEL, | NP_001371290, NP_008859 |
| | PMEL17, Pmel17, SI, SIL, | |
| | SILV | |
| Prominin 1 | AC133, CD133, CORD12, | XP_005248252, XP_005248253, |
| | HProminin, MCDR2, | XP_006714037, XP_011512195, |
| | MSTP061, PROM1, | XP_011512196, XP_011512197, |
| | PROML1, RP41, STGD4 | XP_011512199, XP_011512202, |
| | | XP_011512204, XP_011512205, |
| | | XP_016864289, XP_047272326, |
| | | XP_047272328, XP_047272329, |
| | | XP_047272330, XP_047272331, |
| | | XP_047272332, XP_047272333, |
| | | XP_047272334, XP_047272335, |
| | | NP_001139319, NP_001139320, |
| | | NP_001139321, NP_001139322, |
| | | NP_001139323, NP_001139324, |
| | | NP_001358335, NP_001358336, |
| | | NP_001358337, NP_006008 |
| Prostate stem cell antigen | LncPSCA, PRO232, PSCA | NP_005663 |
| Receptor tyrosine kinase | NTRKR1, ROR1 | XP_011539828, XP_016856865, |
| like orphan receptor 1 | | XP_016856866, NP_001077061, |
| | | NP_005003 |
| Receptor tyrosine kinase | BDB, BDB1, NTRKR2, | XP_005252065, XP_005252066, |
| like orphan receptor 2 | ROR2 | XP_006717184, XP_016870251, |
| | | XP_047279390, XP_047279391, |
| | | XP_047279392, XP_047279393, |
| | | NP_001305133, NP_004551 |
| Syndecan 1 | CD138, SDC, SDC1, SYND1 | XP_005262677, XP_005262678, |
| | | XP_005262679, NP_001006947, |
| | | NP_002988 |
| Slam family member 7 | 19A, CD319, CRACC, CS1, | XP_011508130, XP_011508131, |
| | Novel Ly9, SLAMF7 | XP_047282315, NP_001269517, |
| | | NP_001269518, NP_001269519, |
| | | NP_001269520, NP_001269521, |
| | | NP_001269522, NP_001269523, |
| | | NP_001269524, NP_001269525, |
| | | NP_067004 |
| TNF receptor | CD30, DIS166E, KI-1, Ki-1, | XP_011540743, XP_011540745, |
| superfamily member 8 | TNFRSF8 | XP_047290749, XP_047290755, |
| | | NP_001234, NP_001268359 |
| TNF receptor | CD262, DR5, KILLER, | NP_003833, NP_671716 |
| superfamily member 10B | TNFRSF10B, TRAIL-R2, | |
| | TRAILR2, TRICK2, | |
| | TRICK2A, TRICK2B, | |
| | TRICKB, ZTNFR9 | |
| TNF receptor | BAFF-R, BAFFR, BR3, | NP_443177 |
| superfamily member 13C | BROMIX, CD268, CVID4, | |
| | Prolixin, TNFRSF13C | |
| TNF receptor | BCM, BCMA, CD269, | NP_001183 |
| superfamily member 17 | TNFRSF13A, TNFRSF17 | |
| Tumor associated | EGP-1, EGP1, GA733-1, | NP_002344 |
| calcium signal transducer 2 | GA7331, GP50, M1S1, | |
| | TACSTD2, TROP2 | |
| Ubiquitin c-terminal | HEL-117, HEL-S-53, | NP_004172 |
| hydrolase L1 | NDGOA, PARK5, PGP 9.5, | |
| | PGP9.5, PGP95, SPG79, | |
| | UCH-L1, UCHL-1, UCHL1, | |
| | Uch-L1 | |
| UL16 binding protein 1 | N2DL-1, N2DL1, NKG2DL1, | XP_016866811, NP_001304018, |
| | RAETII, ULBP1 | NP_079494 |
| UL16 binding protein 2 | N2DL-2, N2DL2, NKG2DL2, | XP_016866810, XP_047275333, |
| | RAETIH, RAETIL, ULBP2 | NP_079493 |

The ectodomains are not limited to known cancer antigens. Unique cancer antigens (neoantigens) may be determined by known methods. For example, cancer genomes can be compared with normal cell genomes to identify neoantigens. In some embodiments, caner transcriptomes are compared to normal cell transcriptomes. Computational methods may then be utilized to identify suitable binding sites for a CAR. Most often the CAR binds a portion of the extracellular domain of an antigen. In some embodiments, the CAR and the corresponding cancer antigen are known in the art.

In some embodiments, the ectodomain of the CAR-engager contains the entire extracellular domain of a cancer antigen. In some embodiments, the CAR-engager contains a portion of the extracellular domain of a cancer antigen which is targeted by a CAR.

In some embodiments, the ectodomain of the CAR-engager contains the extracellular domain of BCMA. The amino acid sequence of a representative CAR-engager that contains a BCMA extracellular domain is

(SEQ ID NO: 1)
MLQMAGQCSQNEYFDSLLHACIPCQLRCSSNTPPLTCQRYCNASVTNSVK
GTNA.

In some embodiments, the ectodomain of the CAR-engager contains two repetitions of the extracellular domain of BCMA. The amino acid sequence of a representative CAR-engager that contains two repetitions of the BCMA extracellular domain is set forth below SE ID NO: 2):

| | |
|---|---|
| 1 | mlqmagqcsq neyfdsllha cipcqlrcss ntppltcqry |
| | cnasvtnsvk gtnagggsgg |
| 61 | gsprgsgggs mlqmagqcsq neyfdsllha cipcqlrcss |
| | ntppltcqry cnasvtnsvk |
| 121 | gtn |

In some embodiments, the ectodomain of the CAR-engager contains a variant of the extracellular domain of CD19. The amino acid sequence of a representative CAR-engager that contains a variant of CD19 extracellular domain is set forth below (SEQ ID NO: 3):

| | |
|---|---|
| 1 | peeplvvkve egdeawlpcl kgtsdgptqq ltwsresplk |
| | pflkvsfgvp glgvhvrpna |
| 61 | vslvisnvsq qmggfylcqp gppsekawqp gwtvnvegsg |
| | elfrwnvsdl gglgcglknr |
| 121 | ssegpsspsg klmspklyvw akdrpeiweg eppclpprds |
| | lnqslsrdmt vapgstlwls |
| 181 | cgvppdsvsr gplswthvhp kgpksilsle lkddrpardm |
| | wvtgtrlflp rataqdagky |
| 241 | ychrgnitms fhlevkarpy sahtklrtgg wk |

In some embodiments, the ectodomain has at least 85% sequence identity to SEQ ID NO: 3, at least 90% sequence identity to SEQ ID NO: 3, at least 95% sequence identity to SEQ ID NO: 3, at least 98% sequence identity to SEQ ID NO: 3, at least 99% sequence identity to SEQ ID NO: 3.

The amino acid sequence of a representative CAR-engager that contains a second variant of CD19 extracellular domain is set forth below (SEQ ID NO: 4):

| | |
|---|---|
| 1 | peeplvvkve egdeawlpcl kgtsdgptqq ltwsresplk |
| | pflkvsfgvp glgvhvrpna |
| 61 | vslvisqvsq qmggfylcqp gppsekawqp gwtvnvegsg |
| | elfrwgvsdl gglgcglkqr |
| 121 | ssegpsspsy klmspklyvw akdrpeiweg eppclpprds |
| | lqqslsrdmt vapgstlwls |
| 181 | cgvppdsvsr gplswthvhp kgpksllsle lkddrpardm |
| | wvtgtrlflp rataqdagky |
| 241 | ychrgqltms fhlevkarpv sahtklrtgg wk |

In some embodiments, the ectodomain of the CAR-engager contains the extracellular domain of CD19. The amino acid sequence of a representative CAR-engager that contains a CD19 extracellular domain set forth below (SEQ ID NO: 5):

| | |
|---|---|
| 1 | peeplvvkve egdnavlqcl kgtsdgptqq ltwsresplk |
| | pflklslglp glgihmrpla |
| 61 | iwlfifnvsq qmggfylcqp gppsekawqp gwtvnvegsg |
| | elfrwnvsdl gglgcglknr |
| 121 | ssegpsspsg klmspklyvw akdrpeiweg eppclpprds |
| | Inqslsqdlt mapgstlwls |
| 181 | cgvppdsvsr gplswthvhp kgpksllsle lkddrpardm |
| | wvmetglllp rataqdagky |
| 241 | ychrgnltms fhleitarpv lwhwllrtgg wk |

In some embodiments, the ectodomain of the CAR-engager contains a portion extracellular domain of CD19. In some embodiments, the ectodomain of the CAR-engager is KDRPEIWEGEPP (SEQ ID NO: 103), which corresponds to positions 142-153 of SEQ ID NO: 5.

In some embodiments, the ectodomain of the CAR-engager contains the extracellular domain of CD20. The amino acid sequence of a representative CAR-engager that contains a CD20 extracellular domain is KISHFLKMESLNFIRAHTPYINIYNCEPANPSEKNSPSTQYCYSIQS (SEQ ID NO: 6).

In some embodiments, the ectodomain of the CAR-engager contains the extracellular domain of CD22. The amino acid sequence of a representative CAR-engager that contains a CD22 extracellular domain is set forth below (SEQ ID NO: 7):

| | |
|---|---|
| 1 | dsskwvfehp etlyawegac vwipctyral dgdlesfilf |
| | hnpeynknts kfdgtrlyes |
| 61 | tkdgkvpseq krvqflgdkn knctlsihpv hlndsgqlgl |
| | rmesktekwm erihlnvser |
| 121 | pfpphiqlpp eiqesqevtl tcllnfscyg ypiqlqwlle |
| | gvpmrqaavt stsltiksvf |
| 181 | trselkfspq wshhgkivtc qlqdadgkfl sndtvqlnvk |
| | htpkleikvt psdaivregd |
| 241 | svtmtcevss snpeyttvsw lkdgtslkkq ntftlnlrev |
| | tkdqsgkycc qvsndvgpgr |
| 301 | seevflqvqy apepstvqil hspavegsqv eflcmslanp |
| | lptnytwyhn gkemqgrtee |
| 361 | kvhipkilpw hagtyscvae nilgtgqrgp gaeldvqypp |
| | kkvttviqnp mpiregdtvt |
| 421 | lscnynssnp svtryewkph gaweepslgv lkiqnvgwdn |
| | ttiacaacns wcswaspval |
| 481 | nvqyaprdvr vrkikplsei hsgnsvslqc dfssshpkev |
| | qffwekngrl lgkesqlnfd |
| 541 | sispedagsy scwvnnsigq taskawtlev lyaprilrvs |
| | mspgdqvmeg ksatltcesd |
| 601 | anppvshytw fdwnnqslpy hsqklrlepv kvqhsgaywc |
| | qgtnsvgkgr splstltvyy |
| 661 | spetigrr |

In some embodiments, the ectodomain of the CAR-engager contains a portion of the extracellular domain of CD22 (SEQ ID NO: 7). In some embodiments, the ectodomain of the CAR-engager contains the Ig domains 2-3 of CD22. The amino acid sequence of a representative CAR-engager that contains Ig domains 2-3 of CD22 is set forth below (SEQ ID NO: 104):

| | |
|---|---|
| 1 | phiqlppeiq esqevtltcl lnfscygypi qlqwllegvp |
| | mrqaavtsts ltiksvftrs |
| 61 | elkfspqwsh hgkivtcqlq dadgkflsnd tvqpkleikv |
| | tpsdaivreg dsvtmtcevs |
| 121 | ssnpeyttvs wlkdgtslkk qntftlnlre vtkdqsgkyc |
| | cqvsndvgpg rseevflq |

In some embodiments, the ectodomain of the CAR-engager contains the Ig domain 3 of CD22. The amino acid sequence of a representative CAR-engager that contains an Ig domain 3 of CD22 is set forth below (SEQ ID NO: 105):

| | |
|---|---|
| 1 | pkleikvtps daivregdsv tmtcevsssn peyttvswlk dgtslkkqnt ftlnlrevtk |
| 61 | dqsgkyccqv sndvgpgrse evflq |

In some embodiments, the ectodomain of the CAR-engager contains the Ig domains 5-7 of CD22. The amino acid sequence of a representative CAR-engager that contains Ig domains 5-7 of CD22 is set forth below (SEQ ID NO: 106):

| | |
|---|---|
| 1 | pkkvttviqn pmpiregdtv tlscnynssn psvtryewkp hgaweepslg vlkiqnvgwd |
| 61 | nttiacaacn swcswaspva lnprdvrvrk ikplseihsg nsvslqcdfs sshpkevqff |
| 121 | wekngrllgk esqlnfdsis pedagsyscw vnnsigqtas prrlrvsmsp gdqvmegksa |
| 181 | tltcesdanp pvshytwfdw nnqslpyhsq klrlepvkvq hsgaywcqgt nsvgkgrspl |
| 241 | stlt |

In some embodiments, the ectodomain of the CAR-engager contains the Ig domains 5-7 of CD22. The amino acid sequence of a representative CAR-engager that contains Ig domains 6-7 of CD22 is set forth below (SEQ ID NO: 107):

| | |
|---|---|
| 1 | aprdvrvrki kplseihsgn svslqcdfss shpkevqffw ekngrllgke sqlnfdsisp |
| 61 | edagsyscwv nnsigqtask awtlevlyap rrlrvsmspg dqvmegksat ltcesdanpp |
| 121 | vshytwfdwn nqslpyhsqk lrlepvkvqh sgaywcqgtn svgkgrspls tltvyyspet |
| 181 | igrr |

In some embodiments, the ectodomain of the CAR-engager contains an extracellular domain of Claudin 18.2. The amino acid sequence of a representative CAR-engager that contains a Claudin 18.2 first extracellular domain is set forth below (SEQ ID NO: 8):

| | |
|---|---|
| 1 | dqwstqdlyn npvtavfnyq glwrscvres sgftecrgyf tllglpamlq avr |

The amino acid sequence of a representative CAR-engager that contains a Claudin 18.2 second extracellular domain is set forth below (SEQ ID NO: 9):

| | |
|---|---|
| 1 | vtnfwmstan mytgmggmvq tvqtrytfga a |

In some embodiments, the ectodomain of the CAR-engager contains the extracellular domain of SLAMF7. The amino acid sequence of a representative CAR-engager that contains a SLAMF7 extracellular domain is set forth below SE ID NO: 10):

| | |
|---|---|
| 1 | sgpvkelvgs vggavtfplk skvkqvdsiv wtfnttplvt iqpeggtiiv tqnrnrervd |
| 61 | fpdggyslkl sklkkndsgi yyvgiysssl qqpstqeyvl hvyehlskpk vtmglqsnkn |
| 121 | gtcvtnltcc mehgeedviy twkalgqaan eshngsilpi swrwgesdmt ficvarnpvs |
| 181 | rnfsspilar klcedaaddp dssm |

In some embodiments, the ectodomain of the CAR-engager contains the extracellular domain of PD-1. The amino acid sequence of a representative CAR-engager that contains a PD-1 extracellular domain is set forth below (SEQ ID NO: 11):

| | |
|---|---|
| 1 | fldspdrpwn pptfspallv vtegdnatft csfsntsesf vlnwyrmsps nqtdklaafp |
| 61 | edrsqpgqdc rfrvtqlpng rdfhmsvvra rrndsgtylc gaislapkaq ikeslraelr |
| 121 | vterraevpt ahpspsprpa gqfqtlv |

In some embodiments, the ectodomain of the CAR-engager contains a variant of the extracellular domain of PD-1. In some embodiments, the ectodomain of the CAR-engager contains the N-loop of PD-1. The amino acid sequence of a representative CAR-engager that contains the N-loop of the PD-1 extracellular domain is LDSPDRPWNP (SEQ ID NO: 108), which corresponds to positions 2-11 of SEQ ID NO: 11.

In some embodiments, the ectodomain of the CAR-engager contains the CD-loop of PD-1. The amino acid sequence of a representative CAR-engager that contains the CD-loop of the PD-1 extracellular domain is NQTDKLAAFPEDRSQPGQDCRFRVTQ (SEQ ID NO: 109), which corresponds to positions 51-76 of SEQ ID NO: 11.

In some embodiments, the ectodomain of the CAR-engager contains the extracellular domain of KIT. The amino acid sequence of a representative CAR-engager that contains a KIT extracellular domain is set forth below (SEQ ID NO: 12):

| | |
|---|---|
| 1 | qpsvspgeps ppsihpgksd livrvgdeir llctdpgfvk wtfeildetn enkqnewite |
| 61 | kaeatntgky tctnkhglsn siyvfvrdpa klflvdrsly gkedndtlvr cpltdpevtn |
| 121 | yslkgcqgkp lpkdlrfipd pkagimiksv krayhrlclh csvdqegksv lsekfilkvr |
| 181 | pafkavpvvs vskasyllre geeftvtcti kdvsssvyst wkrensqtkl qekynswhhg |
| 241 | dfnyergatl tissarvnds gvfmcyannt fgsanvtttl evvdkgfini fpminttvfv |
| 301 | ndgenvdliv eyeafpkpeh qqwiymnrtf tdkwedypks enesniryvs elhltrlkgt |
| 361 | eggtytflvs nsdvnaaiaf nvyvntkpei ltydrlvngm lgcvaagfpe ptidwyfcpg |
| 421 | teqrcsasvl pydyqtlnss gppfgklvvq ssidssafkh ngtveckayn dvgktsayfn |
| 481 | fafkgnnkeq ihphtlftp |

In some embodiments, the ectodomain of the CAR-engager contains the extracellular domain of TROP2. The amino acid sequence of a representative CAR-engager that contains a TROP2 extracellular domain is set forth below SEQ ID NO: 13):

| | |
|---|---|
| 1 | htaaqdnctc ptnkmtvcsp dgpggrcqcr algsgmavdc stltskclll karmsapkna |
| 61 | rtlvrpseha lvdndglydp dcdpegrfka rqcnqtsvcw cvnsvgvrrt dkgdlslrcd |
| 121 | elvrthhili dlrhrptaga fnhsdldael rrlfreryrl hpkfvaavhy eqptiqielr |
| 181 | qntsqkaagd vdigdaayyf erdikgeslf qgrggldlrv rgeplqvert liyyldeipp |
| 241 | kfsmkrlt |

In some embodiments, the ectodomain of the CAR-engager contains the extracellular domain of CD38. The amino acid sequence of a representative CAR-engager that contains a CD38 extracellular domain is set forth below (SEQ ID NO: 14):

| | |
|---|---|
| 1 | vprwrqqwsg pgttkrfpet vlarcvkyte ihpemrhvdc qsvwdafkga fiskhpcnit |
| 61 | eedyqplmkl gtqtvpenki llwsrikdla hqftqvqrdm ftledtllgy laddltwcge |
| 121 | fntskinyqs cpdwrkdcsn npvsvfwktv srrfaeaacd vvhvmlngsr skifdknstf |
| 181 | gsvevhnlqp ekvqtleawv ihggredsrd lcqdptikel esiiskrniq fsckniyrpd |
| 241 | kflqcvknpe dssctsei |

In some embodiments, the ectodomain of the CAR-engager is derived from mesothelin (MSLN). MSLN is a GPI-anchored protein, therefore the entire MSLN protein is extracellular. The amino acid sequence of a representative MSLN is set forth below (SEQ ID NO: 15):

| | |
|---|---|
| 1 | malptarpil gscgtpalgs llfllfslgw vqpsrtlage tgqeaapldg vlanppniss |
| 61 | lsprqllgfp caevsglste rvrelavala qknvklsteq lrclahrlse ppedldalpl |
| 121 | dlllflnpda fsgpqactrf fsritkanvd llprgaperq rllpaalacw gvrgsllsea |
| 181 | dvralgglac dlpgrfvaes aevllprlvs cpgpldqdqq eaaraalqgg gppygppstw |
| 241 | svstmdalrg llpvlgqpii rsipqgivaa wrqrssrdps wrqpertilr prfrrevekt |
| 301 | acpsgkkare ideslifykk weleacvdaa llatqmdrvn aipftyeqld vikhkldely |
| 361 | pqgypesviq hlgylflkms pedirkwnvt sletlkalle vnkghemspq aprrplpqva |
| 421 | tlidrfvkgr gqldkdtldt ltafypgylc slspeelssv ppssiwavrp qdldtcdprq |
| 481 | ldvlypkarl afqnmngsey fvkiqsflgg aptedlkals qqnvsmdlat fmklrtdavl |
| 541 | pltvaevqkl lgphveglka eerhrpvrdw ilrqrqddld tlglglqggi pngylvldls |
| 601 | mqealsgtpc llgpgpvltv lalllastla |

In some embodiments, the ectodomain of the CAR-engager contains a portion of an extracellular domain of a cancer antigen. In some embodiments, the ectodomain of the CAR-engager contains a portion of the MSLN protein. In some embodiments, the ectodomain of the CAR-engager is IPNGYLVLDLSMQEALS (SEQ ID NO: 16). In some embodiments, the ectodomain of the CAR-engager is YNVNDLSMQEL (SEQ ID NO: 17), where N is any amino acid.

In some embodiments, the CAR-engager ectodomain is an antibody or antibody fragment that binds the CAR presented on an immune cell. In these embodiments, the CAR-engager ectodomain may be an antibody fragment, including a nanobody fragment. In some embodiments, the CAR-engager ectodomain is an antibody, or fragment thereof, directed to the CAR extracellular domain. In some embodiments, the CAR-engager ectodomain is an anti-mouse antibody that binds antibody fragments that originate from mouse sources, which has been incorporated into the CAR extracellular domain. See, Kochenderfer et al., J. Immunother. 32(7):689-702 (2009) and Cheng et al., Cytometry A. 103(1):16-26 (2023). In some embodiments, the CAR-engager ectodomain is an anti-idiotype antibody fragment, which binds the variable region of a CAR antibody fragment. In some embodiments, the CAR-engager ectodomain is an antibody, or fragment thereof, described in U.S. Pat. No. 9,701,758 and U.S. Patent Application Publication 2005/0287148, both of which are incorporated by reference in their entirety herein. In some embodiments, the CAR-engager ectodomain is the Peptostreptococcus magnus protein L (NCBI Accession number Q51918) which binds antibody and scFv VL regions of κ light chains.

### Indirect Binding Embodiments

Embodiments which rely on indirect binding between CAR and cancer antigen contain ectodomains comprising a cancer irrelevant antigen. The cancer irrelevant antigen is not an antigen on the surface of a cancer cell, but that of an exogenous antigen contained on a protein therapeutic. The protein therapeutic typically contains at least the cancer irrelevant antigen and a cancer antigen binding domain (e.g., an antibody or antibody fragment) and serves to redirect a CAR immune cell from the cancer irrelevant antigen to a cancer antigen. CARs that bind cancer irrelevant antigens are referred to as universal CARs or binary activated CARs (BAT-CARs). In some embodiments, the cancer irrelevant antigen is a fluorescent molecule (e.g., fluorescein, fluorescein isothiocyanate (FITC), an anthracene, an alexa fluor, a rhodamine, a rhodol, an acridine or a xanthene), 4-[(6-methylpyrazin-2-yl) oxy]benzoate (MPOB), anthraquinone-2-carboxylate (AQ), anthraquinone-2-acid, tetraxetan (DOTA), an amphetamine, a benzodiazepine, a benzoylecgonine, a buprenorphine, an opioid, a cannabinoid, a phencyclidine, a tricyclic antidepressant, dextromethorphan, fentanyl, meprobamate, methadone, methamphetamine, oxycodone, THC, tramadol, Zolpidem, ketamine, LSD, MDMA, methaqualone, propoxyphene, norketimine, biotin, or a leucine zipper. Additional cancer irrelevant antigens are described in, for example, U.S. Pat. No. 11,225,520, U.S. Patent Application Publications 2020/0306376, 2021/0137987, and 2021/0228699, and International Patent Application Publication WO 2023/060126, each of which is incorporated herein by reference.

### Immune Cell Effector Domain

The immune cell effector domain of the CAR-engager binds a cognate receptor on the immune cell that expresses the CAR-encoding nucleic acid. This binding event modulates the activity of the CAR-immune cell. The terms “modulate(s),” and “modulation” as used herein embrace both activation and inhibition of the CAR immune cell. Accordingly, the immune cell effector domain may be a cytokine, an immune cell-activating moiety, or an immune cell-inhibiting moiety, and variants and fragments thereof that bind to their cognate targets. The term “cytokine”, as is known in the art, includes low molecular weight extracellular polypeptides/glycoproteins that promote, modulate, and regulate the immune response (i.e., increase or decrease activity, differentiation, or proliferation). Representative examples of cytokines include chemokines, interferons (IFNs), interleukins (ILs), lymphokines and tumor necrosis factors (TNFs). The term “immune cell-activating variant” of a cytokine as used herein refers to non-naturally occurring variant of a cytokine capable of binding to a cytokine receptor on an immune cell and initiating signal transduction through that receptor to achieve substantially the same effect as the naturally occurring cytokine.

In some embodiments, the CAR-engager contains a plurality (i.e., two or more) of immune cell effector domains, any two or more of which may be the same as or different from each other. In some embodiments, the CAR-engager contains two immune cell effector domains. In some embodiments, the CAR-engager contains three immune cell effector domains.

In some embodiments, the immune cell effector domain is an immune cell activating moiety, e.g., immune cell activating cytokines and immune cell-activating variants and fragments thereof. Immune cell activating moieties activate, promote, or maintain the activity of immune cells.

In some embodiments, the immune cell effector domain is derived from CD40, CD48, CD58, CD70, CD80, CD86, CD112, glucocorticoid-induced TNFR-related protein ligand (GITRL; TNFSF18), herpesvirus entry mediator (HVEM; TNFSF14), Semaphorin 3B (SEMAA; SEMA3B), Signaling lymphocytic activation molecule family member 1 (SLAM; SLAMFI; CD150), T cell immunoglobulin and mucin domain containing 4 (TIM4), TNF superfamily member 4 (TNFSF4; OX40L), TNF superfamily member 8 (TNFSF8; CD30L), interleukin-2 (IL-2), IL-7, IL-9, IL-10, IL-12, IL-15, IL-18, IL-21, IL-27, CCL21, 4-1BBL (also known as TNF superfamily member 9; TNFSF9), or an immune cell-activating variant thereof.

The amino acid sequences of representative immune cell activating moieties (e.g., cytokines) are from which the immune cell effector domain may be derived are provided at the NCBI Accession numbers set forth in Table 2, and are incorporated herein by reference.

TABLE 2
Gene Name, Symbols, and NCBI Accession Numbers of Representative
Immune Cell-Activating Proteins

| Gene Name | Gene Symbols | Protein Accession No(s). |
|---|---|---|
| C—C motif chemokine | 6Ckine, CCL21, CKb9, ECL, | NP_002980 |
| ligand 21 | Exodus-2, SCYA21, SLC, | |
| | TCA4 | |
| CD40 molecule | Bp50, CD40, CDW40, | XP_005260676, XP_011527411, |
| | CDw40, P50, TNFRSF5 | XP_016883624, XP_016883625, |
| | | XP_047296557, NP_001241, |
| | | NP_001289682, NP_001309350, |
| | | NP_001309351, NP_001349687, |
| | | NP_690593 |
| CD48 molecule | BCM1, BLAST, BLAST1, | XP_005245682, XP_016858356, |
| | CD48, HCD48, MCD48, | XP_047290967, NP_001242959, |
| | MEM-102, SLAMF2, TCT.1 | NP_001769 |
| CD58 molecule | Ag3, CD58, LFA-3, LFA3 | NP_001138294, NP_001770 |
| CD70 molecule | CD27-L, CD27L, CD27LG, | NP_001243, NP_001317261 |
| | CD70, LPFS3, TNFSF7, | |
| | TNLG8A | |
| CD80 molecule | B7, B7-1, B7.1, BB1, | NP_005182 |
| | CD28LG, CD28LG1, CD80, | |
| | LAB7 | |
| CD86 molecule | B7-2, B7.2, B70, BU63, | NP_001193853, NP_001193854, |
| | CD28LG2, CD86, FUN-1, | NP_008820, NP_787058, |
| | LAB72 | NP_795711 |
| Nectin cell adhesion | CD112, HVEB, HveB, | XP_047295125, NP_001036189, |
| molecule 2 | NECTIN2, Nectin-2, PRR2, | NP_002847 |
| | PVRL2, PVRR2 | |
| Semaphorin 3b | 3B, LUCA-1, SEMA3B, | NP_001005914, NP_001276989, |
| | SEMA5, SEMAA, SemA, | NP_001276990, NP_001276991, |
| | Sema A(V), Sema V, Sema5, | NP_001276992, NP_004627 |
| | SemaV | |
| Signaling lymphocytic | CD150, CDw150, IPO-3, | XP_005245513, XP_016857619, |
| activation molecule | SLAM, SLAMF1 | XP_016857620, XP_047284442, |
| family member 1 | | XP_047284443, XP_047284446, |
| | | NP_001317683, NP_003028 |
| T cell immunoglobulin | SMUCKLER, TIM-4, TIM4, | XP_011532996, XP_016865510, |
| and mucin domain | TIMD-4, TIMD4 | NP_001140198, NP_612388 |
| containing 4 | | |
| TNF superfamily | CD134L, CD252, GP34, | XP_011508266, XP_016857717, |
| member 4 | Gp34, OX-40L, OX40L, | XP_047285852, XP_047285858, |
| | OX4OL, TNFSF4, TNLG2B, | XP_047285864, NP_001284491, |
| | TXGP1 | NP_003317 |
| TNF superfamily | CD153, CD30-L, CD30L, | NP_001235, NP_001239219 |
| member 8 | CD30LG, TNFSF8, TNLG3A | |
| TNF superfamily | CD258, HVEM-L, HVEML, | NP_001363816, NP_003798, |
| member 14 | LIGHT, LTg, TNFSF14 | NP_742011 |
| TNF superfamily | AITRL, GITRL, HGITRL, | NP_005083 |
| member 18 | TL6, TNFSF18, TNLG2A | |
| Interleukin 2 | IL-2, IL2, TCGF | NP_000577 |
| Interleukin 7 | IL-7, IL7 | XP_011515824, XP_011515825, |
| | | XP_047277721, XP_047277722, |
| | | XP_047277723, XP_047277724, |
| | | XP_054216412, XP_054216413, |
| | | XP_054216414, NP_000871, |
| | | NP_001186815, NP_001186816, |
| | | NP_001186817 |
| Interleukin 12a | CLMF, CLMF P35, IL-12A, | NP_000873, NP_001341511, |
| | IL12, IL12A, NFSK, NKSF1, | NP_001341512, NP_001384921 |
| | P35 | |
| Interleukin 12b | CLMF, CLMF P40, CLMF2, | NP_002178 |
| | IL-12B, IL12, IL12B, IMD28, | |
| | IMD29, NKSF, NKSF2 | |
| Interleukin 15 | IL-15, IL15, MGC9721 | NP_000576, NP_751915 |
| Interleukin 18 | IGIF, IL-18, IL-1g, IL18, | XP_011541107, NP_001230140, |
| | IL1F4 | NP_001373349, NP_001553 |
| Interleukin 21 | CVID11, IL-21, IL21, Za`1 | NP_001193935, NP_068575 |
| Interleukin 27 | IL-27, IL-27-A, IL-27A, | NP_663634 |
| | IL27, IL27-A, IL27A, | |
| | IL27p28, IL30, MGC71873, | |
| | P28 | |
| TNF superfamily | 4-1BB-L, 4-1BBL, CD137L, | NP_003802 |
| member 9 | TNFSF9, TNLG5A | |

In some embodiments, the immune cell effector domain is the wild-type IL-2, having the amino acid sequence set forth below (SEQ ID NO: 102; NCBI Accession No. NP 000577):

| | |
|---|---|
| 1 | aptssstkkt qlqlehllld lqmilnginn yknpkltrml tfkfympkka telkhlqcle |
| 61 | eelkpleevl nlaqsknfhl rprdlisnin vivlelkgse ttfmceyade tativeflnr |
| 121 | witfcqsiis tlt |

In some embodiments, the immune cell effector domain is a synthetic, i.e., non-naturally occurring IL-2, which is a variant of the wild-type IL-2 (SEQ ID NO: 102) in that it has an amino acid substitution at positions 16 and/or 42.

In some embodiments, the immune cell effector domain has an H16A substitution (i.e., an alanine (A) at position 16 in place of the histidine (H)) relative to SEQ ID NO: 102 and/or an F42A substitution (i.e., an alanine (A) at position 42 in place of the phenylalanine (F)) relative to SEQ ID NO: 102, both substituted alanine residues shown as boxed amino acids in SEQ ID NO: 19). In some embodiments, the immune cell effector domain is the weak affinity variant of IL-2 (muIL2), having the amino acid sequence set forth below (SEQ ID NO: 19), which contains the H16A and F42A substitutions, as follows:

| | |
|---|---|
| 1 | aptssstkkt qlqle [symbol] llld lqmilnginn yknpkltrml t [symbol] kfympkka telkhlqcle |
| 61 | eelkpleevl nlaqsknfhl rprdlisnin vivlelkgse ttfmceyade tativeflnr |
| 121 | witfcqsiis tlt |

By “weak affinity,” it is mean a the natural (wild-type) sequence of human IL-2 SEQ ID NO: 102) has higher affinity for the IL-2 receptor (IL-2R) than SEQ ID NO: 19.

More specifically, the muIL2 (SEQ ID NO: 19) immune cell effector domain has a dissociation constant (K_{D}) of about 1200 nM for IL-2Rα (CD25), representing a 110-fold decrease as compared to wild type IL-2, and a K_{D} of about 610 nM for IL-2Rβ, representing a 3-fold decrease as compared to wild type IL-2.

In some embodiments, the CAR-engager contains more than one immune cell effector domain. The immune cell effector domains in the CAR-engager can be the same or different.

In some embodiments, the CAR-engager contains two immune cell effector domains, e.g., first and the second immune cell effector domains that are the weak affinity IL-2 variants, having together, the amino acid sequence set forth below (SEQ ID NO: 20):

| | |
|---|---|
| 1 | aptssstkkt qlqleallld lqmilnginn yknpkltrml takfympkka telkhlqcle |
| 61 | eelkpleevl nlaqsknfhl rprdlisnin vivlelkgse ttfmceyade tativeflnr |
| 121 | witfcqsiis tltggggsgg ggsggggsgg ggsaptssst kktqlqleal lldlqmilng |
| 181 | innyknpklt rmltakfymp kkatelkhlq cleeelkple evlnlaqskn fhlrprdlis |
| 241 | ninvivlelk gsettimcey adetativef lnrwitfcqs iistlt |

In some embodiments, the immune cell effector domain may be derived from IL-7. The amino acid sequence of a representative IL-7 is set forth below (SEQ ID NO: 21):

| | |
|---|---|
| 1 | mfhvsfryif glpplilvll pvassdcdie gkdgkqyesv lmvsidqlld smkeigsncl |
| 61 | nnefnffkrh icdankegmf lfraarklrq flkmnstgdf dlhllkvseg ttillnctgq |
| 121 | vkgrkpaalg eaqptkslee nkslkeqkkl ndlcflkrll qeiktcwnki lmgtkeh |

In some embodiments, the immune cell effector domain may be derived from IL-15. The amino acid sequence of a representative IL-15 is set forth below (SEQ ID NO: 22):

| | |
|---|---|
| 1 | mriskphlrs isiqcylcll lnshflteag ihvfilgcfs aglpkteanw vnvisdlkki |
| 61 | edliqsmhid atlytesdvh psckvtamkc fllelqvisl esgdasihdt venliilann |
| 121 | slssngnvte sgckeceele eknikeflqs fvhivqmfin ts |

In some embodiments, the immune cell effector domain may be derived from IL-18. The amino acid sequence of a representative IL-18 is set forth below (SEQ ID NO: 23):

| | |
|---|---|
| 1 | maaepvednc infvamkfid ntlyfiaedd enlesdyfgk lesklsvirn lndqvlfidq |
| 61 | gnrplfedmt dsdcrdnapr tifiismykd sqprgmavti svkcekistl scenkiisfk |
| 121 | emnppdnikd tksdiiffqr svpghdnkmg fesssyegyf lacekerdlf klilkkedel |
| 181 | gdrsimftvq ned |

In some embodiments, the immune cell effector domain may be derived from IL-21. The amino acid sequence of a representative IL-21 is set forth below (SEQ ID NO: 24):

| | |
|---|---|
| 1 | mrsspgnmer iviclmvifl gtlvhksssq gqdrhmirmr qlidivdqlk nyvndlvpef |
| 61 | lpapedvetn cewsafscfq kaqlksantg nneriinvsi kklkrkppst nagrrqkhrl |
| 121 | tcpscdsyek kppkeflerf ksllqkmihq hlssrthgse ds |

In some embodiments, the immune cell effector domain may be derived from IL-27. The amino acid sequence of a representative IL-27 is set forth below (SEQ ID NO: 25):

| | |
|---|---|
| 1 | mgqtagdlgw rlsllllpll lvqagvwgfp rppgrpqlsl qelrreftvs lhlarkllse |
| 61 | vrggahrfae shlpgvnlyl lplgeqlpdv sltfqawrrl sdperlcfis ttlqpfhall |
| 121 | gglgtqgrwt nmermqlwam rldlrdlqrh lrfqvlaagf nlpeeeeeee eeeeeerkgl |
| 181 | lpgalgsalq gpaqvswpql lstyrllhsl elvlsravre llllskaghs vwplgfptls |
| 241 | pqp |

In some embodiments, the immune cell effector domain is an immune cell-activating variant of a cytokine. In some embodiments, the immune cell effector domain is neoleukin-2/15 (Neo-2/15), which binds to the IL-2R-β, having the amino acid sequence set forth below (SEQ ID NO: 26).

| | |
|---|---|
| 1 | gshmpkkkiq lhaehalyda lmilnivktn sppaeekled yafnfelile eiarlfesgd |
| 61 | qkdeaekakr mkewmkrikt tasedeqeem anaiitilqs wifs |

In some embodiments, the CAR-engager contains Neo-2/15 two immune cell effector domains, each having the amino acid sequence of SEQ ID NO: 26.

In some embodiments, the immune cell effector domain may be derived from 4-1BBL. 4-1BBL is also known as TNF ligand superfamily member 9 (TNFSF9). The amino acid sequence of a representative 4-1BBL is provided at NCBI Accession No. NP_003802, incorporated herein by reference. In some embodiments, the immune cell effector domain may be derived from the extracellular domain of 4-1BBL. In some embodiments, the immune cell effector domain contains a portion of the extracellular domain of 4-1BBL, having the amino acid sequence set forth below (SEQ ID NO: 27):

| | |
|---|---|
| 1 | dpaglldlrq gmfaqlvaqn vllidgplsw ysdpglagvs ltgglsyked tkelvvakag |
| 61 | vyyvffqlel rrvvagegsg svslalhlqp lrsaagaaal altvdlppas searnsafgf |
| 121 | qgrllhlsag qrlgvhlhte ararhawqlt qgatvlglfr vtpeipa |

In some embodiments, the CAR-engager contains three immune cell effector domains, e.g., wherein all of the first, the second, and the third immune cell effector domains are the extracellular domain of 4-1BBL, each having the amino acid sequence of SEQ ID NO: 27.

In some embodiments, the immune cell effector domain may be a fragment, e.g., a single-chain variable antibody fragment (scFv), that binds and activates the CAR immune cell. In some embodiments, the immune cell effector domain is an scFv that binds to 4-1BB, CD2, CD27, CD28, CD30 (TNFRSF8), CD40L, CD226, CTLA4, GITR, IL-2R, LIGHT, OX40, PD-1, TIM2, SLAM, or TIM1.

In some embodiments, the immune cell effector domain is a scFv that binds CTLA4. In some embodiments, the immune cell effector domain is derived from a commercially available anti-CTLA4 antibody, antibody fragment, or derivative thereof, e.g., bavunalimab (formerly pavunalimab/XmAb 22841), botensilimab, cadonilimab, ipilimumab (Yervoy®), quavonlimab, tremelimumab (Imjudo®), volrustomig, vudalimab, or zalifrelimab. The amino acid sequences of representative heavy and light chains of antibodies that bind CTLA4 are set forth in Table 3.

TABLE 3
Amino acid Sequences of Representative anti-CTLA Antibody Heavy and Light Chains

botensilimab heavy chain (SEQ ID NO: 28)

| | |
|---|---|
| 1 | evqlvesggg lvkpggslrl scaasgftfs sysmnwvrqa pgkglewvss isssssyiyy |
| 61 | aesvkgrfti srdnaknsly lqmnslraed tavyycarvg lfgpfdiwgq gtlvtvssas |
| 121 | tkgpsvfpla psskstsggt aalgclvkdy fpepvtvswn sgaltsgvht fpavlqssgl |
| 181 | yslssvvtvp ssslgtqtyi cnvnhkpsnt kvdkrvepks cdkthtcppc papellggpd |
| 241 | vflfppkpkd tlmisrtpev tcvvvdvshe dpevkfnwyv dgvevhnakt kpreegynst |
| 301 | yrvvsvltvl hqdwingkey kckvsnkalp lpeektiska kgqprepqvy tlppsreemt |
| 361 | knqvsltclv kgfypsdiav ewesngqpen nykttppvld sdgsfflysk ltvdksrwqq |
| 421 | gnvfscsvmh ealhnhytqk slslspg |

botensilimab light chain (SEQ ID NO: 29)

| | |
|---|---|
| 1 | diqmtqspss lsasvgdrvt itcrasqdvs tavawyqqkp gkapklliys asflysgvps |
| 61 | rfsgsgsgtd ftltisslqp edfatyycqq ylyhpatfgq gtkveikrtv aapsvfifpp |
| 121 | sdeqlksgta svvcllnnfy preakvqwkv dnalqsgnsq esvteqdskd styslsstlt |
| 181 | lskadyekhk vyacevthqg 1sspvtksfn rgec |

ipilimumab heavy chain (SEQ ID NO: 30)

| | |
|---|---|
| 1 | qvqlvesggg wvqpgrslrl scaasgftfs sytmhwvrqa pgkglewvtf isydgnnkyy |
| 61 | adsvkgrfti srdnskntly lqmnslraed taiyycartg wlgpfdywgq gtlvtvssas |
| 121 | tkgpsvfpla psskstsggt aalgclvkdy fpepvtvswn sgaltsgvht fpavlqssgl |
| 181 | yslssvvtvp ssslgtqtyi cnvnhkpsnt kvdkrvepks cdkthtcppc papellggps |
| 241 | vflfppkpkd tlmisrtpev tcvvvdvshe dpevkfnwyv dgvevhnakt kpreeqynst |
| 301 | yrvvsvltvl hqdwingkey kckvsnkalp apiektiska kgqprepqvy tlppsrdelt |
| 361 | knqvsltclv kgfypsdiav ewesngqpen nykttppvld sdgsfflysk 1tvdksrwqq |
| | Slslspgk |

ipilimumab light chain (SEQ ID NO: 31)

| | |
|---|---|
| 1 | eivltqspgt lslspgerat lscrasqsvg ssylawyqqk pgqaprlliy gafsratgip |
| 61 | drfsgsgsgt dftltisrle pedfavyycq qygsspwtfg qgtkveikrt vaapsvfifp |
| 121 | psdeqlksgt asvvcllnnf ypreakvqwk vdnalqsgns qesvteqdsk dstyslsstl |
| 181 | tlskadyekh kvyacevthq glsspvtksf nrgec |

quavonlimab heavy chain (SEQ ID NO: 32)

| | |
|---|---|
| 1 | evqlvesggg lvqpggslrl scaasgftfs dnwmnwvrqa pgkglewlaq irnkpynyet |
| 61 | yysasvkgrf tisrddskns vylqmnslkt edtgvyycta qfaywgqgtl vtvssastkg |
| 121 | psvfplapss kstsggtaal gclvkdyfpe pvtvswnsga ltsgvhtfpa vlqssglysl |
| 181 | ssvvtvpsss lgtqtyicnv nhkpsntkvd kkvepkscdk thtcppcpap ellggpsvfl |
| 241 | fppkpkdtlm isrtpevtcv vvdvshedpe vkfnwyvdgv evhnaktkpr eeqynstyrv |
| 301 | vsvltvlhqd wlngkeykck vsnkalpapi ektiskakgq prepqvytlp psrdeltknq |
| 361 | vsltclvkgf ypsdiavewe sngqpennyk ttppvldsdg sfflyskltv dksrwqqgnv |
| 421 | fscsvmheal hnhytqksls lspg |

quavonlimab light chain (SEQ ID NO: 33)

| | |
|---|---|
| 1 | diqmtqspss lsasvgdrvt itcrtseniy gglnwyqrkp gkspklliyg atnlasgvss |
| 61 | rfsgsgsgtd ytltisslqp edvatyycqn vlrspftfgs gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

tremelimumab heavy chain (SEQ ID NO: 34)

| | |
|---|---|
| 1 | qvqlvesggg vvqpgrslrl scaasgftfs sygmhwvrqa pgkglewvav iwydgsnkyy |
| 61 | adsvkgrfti srdnskntly lqmnslraed tavyycardp rgatlyyyyy gmdvwgqgtt |
| 121 | vtvssastkg psvfplapcs rstsestaal gclvkdyfpe pvtvswnsga ltsgvhtfpa |
| 181 | vlqssglysl ssvvtvpssn fgtqtytonv dhkpsntkvd ktverkccve cppcpappva |
| 241 | gpsvflfppk pkdtlmisrt pevtcvvvdv shedpevqfn wyvdgvevhn aktkpreeqf |
| 301 | nstfrvvsvl tvvhqdwing keykckvsnk glpapiekti sktkgqprep qvytlppsre |
| 361 | emtknqvslt clvkgfypsd iavewesngq pennykttpp mldsdgsffl yskltvdksr |
| 421 | wqqgnvfscs vmhealhnhy tqkslslspg k |

tremelimumab light chain (SEQ ID NO: 35)

| | |
|---|---|
| 1 | diqmtqspss lsasvgdrvt itcrasqsin syldwyqqkp gkapklliya asslqsgvps |
| 61 | rfsgsgsgtd ftltisslqp edfatyycqq yystpftfgp gtkveikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

In some embodiments, the immune cell effector domain contains the VL having the amino acid sequence set forth below (SEQ ID NO 36):
1 eivltgspgt lslspgerat lscragsvsr ylgwyqqkpg qaprilliyga stratgipdr
61 fsgsgsgtdf tititriepe dfavyycqqy gsspwtfgqg tkveik

In some embodiments, the immune cell effector domain contains the VH having the amino acid sequence set forth below (SEQ ID NO 37):
1 evqlvesggg lvkpggslrl scaasgftfs sysmnwvrka pgkglewvss isssssyiyy
61 aesvkgrfti srdnaknsly lqmnslraed tavyycarvg lfgpfdiwgq gtlvtvss

In some embodiments, the immune cell effector domain binds OX4p. In some embodiments, the immune cell effector domain is derived from a commercially available anti-OX40 antibody, antibody fragment (e.g., scFv), or derivative thereof, e.g., tavolimab, or vonlerolizumab (Pogalizumab; MOXR 0916). The amino acid sequences of representative heavy and light chains of which are set forth in Table 4.

TABLE 4
Amino acid Sequences of Representative anti-OX40 Antibody Heavy and Light Chains

tavolimab heavy chain (SEQ ID NO: 38)

| | |
|---|---|
| 1 | qvqlqesgpg lvkpsqtlsl tcavyggsfs sgywnwirkh pgkgleyigy isyngityhn |
| 61 | pslksritin rdtsknqysl qlnsvtpedt avyycaryky dydgghamdy wgqgtlvtvs |
| 121 | sastkgpsvf plapssksts ggtaalgclv kdyfpepvtv swnsgaltsg vhtfpavlqs |
| 181 | sglyslssvv tvpssslgtq tyicnvnhkp sntkvdkrve pkscdkthtc ppcpapellg |
| 241 | gpsvflfppk pkdtlmisrt pevtcvvvdv shedpevkfn wyvdgvevhn aktkpreeqy |
| 301 | nstyrvvsvl tvlhqdwlng keykckvsnk alpapiekti skakgqprep qvytlppsre |
| 361 | emtknqvslt clvkgfypsd iavewesngq pennykttpp vldsdgsffl yskltvdksr |
| 421 | wqqgnvfscs vmhealhnhy tqkslslspg k |

tavolimab light chain (SEQ ID NO: 39)

| | |
|---|---|
| 1 | diqmtqspss lsasvgdrvt itcrasqdis nylnwyqqkp gkapklliyy tsklhsgvps |
| 61 | rfsgsgsgtd ytltisslqp edfatyycqq gsalpwtfgq gtkveikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

vonlerolizumab heavy chain (SEQ ID NO: 40)

| | |
|---|---|
| 1 | evqlvqsgae vkkpgasvkv sckasgytft dsymswvrqa pgqglewigd mypdngdssy |
| 61 | nqkfrervti trdtststay lelsslrsed tavyycvlap rwyfsvwgqg tlvtvssast |
| 121 | kgpsvfplap sskstsggta algclvkdyf pepvtvswns galtsgvhtf pavlqssgly |
| 181 | slssvvtvps sslgtqtyic nvnhkpsntk vdkkvepksc dkthtcppcp apellggpsv |
| 241 | flfppkpkdt lmisrtpevt cvvvdvshed pevkfnwyvd gvevhnaktk preeqynsty |
| 301 | rvvsvltvlh qdwingkeyk ckvsnkalpa piektiskak gqprepqvyt lppsreemtk |
| 361 | nqvsltclvk gfypsdiave wesngqpenn ykttppvlds dgsfflyskl tvdksrwqqg |
| 421 | nvfscsvmhe alhnhytqks lslspgk |

vonlerolizumab light chain (SEQ ID NO: 41)

| | |
|---|---|
| 1 | diqmtqspss lsasvgdrvt itcrasqdis nylnwyqqkp gkapklliyy tsrlrsgvps |
| 61 | rfsgsgsgtd ftltisslqp edfatyycqq ghtlpptfgq gtkveikrtv aapsvfifpp |
| 121 | sdeqlksgta svvcllnnfy preakvqwkv dnalqsgnsq esvteqdskd styslsstlt |
| 181 | 1skadyekhk vyacevthqg lsspvtksfn rgec |

In some embodiments, the immune cell effector domain contains the VL having the amino acid sequence set forth below (SEQ ID NO 42):

| | |
|---|---|
| 1 | diqmtqspss lsasvgdrvt itcrasqdis nylnwyqqkp gkapklliyy tsrlrsgvps |
| 61 | rfsgsgsgtd ftltisslqp edfatyycqq ghtlpptfgq gtkveik |

In some embodiments, the immune cell effector domain contains the VH having the amino acid sequence set forth below (SEQ ID NO 43):

| | |
|---|---|
| 1 | evqlvqsgae vkkpgasvkv sckasgytft dsymswvra pgqglewigd mypdngdssy |
| 61 | nqkfrervti trdtststay lelsslrsed tavyycvlap rwyfsvwgqg tlvtvss |

In some embodiments, the immune cell effector domain binds PD-1. In some embodiments, the immune cell effector domain is derived from a commercially available anti-PD-1 antibody, antibody fragment (e.g., scFv), or derivative thereof, e.g., atezolizumab, avelumab, bintrafusp alfa, cosibelimab, danburstotug, durvalumab (Imfinzi®), inbakicept, lodapolimab, pimivalimab, or socazolimab. The amino acid sequences ofrepresentative heavy and light chains of which are set forth in Table 5.

TABLE 5
Amino Acid Sequences of Representative anti-PD-1 Antibody Heavy and Light Chains

atezolizumab heavy chain (SEQ ID NO: 44)

| | |
|---|---|
| 1 | evqllesggg lvqpggslrl scaasgftfs syimmwvrqa pgkglewvss iypsggitfy |
| 61 | adtvkgrfti srdnskntly lqmnslraed tavyycarik lgtvttvdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkkvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsrde |
| 361 | ltknqvsltc lvkgfypsdi avewesngqp ennykttppv ldsdgsffly skltvdksrw |
| 421 | qqgnvfscsv mhealhnhyt qkslslspgk |

atezolizumab light chain (SEQ ID NO: 45)

| | |
|---|---|
| 1 | qsaltqpasv sgspgqsiti sctgtssdvg gynyvswyqq hpgkapklmi ydvsnrpsgv |
| 61 | snrfsgsksg ntasltisgl qaedeadyyc ssytssstrv fgtgtkvtvl gqpkanptvt |
| 121 | lfppsseelq ankatlvcli sdfypgavtv awkadgspvk agvettkpsk qsnnkyaass |
| 181 | ylsltpeqwk shrsyscqvt hegstvektv aptecs |

avelumab heavy chain (SEQ ID NO: 46)

| | |
|---|---|
| 1 | evqllesggg lvqpggslrl scaasgftfs syimmwvrqa pgkglewvss iypsggitfy |
| 61 | adtvkgrfti srdnskntly lqmnslraed tavyycarik lgtvttvdyw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkkvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsrde |
| 361 | ltknqvsltc lvkgfypsdi avewesngqp ennykttppv ldsdgsffly skltvdksrw |
| 421 | qqgnvfscsv mhealhnhyt qkslslspgk |

avelumab light chain (SEQ ID NO: 47)

| | |
|---|---|
| 1 | qsaltqpasv sgspgqsiti sctgtssdvg gynyvswyqq hpgkapklmi ydvsnrpsgv |
| 61 | snrfsgsksg ntasltisgl qaedeadyyc ssytssstrv fgtgtkvtvl gqpkanptvt |
| 121 | lfppsseelq ankatlvcli sdfypgavtv awkadgspvk agvettkpsk qsnnkyaass |
| 181 | ylsltpeqwk shrsyscqvt hegstvektv aptecs |

durvalumab heavy chain (SEQ ID NO: 48)

| | |
|---|---|
| 1 | evqlvesggg lvqpggslrl scaasgftfs rywmswvrqa pgkglewvan ikqdgsekyy |
| 61 | vdsvkgrfti srdnaknsly lqmnslraed tavyycareg gwfgelafdy wgqgtlvtvs |
| 121 | sastkgpsvf plapssksts ggtaalgclv kdyfpepvtv swnsgaltsg vhtfpavlqs |
| 181 | sglyslssvv tvpssslgtq tyicnvnhkp sntkvdkrve pkscdkthtc ppcpapefeg |
| 241 | gpsvflfppk pkdtlmisrt pevtcvvvdv shedpevkfn wyvdgvevhn aktkpreegy |
| 301 | nstyrvvsvl tvlhqdwlng keykckvsnk alpasiekti skakgqprep qvytlppsre |
| 361 | emtknqvslt clvkgfypsd iavewesngq pennykttpp vldsdgsffl yskltvdksr |
| 421 | wqqgnvfscs vmhealhnhy tqkslslspg k |

durvalumab light chain (SEQ ID NO: 49)

| | |
|---|---|
| 1 | eivltqspgt lslspgerat lscrasqrvs ssylawyqqk pgqaprlliy dassratgip |
| 61 | drfsgsgsgt dftltisrle pedfavyycq qygslpwtfg qgtkveikrt vaapsvfifp |
| 121 | psdeqlksgt asvvcllnnf ypreakvqwk vdnalqsgns qesvteqdsk dstyslsstl |
| 181 | tlskadyekh kvyacevthq lsspvtksf nrgec |

pimivalimab heavy chain (SEQ ID NO: 50)

| | |
|---|---|
| 1 | qvqlvqsgae vkkpgasvkv sckasgytfp syymhwvrqa pgqglewmgi inpeggstay |
| 61 | aqkfqgrvtm trdtststvy melsslrsed tavyycargg tyydytywgq gtlvtvssas |
| 121 | tkgpsvfpla pcsrstsest aalgclvkdy fpepvtvswn sgaltsgvht fpavlqssgl |
| 181 | yslssvvtvp ssslgtktyt cnvdhkpsnt kvdkrvesky gppcppcpap eflggpsvfl |
| 241 | fppkpkdtlm isrtpevtcv vvdvsqedpe vqfnwyvdgv evhnaktkpr eeqfnstyrv |
| 301 | vsvltvlhqd wlngkeykck vsnkglpssi ektiskakgq prepqvytlp psqeemtknq |
| 361 | vsltclvkgf ypsdiavewe sngqpennyk ttppvldsdg sfflysrltv dksrwqegnv |
| 421 | fscsvmheal hnhytqksls lslg |

pimivalimablight chain (SEQ ID NO: 51)

| | |
|---|---|
| 1 | diqmtqspst lsasvgdrvt itcrasqsis swlawyqqkp gkapklliye asslesgvps |
| 61 | rfsgsgsgte ftltisslqp ddfatyycqq ynsfpptfgg gtkveikrtv aapsvfifpp |
| 121 | sdeqlksgta svvcllnnfy preakvqwkv dnalqsgnsq esvteqdskd styslsstlt |
| 181 | 1skadyekhk vyacevthqg lsspvtksfn rgec |

In some embodiments, the immune cell effector domain contains the VL having the amino acid sequence set forth below (SEQ ID NO 52):

| | |
|---|---|
| 1 | eivmtqspat lsvspgerat lscrasqsvs snlawyqqkp gqaprlliyg astratgipa |
| 61 | rfsgsgsgte ftltisslqs edfavyycqq ynnwprtfgq gtkveik |

In some embodiments, the immune cell effector domain contains the VH having the amino acid sequence set forth below (SEQ ID NO 53):

| | |
|---|---|
| 1 | qvqlvesggg vvqpgrslrl scaasgftfs sygmhwvrqa pgkglewvav iwydgsnkyy |
| 61 | adsvmgrfti srdnskntly lqmnslraed tavyycasng dhwgqgtlvt vss |

In some embodiments, the immune cell effector domain is an immune cell-inhibiting moiety, representative types of which include immune cell inhibiting cytokines and immune cell-inhibiting variants and fragments thereof. Immune cell inhibiting moieties repress or block immune cell activity and function. In some embodiments, the immune cell-inhibiting moiety may be derived from CD80, CD86, CD112, CD155, CD276 (B37-1r13), Ceacam-1, FGL1, galectin-3, ITLA-E, HVEM, PD-L1, PD-L2, VISTA, or VTCN1 (B37-H4). The amino acid sequences of representative immune cell-inhibiting proteins from which the immune cell effector domain may be derived are provided at the NCBI Accession numbers set forth in Table 6, and are incorporated herein by reference.

TABLE 6
Gene Name, Symbols, and NCBI Accession Numbers of Immune Cell-Inhibiting Proteins

| Gene Name | Gene Symbols | Protein Accession No(s). |
|---|---|---|
| CD80 molecule | B7, B7-1, B7.1, BB1, | NP_005182 |
| | CD28LG, CD28LG1, CD80, | |
| | LAB7 | |
| CD86 molecule | B7-2, B7.2, B70, BU63, | NP_001193853, NP_001193854, |
| | CD28LG2, CD86, FUN-1, | NP_008820, NP_787058, |
| | LAB72 | NP_795711 |
| Nectin cell adhesion | CD112, HVEB, HveB, | XP_047295125, NP_001036189, |
| molecule 2 | NECTIN2, Nectin-2, PRR2, | NP_002847 |
| | PVRL2, PVRR2 | |
| Pvr cell adhesion | CD155, HVED, NECL-5, | NP_001129240, NP_001129241, |
| molecule | NECL5, Necl-5, PVR, PVS, | NP_001129242, NP_006496 |
| | TAGE4, Tage4 | |
| CD200 molecule | CD200, MOX1, MOX2, | NP_001004196, NP_001305755, |
| | MRC, OX-2 | NP_001305757, NP_001305759, |
| | | NP_001352780, NP_001352781, |
| | | NP_001352782, NP_001352783, |
| | | NP_001352784, NP_005935 |
| CD276 molecule | 4Ig-B7-H3, B7-H3, B7H3, | XP_005254757, XP_011520397, |
| | B7RP-2, CD276 | XP_016878127, XP_047289103, |
| | | XP_047289104, NP_001019907, |
| | | NP_001316557, NP_001316558, |
| | | NP_079516 |
| CEA cell adhesion | BGP, BGP-1, BGP1, BGPI, | XP_011525508, NP_001020083, |
| molecule 1 | CD66a, CEACAM1 | NP_001171742, NP_001171744, |
| | | NP_001171745, NP_001192273, |
| | | NP_001703 |
| Fibrinogen like 1 | FGL1, HFREP-1, HFREP1, | XP_047277533, NP_004458, |
| | HP-041, HPS, LFIRE-1, | NP_671736, NP_963846, |
| | LFIRE1 | NP_963847 |
| Galectin 3 | CBP 35, CBP35, GAL3, | XP_047287323, XP_047287324, |
| | GALBP, GALIG, Gal-3, L- | XP_047287325, NP_001344607, |
| | 31, L31, LGALS2, LGALS3, | NP_002297 |
| | MAC-2, MAC2 | |
| Herv-h ltr-associating 2 | B7-H5, B7-H7, B7H7, B7y, | XP_005247137, XP_011510664, |
| | HHLA2 | XP_011510665, XP_011510666, |
| | | XP_011510669, XP_024309094, |
| | | XP_047303324, XP_047303325, |
| | | NP_001269485, NP_001269486, |
| | | NP_001269487, NP_001269488, |
| | | NP_001357173, NP_009003 |
| Major histocompatibility | HLA-6.2, HLA-E, HLAE, | XP_016866296, NP_005507 |
| complex, class i, e | QA1 | |
| TNF receptor | ATAR, CD270, HVEA, | XP_006711082, XP_011540685, |
| superfamily member 14 | HVEM, HveA, LIGHTR, | XP_047289368, XP_047289369, |
| | TNFRSF14, TR2 | XP_047289370, XP_047289372, |
| | | XP_047289374, XP_047289375, |
| | | XP_047289377, XP_047289378, |
| | | XP_047289379, NP_001284534, |
| | | NP_003811 |
| Programmed cell death 1 | B7-H, B7-H1, B7H1, CD274, | XP_047279218, NP_001254635, |
| ligand 1 | HPD-L1, PD-L1, PDCD1L1, | NP_001300958, NP_054862 |
| | PDCD1LG1, PDL1 | |
| Programmed cell death 1 | B7-DC, B7DC, Btdc, CD273, | XP_005251657, NP_079515 |
| ligand 2 | PD-L2, PDCD1L2, | |
| | PDCDILG2, PDL2 | |
| Inducible T cell | B7-H2, B7H2, B7RP-1, | XP_011527816, XP_011527818, |
| costimulator ligand | B7RP1, B7h, CD275, GL50, | XP_047296685, XP_047296686, |
| | ICOS-L, ICOSL, ICOSLG, | XP_047296687, XP_047296688, |
| | KIAA0653, LICOS | NP_001269979, NP_001269980, |
| | | NP_001269981, NP_001352688, |
| | | NP_001382847, NP_056074 |
| V-set domain containing | B7-H4, B7H4, B7S1, B7X, | XP_011540445, XP_016857824, |
| T cell activation inhibitor 1 | B7h.5, B7x, FLJ22418, | NP_001240778, NP_001240779, |
| | PRO1291, VCTNI, VTCN1 | NP_078902 |
| V-set immunoregulatory | B7-H5, B7H5, C10orf54, | NP_071436 |
| receptor | DD1alpha, Dies1, GI24, PD- | |
| | 1H, PP2135, SISP1, Sisp-1, | |
| | VISTA, VSIR | |

In some embodiments, the immune cell effector domain is the extracellular domain of CD80. The amino acid sequence of a representative CD80 extracellular domain is set forth below (SEQ ID NO: 54):

| | |
|---|---|
| 1 | vihvtkevke vatlscghnv sveelaqtri ywqkekkmvl tmmsgdmniw peyknrtifd |
| 61 | itnnlsivil alrpsdegty ecvvlkyekd afkrehlaev tlsvkadfpt psisdfeipt |
| 121 | snirriicst sggfpephls wlengeelna inttvsqdpe telyavsskl dfnmttnhsf |
| 181 | mclikyghlr vnqtfnwntt kqehfpdn |

In some embodiments, the immune cel effector domain is the extracellular domain of CD86. The amino acid sequence of a representative CD86 extracellular domain is set forth below (SEQ ID NO: 55):

| | |
|---|---|
| 1 | aplkiqayfn etadlpcqfa nsqnqslsel vvfwqdqenl vlnevylgke kfdsvhskym |
| 61 | grtsfdsdsw tlrlhnlqik dkglyqciih hkkptgmiri hqmnselsvl anfsqpeivp |
| 121 | isnitenvyi nltcssihgy pepkkmsvll rtknstieyd gvmqksqdnv telydvsisl |
| 181 | svsfpdvtsn mtifciletd ktrllsspfs ieledpqppp dhip |

In some embodiments, the immune cell effector domain is the extracellular domain of CD155 (nectin-5; PVR). The amino acid sequence of a representative CD155 extracellular domain is set forth below (SEQ ID NO: 56):

| | |
|---|---|
| 1 | wpppgtgdvv vqaptqvpgf lgdsvtlpcy lqvpnmevth vsqltwarhg esgsmavfhq |
| 61 | tqgpsysesk rlefvaarlg aelrnaslrm fglrvedegn ytclfvtfpq gsrsvdiwlr |
| 121 | vlakpqntae vqkvaltgep vpmarcvstg grppaqitwh sdlggmpnts qvpgflsgtv |
| 181 | tvtslwilvp ssqvdgknvt ckvehesfek pqlltvnltv yyppevsisg ydnnwylgqn |
| 241 | eatltcdars npeptgynws ttmgplppfa vaqgaqllir pvdkpinttl icnvtnalga |
| 301 | rqaeltvqvk egppsehsgi srn |

In some embodiments, the immune cell effector domain is the extracellular domain of CD276 (B7-H3). The amino acid sequence of a representative CD276 extracellular domain is set forth below (SEQ ID NO: 57):

| | |
|---|---|
| 1 | levqvpedpv valvgtdatl cosfspepgf slaglnliwg ltdtkqlvhs faegqdqgsa |
| 61 | yanrtalfpd llaqgnasir lqrvrvadeg sftcfvsird fgsaavslqv aapyskpsmt |
| 121 | lepnkdlrpg dtvtitossy ggypeaevfw qdgqgvoltg nvttsqmane qglfdvhsil |
| 181 | rvvlgangty sclvrnpvlq qdahssvtit porsptgave vqvpedpvva lvgtdatlrc |
| 241 | sfspepgfsl aqinliwglt dtkqlvhsft egrdqgsaya nrtalfpdll aqgnaslrlq |
| 301 | rvrvadegsf tofvsirdfg saavslqvaa pyskpsmtle pnkdlrpgdt vtitcssyrg |
| 361 | ypeaevfwqd gqgvpltgnv ttsomaneqg lfdvhsvirv vlgangtysc lvrnpvlqqd |
| 421 | ahgsvtitgq pmtfppea |

In some embodiments, the immune cell effector domain is the extracellular domain of Ceacam-1. The amino acid sequence of a representative Ceacma-1 extracellular domain is set forth below (SEQ ID NO: 58):

| | |
|---|---|
| 1 | kltiesmpls vaegkevlll vhnlpqhlfg yswykgervd gnslivgyvi gtqqatpgaa |
| 61 | ysgretiytn aslliqnvtq ndigfytlqv iksdlvneea tgqfhvyqen apglpvgava |
| 121 | q |

In some embodiments, the immune cell effector domain is the extracellular domain of FGL1. The amino acid sequence of a representative FGL1 extracellular domain is set forth below (SEQ ID NO: 59):

| | |
|---|---|
| 1 | makvfsfilv ttaltmgrei saledcaqeq mrlraqvrll etrvkqqqvk ikqllqenev |
| 61 | qfldkgdent vidlgskrqy adcseifndg yklsgfykik plqspaefsv ycdmsdgggw |
| 121 | tviqrrsdgs enfnrgwkdy engfgnfvqk hgeywlgnkn lhflttqedy tlkidladfe |
| 181 | knsryaqykn fkvgdeknfy elnigeysgt agdslagnfh pevqwwashq rmkfstwdrd |
| 241 | hdnyegncae edqsgwwfnr chsanlngvy ysgpytaktd ngivwytwhg wwyslksvvm |
| 301 | kirpndfipn vi |

In some embodiments, the immune cell effector domain is the extracellular domain of galectin-3. The amino acid sequence of a representative galecin-3 extracellular domain is set forth below (SEQ ID NO: 60):

| | |
|---|---|
| 1 | madnfslhda lsgsgnpnpq gwpgawgnqp agaggypgas ypgaypgqap pgaypgqapp |
| 61 | gaypgapgay pgapapgvyp gppsgpgayp ssgqpsatga ypatgpygap agplivpynl |
| 121 | plpggvvprm litilgtvkp nanrialdfq rgndvafhfn prfnennrrv ivontkldnn |
| 181 | wgreerqsvf pfesgkpfki qvlvepdhfk vavndahllq ynhrvkklne isklgisgdi |
| 241 | dltsasytmi |

In some embodiments, the immune cell effector domain is the extracellular domain oP, HLA-E. The amino acid sequence of a representative HLA-E extracellular domain is set forth below (SEQ ID NO: 61):

| | |
|---|---|
| 1 | gshslkyfht svsrpgrgep rfisvgyvdd tqfvrfdnda asprmvprap wmeqegseyw |
| 61 | dretrsardt aqifrvnlrt lrgyynqsea gshtlqwmhg celgpdgrfl rgyeqfaydg |
| 121 | kdyltlnedl rswtavdtaa qiseqksnda seaehqrayl edtcvewlhk ylekgketll |
| 181 | hleppkthvt hhpisdheat lrcwalgfyp aeitltwqqd geghtqdtel vetrpagdgt |
| 241 | fqkwaavvvp sgeeqrytch vqheglpepv tlrwkpasqp tipi |

In some embodiments, the immune cell effector domain is the extracellular domain of HVEM (CD270). The amino acid sequence set of a representative HVEM extracellular domain is forth below (SEQ ID NO: 62):

| | |
|---|---|
| 1 | lpsckedeyp vgseccpkcs pgyrvkeacg eltgtvcepc ppgtyiahln glskclqcqm |
| 61 | cdpamglras rncsrtenav cgcspghfci vqdgdhcaac rayatsspgq rvqkggtesq |
| 121 | dtlcqncppg tfspngtlee cqhqtkcswl vtkagagtss shwv |

In some embodiments, the immune cell effector domain the extracellular domain of nectin-2 (CD112, HVEB). The amino acid sequence of a representative nectin-2 extracellular domain is set forth below (SEQ ID NO: 63):

| | |
|---|---|
| 1 | qdvrvqvlpe vrgqlggtve lpchllppvp glyislvtwq rpdapanhqn vaafhpkmgp |
| 61 | sfpspkpgse rlsfvsakqs tgqdteaelq datlalhglt vedegnytce fatfpkgsvr |
| 121 | gmtwlrviak pknqaeaqkv tfsqdpttva lciskegrpp ariswlssld weaketqvsg |
| 181 | tlagtvtvts rftlvpsgra dgvtvtckve hesfeepali pvtlsvrypp evsisgyddn |
| 241 | wylgrtdatl scdvrsnpep tgydwsttsg tfptsavagg sqlvihavds lfnttfvctv |
| 301 | tnavgmgrae qvifvretpn tagagatgg |

In some embodiments, the immune cell effector domain is the extracellular domain of PD-L1. The amino acid sequence of a representative PD-Llextracellular domain is set forth below (SEQ ID NO: 64):

| | |
|---|---|
| 1 | ftvtvpkdly vveygsnmti eckfpvekql dlaalivywe medkniiqfv hgeedlkvqh |
| 61 | ssyrqrarll kdqlslgnaa lqitdvklqd agvyrcmisy ggadykritv kvnapynkin |
| 121 | qrilvvdpvt seheltcqae gypkaeviwt ssdhqvlsgk ttttnskree klfnvtstlr |
| 181 | intttneify ctfrrldpee nhtaelvipe lplahppner |

In some embodiments, the immune cell effector domain is the extracellular domain of PD-L2. The amino acid sequence set of a representative PD-L2 extracellular domain is forth below (SEQ ID NO: 65):

| | |
|---|---|
| 1 | lftvtvpkel yiiehgsnvt lecnfdtgsh vnlgaitasl qkvendtsph reratlleeq |
| 61 | lplgkasfhi pqvqvrdegq yqciiiygva wdykyltlkv kasyrkinth ilkvpetdev |
| 121 | eltcqatgyp laevswpnvs vpantshsrt peglyqvtsv lrlkpppgrn fscvfwnthv |
| 181 | reltlasidl qsqmeprthp t |

In some embodiments, the immune cell effector domain is the extracellular domain of VTCN1 (B7-H4). The amino acid sequence of a representative VTCN1 is set forth below (SEQ ID NO: 66):

| | |
|---|---|
| 1 | liigfgisgr hsitvttvas agnigedgil sctfepdikl sdiviqwlke gvlglvhefk |
| 61 | egkdelseqd emfrgrtavf adqvivgnas lrlknvqltd agtykcyiit skgkgnanle |
| 121 | yktgafsmpe vnvdynasse tlrceaprwf poptvvwasq vdqganfsev sntsfelnse |
| 181 | nvtmkvvsvl ynvtinntys cmiendiaka tgdikvtese ikrrshlqll nskas |

### Dimerization Domain

In some embodiments, the CAR-engager further includes a dimerization domain. In these cases, the CAR-engager forms and is administered in the form of a homodimer or a homo-multimer. The homodimer thus contains two CAR-engager entities. The order of the ectodomain, the immune effector domain and the dimerization domain is not critical. In some embodiments, the dimerization domain is disposed between an ectodomain and an immune cell effector domain.

In some embodiments, the CAR-engager is in the form of a heterodimer, which contains a first entity containing an ectodomain of an antigen present on a cancer cell connected to a first dimerization domain and a second entity containing an immune cell effector domain connected to a second dimerization domain. In these embodiments, the first and second dimerization domains dimerize the first and second entities to form a heterodimer.

In some embodiments, the first and second dimerization domains contain a knob-in-hole configuration. One of the dimerization domains contains a protuberance (knob) and the other dimerization domain contains a cavity (hole) that is sterically compensatory to the protuberance, where the tertiary structure of the protuberance is positionable within the tertiary structure of the cavity. Dimerization domains with knob-in-hole configurations may have directed amino acid mutations where the protuberance is an amino acid that has a larger side chain volume than present on a dimerization domain derived from a natural source (e.g., IgA, IgD, IgG, IgM, or IgE) and the cavity is an amino acid that has a smaller side chain volume than present on a dimerization domain derived from a natural source.

In some embodiments, the protuberance is an amino acid change from a threonine (T) to a lysine (K) and the corresponding cavity is an amino acid change from a leucine (L) to an aspartic acid (D) or a lysine (K). In some embodiments, the first dimerization domain contains two amino acid substitutions, for example, a threonine (T) to a lysine (K) and a leucine (L) to a lysine (K), while the second dimerization domain contains a leucine (L) to an aspartic acid (D) or a glutamic acid (E) and a tyrosine (Y) to a glutamic acid (E) or aspartic acid (D).

In some embodiments, the knob-in-hole dimerization domains are based on opposed charges. In some embodiments, the first dimerization domain contains a positively charged amino acid and the second dimerization domain contains a negatively charged amino acid sterically opposable to the positively charged amino acid on the first dimerization domain.

Additional protuberance and cavity arrangements are known in the art. See, e.g., U.S. Pat. Nos. 5,821,333, 7,183,076, 8,642,745, 9,248,182, 9,309,311, 9,527,927, 9,562,109, 9,890,204, 10,138,303, and 11,168,344 and U.S. Patent Application Publications 2005/0079170, 2006/0025576, 2013/0089554, and 2014/0024111.

In some embodiments, the dimerization domains may be derived from IgA, IgD, IgG, IgM, or IgE. The first and the second dimerization domains may contain the same or different amino acid sequences, provided that they bind each other. In some embodiments, the first and the second dimerization domains are the IgG1 constant heavy (CH) 3 domain. The amino acid sequence of a representative IgG1 CH3 domain is set forth below (SEQ ID NO: 67):

| | |
|---|---|
| 1 | epkspksadk thtapqprep qvytlppsrd eltknqvslt clvkgfypsd iavewesngq |
| 61 | pennykttpp vldsdgsffl yskltvdksr wqqgnvfscs vmhealhnhy tqkslslspg |
| 121 | k |

In some embodiments, the first and the second dimerization domains are the IgG1 constant heavy CH2 domain. The amino acid sequence of a representative IgG1 CH2 domain is set forth below (SEQ ID NO: 68):

| | |
|---|---|
| 1 | pcpapellgg psvflfppkp kdtlmisrtp evtcvvvdvs hedpevkfnw yvdgvevhna |
| 61 | ktkpreeqyn styrvvsvlt vlhqdwlngk eykckvsnka lpapiektis kak |

In some embodiments, the first and the second dimerization domains are the IgG1 CH2 and CH3 domains. The CH2 and CH3 domains may be interconnected by a linker.

### Linkers

In some embodiments, the CAR-engager contains one or more linkers. A linker may provide flexibility in terms of allowing the ectodomain and the immune cell effector domains to bind to their respective cognate receptors on the CAR-expressing immune cell or steric spacing (i.e., a spacer) between the ectodomain and the immune cell effector domain.

A linker may be disposed between any two CAR-engager components (also referred to herein as domains, entities or moieties or portions) (e.g., the ectodomain and the immune cell effector domain).

A linker may be disposed between the dimerization domain and the adjacent domain. In some embodiments, a linker may be disposed between the dimerization domain and the immune cell effector domain. In some embodiments, the CAR-engager contains two linkers, where a first linker is disposed between the ectodomain and the dimerization domain, and a second linker is disposed between the dimerization domain and the immune cell effector domain.

In some embodiments, the linker comprises an amino acid having the sequence GGGX, GGGGX (SEQ ID NO: 69), or GSSGSX (SEQ ID NO: 70), where X is any nucleotide, typically either cysteine (C) or serine (S), or repeating sequence thereof. In some embodiments, the linker has the amino acid sequence GGGGS (SEQ ID NO: 71), GSPRG (SEQ ID NO: 72), GGGGSGGGGS (SEQ ID NO: 73), GGGGSGGGGSGGGGS (SEQ ID NO: 74), GGGGSGGGGSGGGGSGGGGS (SEQ ID NO: 75), GSPRGGGGSGGGGSGGGGS (SEQ ID NO: 76), GSTSGSGKPGSGEGSTKG (SEQ ID NO: 77), KESGSVSSEQLAQFRSLD (SEQ ID NO: 78), EGKSSGSGSESKST (SEQ ID NO: 79), or GSAGSAAGSGEF (SEQ ID NO: 80).

In some embodiments, the linker may be derived from IgA, IgD, IgE, IgG, or IgM. In some embodiments, the linker may be derived from the hinge region of CD3ζ, CD4, CD8a, CD28, IgG1, IgG2, or IgG4. Amino acid sequences of representative linkers are listed in Table 7.

TABLE 7
Amino acid Sequences of Representative Linkers

| Linker | Sequence |
|---|---|
| CD3 (SEQ ID NO: 81) | QSFGLLDPK |
| CD4 (SEQ ID NO: 82) | LSEGDKVKMDSRIQVLSRGVNQT |
| CD8α (SEQ ID NO: 83) | KPTTTPAPRPPTPAPTIASQPLSKRPEACRPAAGGAVHTRGLDFA |
| | CDIY |
| CD28 (SEQ ID NO: 84) | IEVMYPPPYLDNERSNGTIIHVKGKHLCPSPLFPGPSKP |
| IgG1 (SEQ ID NO: 85) | EPKSCDKTHTCPPCPAPELLGG |
| IgG2 (SEQ ID NO: 86) | ERKCCVECPPCPAPPAAA |
| IgG4 (SEQ ID NO: 87) | ESKYGPPCPPCPAPEFLGG |

In some embodiments, the CAR-engager is in the form of a fusion protein, where the components are linked by peptide bonds. In other embodiments, the CAR-engager contains proteinaceous entities interconnected by click chemistry, a chemical connection formed by a method of controlled protein ligation. The connection may be an azide-alkyne connection, an oxime or hydrazine connection, a tetrazine-transcyclooctene connection, an azide-nitrone connection, a thiol-alkene connection, an alkene-tetrazole connection, an alkene-tetrazine connection, an alkene-azide connection, a conjugated diene-alkene connection, or an isonitrile-tetrazine connection.

Additional controlled protein ligation chemistries, systems, and methods are known in the art. See, e.g., U.S. Pat. Nos. 7,375,234, 7,763,736, 8,101,238, 8,372,986, 8,394,914, 8,877,170, 8,927,682, 8,927,736, 9,302,997, 9,896,547, 11,028,185, 11,091,588, and 11,352,460 and U.S. Patent Application Publication 2009/0069561.

### Nucleic Acids

In another aspect, the present disclosure provides a nucleic acid that encodes a CAR-engager protein. The term “nucleic acid” as used herein refers to a polymer of nucleotides, each of which are organic molecules consisting of a nucleoside (a nucleobase and a five-carbon sugar) and a phosphate. The term nucleotide, unless specifically stated or obvious from context, includes nucleosides that have a ribose sugar (i.e., a ribonucleotide that forms ribonucleic acid, RNA) or a 2′-deoxyribose sugar (i.e., a deoxyribonucleotide that forms deoxyribonucleic acid, DNA). Nucleotides serve as the monomeric units of nucleic acid polymers or polynucleotides. The four nucleobases in DNA are guanine (G), adenine (A), cytosine (C) and thymine (T). The four nucleobases in RNA are guanine (G), adenine (A), cytosine (C) and uracil (U). Nucleic acids are linear chains of nucleotides (e.g., at least 3 nucleotides) chemically bonded by a series of ester linkages between the phosphoryl group of one nucleotide and the hydroxyl group of the sugar (i.e., ribose or 2′-deoxyribose) in the adjacent nucleotide.

In some embodiments, the CAR-engager is encoded by two nucleic acids, e.g., the ectodomain is encoded by a first nucleic acid and the immune cell effector domain is encoded by a second nucleic acid.

In some embodiments, nucleic acid encoding the CAR-engager includes a signal peptide-encoding nucleic acid disposed 5′ to the nucleic acid encoding the ectodomain. The term “signal peptide” as used herein refers to a short (e.g., 5-30 or 10-100 amino acids long) stretch of amino acids that directs the transport of the protein during translation. CAR-engagers containing a signal peptide will be secreted from the cell. Typically, the signal peptide is cleaved from the CAR-engager before secretion. The signal peptide may be connected to the nucleic acid encoding the ectodomain or the nucleic acid encoding the immune cell effector domain.

In some embodiments, the signal peptide may be derived from Ig-7-3 heavy chain (IGHG3), albumin, CD89, CD33, erythropoietin (EPO), IL-2, human or mouse Ig-kappa chain V-III (IgK VIII), tissue plasminogen activator (tA), or secreted alkaline phosphatase (SEAP). Signal peptides may also be synthetic (i.e., non-naturally occurring). Amino acid sequences of representative signal peptides are listed in Table 8.

TABLE 8
Amino acid Sequences of Representative Signal Peptides

| Signal peptide | Sequence |
|---|---|
| IGHG3 (SEQ ID NO: 88) | MKHLWFFLLLVAAPRWVLS |
| Albumin (SEQ ID NO: 89) | MKWVTFISLLFLFSSAYS |
| Synthetic, modified albumin (SEQ ID NO: 90) | MKWVTFISLLFLFSSSSRA |
| CD8α (SEQ ID NO: 91) | MALPVTALLLPLALLLHAARP |
| CD33 (SEQ ID NO: 92) | MPLLLLLPLLWAGALA |
| EPO (SEQ ID NO: 93) | MGVHECPAWLWLLLSLLSLPLGLPVLG |
| IL-2 (SEQ ID NO: 94) | MYRMQLLSCIALSLALVINS |
| Mouse IgK VIII (SEQ ID NO: 95) | METDTLLLWVLLLWVPGSTG |
| Human IgK VIII (SEQ ID NO: 96) | MEAPAQLLFLLLLWLPDTTG |
| Synthetic, modified human IgK VIII | MEAPAQLLFLLLLWLPSSRA |
| (SEQ ID NO: 97) | |
| tPA (SEQ ID NO: 98) | MDAMKRGLCCVLLLCGAVFVSPS |
| SEAP (SEQ ID NO: 99) | MLLLLLLLGLRLQLSLG |
| Synthetic consensus (SEQ ID NO: 100) | MLLLLLLLLLLALALA |
| Synthetic secrecon (SEQ ID NO: 101) | MWWRLWWLLLLLLLLWPMVWA |

### Vectors

The CAR-engager-encoding nucleic acid may be introduced to a cell by a suitable vector. In embodiments, wherein the ectodomain and the immune effector domain are linked chemically, e.g., via click chemistry, the CAR-encoding nucleic acids may be introduced into one or more cells by separate vectors. A vector is configured so as to contain the elements necessary to effect transport into the immune cell and effect expression of the nucleic acid(s) after transformation. Such elements include an origin of replication, a poly-A tail sequence, a selectable marker, and one or more suitable sites for the insertion of the nucleic acid sequences, such as a multiple cloning site (MCS), one or more suitable promoters, each promoter operatively linked to the insertion sites of the nucleic acid sequences and the selectable marker, and additional optional regulatory elements.

The term “promoter” as used herein refers to a nucleic acid sequence that regulates, directly or indirectly, the transcription of a corresponding nucleic acid coding sequence to which it is operably linked, which in the context of the present disclosure, is a CAR-engager protein. A promoter may function alone to regulate transcription, or it may act in concert with one or more other regulatory sequences (e.g., enhancers or silencers, or regulatory elements that may be present in the nucleic acid sequences or the vector). Promoters are located near the transcription start sites of genes, on the same strand and upstream on the DNA (toward the 5′ region of the sense strand). Promoters typically range from about 100-1000 base pairs in length.

The term “operatively linked” as used herein is to be understood that a nucleic acid sequence is spatially situated or disposed in the vector relative to another nucleic acid sequence, e.g., a promoter is operatively linked to drive the expression of a nucleic acid coding sequence (e.g., the CAR-engager-encoding nucleic acid sequence).

In some embodiments, a single vector contains a single promoter operatively linked to the CAR-engager-encoding nucleic acid. In some embodiments, a single vector contains a single promoter operatively linked to the ectodomain-encoding nucleic acid and the immune cell effector domain-encoding nucleic acid. In some of these embodiments, the nucleic acids are separated by a nucleic acid encoding a self-cleaving peptide or an internal ribosome entry site (IRES). In some embodiments, the single vector contains a first promoter operatively liked to the ectodomain-encoding nucleic acid and a second promoter operatively liked to the immune cell effector domain-encoding nucleic acid.

In some embodiments, two vectors are provided. In some embodiments, a first vector contains a promoter operatively linked to the ectodomain-encoding nucleic acid and a second vector contains a promoter operatively linked to the immune cell effector domain-encoding nucleic acid.

In some embodiments, the vector contains a strong mammalian promoter, for example a cytomegalovirus (CMV) promoter, a simian virus 40 (SV40) early promoter, synthetic promoters (e.g., RPBSA (synthetic, from Sleeping Beauty), or CAG (synthetic, CMV early enhancer element, chicken β-Actin, and splice acceptor of rabbit β-Globin)) or promoters derived from the β-actin, phosphoglycerate kinase (PGK), or factor EF1α genes. In some embodiments, the promoter may contain a core region located close to the nucleic acid coding sequence. In some embodiments, the promoter is modified to remove methylation sensitive motifs (e.g., a cytosine nucleotide is followed by a guanine nucleotide, or “CpG”), or by the addition of a regulatory sequence that binds transcriptional factors that repress DNA methylation. In some embodiments, the vector includes A/T-rich, nuclear matrix interacting sequences, known as scaffold matrix attachment regions (S/MAR), which enhance transformation efficiency and improve the stability of transgene expression.

In some embodiments, the vector is a viral vector, for example, a retroviral vector, a lentiviral vector, an adenoviral vector, a herpesvirus vector, an adenovirus, or an adeno-associated virus (AAV) vector. The construction of lentiviral vectors has been described, for example, in U.S. Pat. Nos. 5,665,577, 5,981,276, 6,013,516, 7,090,837, 8,119,119 and 10,954,530.

In other embodiments, the vector is a non-viral vector, representative examples of which include plasmids, mRNA, linear single stranded (ss) DNA or linear double stranded (ds) DNA, minicircles, and transposon-based vectors, such as Sleeping Beauty (SB)-based vectors and piggyBac(PB)-based vectors. In yet other embodiments, the vector may include both viral and non-viral elements.

In some embodiments the vector is a plasmid. In addition to a promoter operatively linked to the nucleic acids, the plasmid may also contain other elements e.g., that facilitate transport and expression of the nucleic acid in an immune cell. The plasmid may be linearized with restriction enzymes, in vitro transcribed to produce mRNA, and then modified with a 5′ cap and a 3′ poly-A tail. In some embodiments, the vector multiple plasmids, a first plasmid encoding a first proteinaceous entity (e.g., the ectodomain of the CAR-engager) and a second plasmid encoding a second proteinaceous entity (e.g., the immune effector domain of the CAR-engager).

### Cells

One aspect of the present disclosure is a genetically modified (or transformed) cell containing a vector that contains a nucleic acid encoding the CAR-engager or components of the CAR-engager for the purpose of making and purifying the CAR-engager protein.

Cells useful for the cloning and other manipulations of these vectors are conventional. Cells from various strains of E. coli may be used for replication of the vectors and other steps in the construction of the CAR-engagers of this disclosure.

Suitable host cells or cell lines for the expression of the nucleic acid-encoding CAR-engagers include eukaryotic cells. In some embodiments, the cells are a mammalian cell line. In some embodiments, the cells are mammalian cells such as CHO (e.g., DG44, CHO-S), fibroblast cells (e.g., 3T3, COS), embryonic cells (e.g., PER.C6, HEK (e.g., HEK.293)), somatic cell hybrids (e.g., Sp2/0), and cancer cells, for example, myeloma cells (e.g., NS0 (NS zero)). In some embodiments, the nucleic acids encoding the CAR-engager is expressed in a CHO or a myeloma cell. Human cells may be used, thus enabling the expressed CAR-engager to be modified with human glycosylation patterns. The selection of suitable mammalian cells and methods for transformation, culture, amplification, screening and product production and purification are known in the art. See, e.g., Green et al., eds., Molecular Cloning: A Laboratory Manual, 5^{th} ed., Cold Spring Harbor Laboratory Press, New York, 2012.

In some embodiments, the cells are prokaryotic. Prokaryotic (i.e., bacterial) cells may prove useful as host cells suitable for the expression of the nucleic acids encoding CAR-engagers (see, e.g., Pluckthun, Immunol. Rev. 130:151-188 (1992)). However, due to the tendency of proteins expressed in bacterial cells to be in an unfolded or improperly folded form or in a non-glycosylated form, any CAR-engagers produced in a bacterial cell would be screened for retention of function (e.g., CAR binding ability). If the CAR-engager expressed by the bacterial cell was produced in a properly folded form, that bacterial cell would be a desirable host, or in alternative embodiments the CAR-engager may express in the bacterial host and then be subsequently re-folded. For example, various strains of E. Coli used for expression are well-known as host cells in the field of biotechnology. Various strains of B. Subtilis, Streptomyces, other bacilli and the like may also be employed.

After expression in a cell, the CAR-engagers are isolated from the cell (e.g., cell lysates) or from the medium in which the cell is cultured. Protein isolation techniques are known in the art. Representative isolation techniques include chromatography, affinity chromatography, nickel-nitrilotriacetic acid (Ni-NTA) affinity chromatography, high performance liquid chromatography (HPLC), hydroxylapatite chromatography, protein A-Sepharose, gel electrophoresis, and dialysis. In some embodiments, the affinity chromatography resin is a Protein A affinity chromatography resin or a Protein G affinity chromatography resin. Additional protein isolation systems and methods are known in the art. See, e.g., U.S. Pat. Nos. 5,169,936, 6,267,958, 8,357,778, 9,630,165, 9,708,399, 10,023,608, 10,207,229, 11,369,703, and 11,390,668, U.S. Patent Application Publications 2008/0090995, 2012/0244075, 2017/0158760, 2019/0276492, and 2021/0206815, and Traunecker et al., Embo J. 10(12):3655-9 (1991).

In some embodiments, the CAR-engager is encoded by two or more nucleic acids, e.g., the ectodomain-containing moiety is encoded by one nucleic acid and the immune cell effector domain is encoded by a second nucleic acid. In these embodiments, after expression in a suitable cell and purification, the purified ectodomain and the purified immune cell effector may be connected by a suitable chemical connection reaction, described above.

### Pharmaceutical Compositions

Pharmaceutical compositions of the disclosure include effective amounts of the CAR-engager and a pharmaceutically acceptable carrier. The term “effective amount” as used herein refers to a sufficient amount of CAR-engager to provide the desired effect, e.g., the amount of a CAR-engager to bind to a CAR-expressing immune cell. The amount of CAR-engager administered to a subject will vary between wide limits, depending upon the location, type, and severity of the cancer, the age, body weight, and condition of the individual to be treated, etc. A physician will ultimately determine appropriate doses to be used. The CAR-engager in the pharmaceutical composition may be in the form of a monomer (in embodiments lacking a dimerization domain), homodimer, or heterodimer, as described herein.

The amount of CAR-engager administered to a subject may vary between wide limits, depending upon numerous factors such as the location, type, and severity of the cancer, and the age, body weight, and condition of the individual to be treated. A physician will ultimately determine appropriate amount of CAR-engager and doses to be used. Typically, the CAR-engager will be administered in a series of doses. In some embodiments, the effective amount of the CAR-engager is between approximately 50 to approximately 180 mg per subject per dose. In some embodiments, the effective amount of the CAR-engager is between approximately 1 to approximately 18 mg per kg of subject body weight.

Compositions may be provided as sterile solid or liquid preparations. Solid preparations may be reconstituted and diluted into a liquid preparation before use, e.g., with carriers containing isotonic aqueous solutions, suspensions, emulsions, dispersions, or viscous solutions, which may be buffered to a selected pH. Liquid carriers include aqueous or non-aqueous carriers alike. Representative examples of liquid carriers include sterile water for injection, saline, Lactated Ringer Injection solution, phosphate buffered saline, a soluble protein, soluble sugars (e.g., dextrose), dimethyl sulfoxide (DMSO), polyol (e.g., glycerol, propylene glycol, liquid polyethylene glycol, and the like), ethanol, and suitable mixtures thereof. In some embodiments, the liquid carrier includes a protein dissolved or dispersed therein, representative examples include serum albumin (e.g., human serum albumin, recombinant human albumin), gelatin, and casein. The compositions are typically isotonic, i.e., they have the same osmotic pressure as blood. Citric acid, sodium chloride, sugars, polyalcohols, and isotonic electrolyte solutions (e.g., Plasma-Lyte®) may be used to achieve the desired isotonicity. Depending on the carrier, other excipients may be added, e.g., wetting, dispersing, or emulsifying agents, gelling and viscosity enhancing agents, preservatives and the like as known in the art. In some embodiments, the compositions include citric acid, ethylenediaminetetraacetic acid (EDTA), and polysorbate 20 with a pH range between about 6.8 to about 7.2.

### Cancer

In some aspects, the present disclosure is directed to treating a cancer in a subject. The method entails administering to a subject in need thereof a pharmaceutical composition containing a CAR-engager described herein. The term “cancer” as used herein refers to a disease or disorder characterized by excess proliferation or reduced apoptosis in a subject. Cancers that may be treated with the CAR-engager disclosed herein include both hematopoietic cancers and cancers characterized by the presence of a solid tumor.

The term “subject” (or “patient”) as used herein includes all members of the animal kingdom prone (or disposed) to or suffering from the indicated cancer. In some embodiments, the subject is a human. Therefore, a subject “having a cancer” or “in need of” treatment according to the present disclosure broadly embraces subjects who have been positively diagnosed, including subjects having active disease who may have been previously treated with one or more rounds of therapy, and subjects who are not currently being treated (e.g., in remission) but who might still be at risk of relapse, and subjects who have not been positively diagnosed but who are predisposed to a cancer (e.g., on account of the basis of prior medical history and/or family medical history, or who otherwise present with a one or more risk factors such that a medical professional might reasonably suspect that the subject was predisposed to cancer).

The terms “treat”, “treating”, and “treatment” as used herein refer to any type of intervention, process performed on, or the administration of an active agent to the subject in need thereof with the therapeutic objective (“therapeutic effect”) of reversing, alleviating, ameliorating, inhibiting, diminishing, slowing down, arresting, stabilizing, or preventing the onset, progression, development, severity or recurrence of a symptom, complication or condition, or biochemical indicia associated with a cancer.

In some embodiments, the cancer is a hematopoietic cancer. Representative hematological cancers include plasma cell neoplasm (e.g., myeloma, multiple myeloma, relapsed or refractory multiple myeloma, plasma cell myeloma, extramedullary multiple myeloma, monoclonal gammopathy of unknown significance (MUGS), asymptomatic smoldering multiple myeloma, or solitary plasmacytoma), lymphoma (e.g., Hodgkin's lymphoma, non-Hodgkin's lymphoma, Burkitt's lymphoma, plasmablastic lymphoma, plasmacytoid lymphoma, or diffuse large B-cell lymphoma), leukemia (e.g., relapsed or refractory acute B lymphocytic leukemia, or relapsed or refractory acute lymphoblastic leukemia), and carcinomas (e.g., Waldenstrom macroglobulinemia or glioblastoma (astrocytoma)). In these embodiments, the therapeutic effect might include on or more art-recognized indicia of therapeutic efficacy, representative examples of which include prevention or prolongation of metastases, improvement in survival time, total/complete or partial remission of a cancer, e.g., no detectable cancer cells and less tumor cells or smaller tumors, respectively, or a reduction in tumor cell number. In some embodiments, the hematopoietic cancer is multiple myeloma, lymphoma, or leukemia.

In some embodiments, the cancer is characterized by the presence of a solid tumor. In some embodiments, the cancer is a bladder cancer (e.g., transitional cell carcinoma, also called urothelial carcinoma), kidney cancer (e.g., renal cell carcinoma (RCC), kidney renal clear cell carcinoma (KIRC), transitional cell cancer, or Wilms tumor), skin cancer (e.g., melanoma, skin cutaneous melanoma (SKCM), basal cell carcinoma, and squamous cell carcinoma of the skin), lung cancer (e.g., small cell lung cancer, non-small cell lung cancer, including lung adenocarcinoma (LUAD) and lung squamous cell carcinoma (LUSC)), head and neck cancer (e.g., squamous cell carcinoma of the head and neck (SCCHN) also called head and neck squamous cell carcinoma (HNSC), laryngeal and hypopharyngeal cancer, nasal cavity and paranasal sinus cancer, nasopharyngeal cancer, oral and oropharyngeal cancer, and salivary gland cancer), colon or rectal cancer (e.g., colorectal carcinoma (CRC), colon adenocarcinoma (COAD), rectum adenocarcinoma (READ)), ovarian cancer (e.g., cystadenocarcinoma, ovarian embryonal carcinoma, ovarian adenocarcinoma, epithelial ovarian carcinomas, fallopian tube cancer, and primary peritoneal cancer), endometrial cancer, cervical cancer (e.g., cervical squamous cell carcinoma and endocervical adenocarcinoma (CESC)), prostate cancer (e.g., prostate adenocarcinoma (PRAD)), and stomach cancer (e.g., stomach adenocarcinoma (STAD)).

In some embodiments, the cancer is characterized as being in a state of minimal residual disease (MRD). MRD is a state at which a cancer patient has a small number of cancer cells that remain in the body after treatment. The number of remaining cells may be so small that they do not cause any physical signs or symptoms of the cancer, and often may not be detectable through traditional methods, such as viewing cells under a microscope and/or by tracking abnormal serum proteins in the blood.

The amount of cancer antigens present in a subject in a state of MRD are limited. And this limited presence of the cancer antigen may not adequately support the proliferation and efficacy of CAR immune cells. The additional presence of a CAR-engager presents the CAR immune cells with not only additional cancer antigen, but also a supportive immune cell effector domain that may modulate the activity of the CAR immune cell to promote proliferation, efficacy, and/or persistence.

In some embodiments, the subject receiving an administration of CAR-engager is in a state of MRD. In some embodiments, the method of treating cancer involves treatment of a state of minimal residual disease (MRD) in the subject. In some embodiments, the method of treating cancer involves elimination of MRD in the subject.

To test for MRD, samples from either a blood draw or a bone marrow aspiration may be used. The most widely used tests to measure MRD are flow cytometry, polymerase chain reaction (PCR) and next-generation sequencing. Methods that may be suitable for use in measuring MRD are described in, e.g., U.S. Pat. Nos. 8,124,353, 9,528,160, 10,280,462, 11,618,787, and 11,633,426, and U.S. Patent Application Publications 2011/0294148, and 2022/0380852.

### Administration

In some embodiments, the methods of the present disclosure entail administration of an effective amount of the CAR-engager to a cancer patient who had received a prior administration of immune cells containing a CAR that contains an extracellular domain that binds the ectodomain of the CAR-engager, a transmembrane domain, and an intracellular domain comprising a stimulatory domain. In some embodiments, the CAR-engager is administered to the subject subsequent to a determination that the CAR-immune cells have lost vitality or persistence in the subject. This determination may be made in accordance with known techniques. In some embodiments, for example, a sample is obtained from the subject after the administration of the immune cells. The concentration of immune cells present within the sample may be used to calculate the difference between the concentration of the immune cells administered to the subject and the concentration of the immune cell measured in the sample. The CAR-engager may be administered once the measured immune cell concentration is less than the administered immune cell concentration. In some embodiments, the CAR-engager is administered once the measured immune cell concentration is less than 90%, less than 80%, less than 70%, less than 60%, less than 50%, less than 25%, less than 10%, or less than 5% of the administered concentration of immune cells.

In some embodiments, administration of the CAR-engager is conducted at least about 1 week, at least about 2 weeks, at least about 1 month, at least about 2 months, at least, at least about 3 months, at least about 6 months, at least about 9 months, or at least about a year after administering the CAR-immune cells.

In some embodiments, the CAR-engager is administered once every 3 weeks (a 21-day cycle) as an infusion over about 30 to about 90 minutes. In some embodiments, the CAR-engager is administered for 5 consecutive days every 21 days and repeated for 8 cycles.

In other embodiments, the method further entails co-administering an effective number of immune cells expressing a CAR (also referred to as CAR immune cells) in the same course of treatment with the administration of the CAR-engager. The term “effective number of CAR immune cells” (which indirectly includes a corresponding amount of a CAR) as used herein refers to a sufficient number of the CAR immune cells to provide the desired effect.

More broadly, the order in which the CAR-engager and CAR immune cells are administered during the same course of treatment may not be critical, provided that they are able to interact in vivo and cause the desired effect. In some embodiments, the CAR-engager is co-administered substantially simultaneously to the subject with the CAR immune cells. In some embodiments, the CAR-engager is contacted with the CAR immune cells in vitro before co-administration to the subject. In some embodiments, the CAR-engager is administered to the subject subsequent to the administration of the CAR immune cells. In some embodiments, the CAR-engager is administered to the subject prior to the administration of the CAR immune cells.

The extracellular domain of the CAR that binds the ectodomain of a cancer antigen may contain an antibody fragment. In some embodiments, the CAR binds BCMA. CAR extracellular domains that bind to BCMA are known in the art. See, e.g., FDA-approved CAR-expressing immune cells ciltacabtagene autoleucel (Carvykti®), and idecabtagene vicleucel (Abecma®), U.S. Pat. Nos. 10,072,088, 10,683,369, 11,084,880, and 10,174,095, and U.S. Patent Application Publications 2016/0131655, 2017/0226216, 2018/0133296, 2019/0151365, 2019/0359727, 2019/0381171, 2020/0339699, 2020/0360431, 2020/0055948, and 2022/0064316. In some embodiments, the CAR extracellular domain is derived from a commercially available anti-BCMA antibody, BCMA-binding fragment, or derivative thereof, e.g., belantamab (Blenrep®), linvoseltamab (REGN5458), pacanalotamab (AMG 420), pavurutamab (AMG 701), and teclistamab (Tecvayli®). In some embodiments, the extracellular domain of the CAR will bind the BCMA ectodomain of the CAR-engager that has the amino acid sequence of SEQ ID NO: 1 or SEQ ID NO: 2.

In some embodiments, the CAR binds CD19. CAR extracellular domains that bind to CD19 are known in the art. See, e.g., FDA-approved CAR-expressing immune cells lisocabtagene maraleucel (Breyanzi®), tisagenlecleucel (Kymriah®), brexucabtagene autoleucel (Tecartus®), and axicabtagene ciloleucel (Yescarta®), U.S. Pat. Nos. 9,629,877, 10,273,300, and 10,533,055, and U.S. Patent Application Publications 2020/0392248, and 2021/0238253. In some embodiments, the CAR extracellular domain is derived from a commercially available anti-CD19 antibody, anti-CD19-binding fragment, or derivative thereof, e.g., loncastuximab (Zynlonta®), tafasitamab (Monjuvi®), denintuzumab (SGN-CD19A), and inebilizumab (Uplizna®). In some embodiments, the extracellular domain of the CAR will bind the CD19 ectodomain of the CAR-engager having the amino acid sequence of any one of SEQ ID NOs: 3-5, or 103.

In some embodiments, the CAR binds CD20. CAR extracellular domains that bind to CD20 are known in the art. See, e.g., U.S. Pat. Nos. 10,189,903, 10,442,867, 10,934,363, 11,066,457, 11,160,833, and 11,439,665, and U.S. Patent Application Publication 2018/0187149. In some embodiments, the CAR extracellular domain is derived from a commercially available anti-CD20 antibody, anti-CD20-binding fragment, or derivatives thereof, e.g., ofatumumab (Arzerra®, Kesimpta®), veltuzumab (IMMU-106), tositumomab (Bexxar®), and rituximab (Rituxan®, Riabni®, Truximab®). In some embodiments, the extracellular domain of the CAR will bind the CD20 ectodomain of the CAR-engager having the amino acid sequence SEQ ID NO: 6.

In some embodiments, the CAR binds CD22. In these embodiments, the extracellular domain of the CAR will bind a CD22 ectodomain of the CAR-engager. CAR extracellular domains that bind CD22 are known in the art. See, e.g., U.S. Pat. Nos. 9,139,649, 9,181,343, and 10,494,435, U.S. Patent Application Publications 2015/0175711, 2018/0086843, 2021/0047402, 2021/0095022, 2022/0220198, and 2022/0273710, and Fry et al., Nat. Med. 24(1):20-28 (2018). In some embodiments, the CAR extracellular domain is derived from a commercially available anti-CD22 antibody, anti-CD22-binding fragment, or derivatives thereof, e.g., bectumomab, epratuzumab, inotuzumab, moxetumomab, and epratuzumab. In some embodiments, the CAR-engager contains a CD22 ectodomain that has the amino acid sequence SEQ ID NO: 7. In some embodiments, the CAR-engager contains a CD22 ectodomain that has any one of the amino acid sequences SEQ ID NOs: 104-107.

In some embodiments, the CAR binds SLAMF7. CAR extracellular domains that bind SLAMF7 are known in the art. See, e.g., U.S. Pat. No. 10,799,536, and U.S. Patent Application Publications 2020/0024342, 2020/0283534, 2021/0230548, and 2021/0253729. In some embodiments, the CAR extracellular domain is derived from a commercially available anti-SLAMF7 antibody, anti-SLAMF7-binding fragment, or derivative thereof, e.g., elotuzumab (Empliciti®). In some embodiments, the extracellular domain of the CAR will bind the SLAMF7 ectodomain of the CAR-engager having the amino acid sequence SEQ ID NO: 10.

In some embodiments, the CAR binds PD-1. CAR extracellular domains that bind to PD-1 are known in the art. See, e.g., U.S. Pat. Nos. 10,124,023 and 11,136,392, and U.S. Patent Application Publications 2021/0061877, 2020/0281974, and 2022/0064595. In some embodiments, the CAR extracellular domain is derived from a commercially available anti-PD-1 antibody, anti-PD-1-binding fragment, or derivative thereof, e.g., balstilimab, budigalimab, cadonilimab, cemiplimab (Libtayo®), cetrelimab, dostarlimab (Jemperli®), izuralimab, nivolumab (Opdivo@), pacmilimab, pembrolizumab (Keytruda®), penpulimab, peresolimab, pidilizumab, retifanlimab, rosnilimab, sintilimab, spartalizumab, tislelizumab, toripalimab, volrustomig, vudalimab, zeluvalimab, and zimberelimab. Therefore, in some embodiments, the extracellular domain of the CAR will bind the PD-1 ectodomain of the CAR-engager having the amino acid sequence SEQ ID NO: 11.

In some embodiments, the CAR binds receptor tyrosine kinase KIT proto-oncogene, (KIT). CAR extracellular domains that bind to KIT are known in the art. See, e.g., U.S. Patent Application Publications 2017/0335281, 2020/0048359, 2020/0071397, and 2021/0299177. In some embodiments, the CAR extracellular domain is derived from a commercially available anti-KIT antibody, anti-KIT-binding fragment, or derivative thereof, e.g., barzolvolimab. In some embodiments, the extracellular domain of the CAR will bind the KIT ectodomain of the CAR-engager having the amino acid sequence SEQ ID NO: 12.

In some embodiments, the CAR binds CD38. CAR extracellular domains that bind to CD38 are known in the art. See, e.g., U.S. Pat. Nos. 10,709,775, 10,799,536, 10,836,998, and 11,365,394 and U.S. Patent Application Publications 2017/0296623, 2019/0135894, 2019/0135937, 2020/0308541, 2021/0046118, and 2022/0202859 In some embodiments, the CAR extracellular domain is derived from a commercially available anti-CD38 antibody, anti-CD38-binding fragment, or derivative thereof, e.g., daratumumab (Darzalex®), isatuximab (Sarclisa®), and mezagitamab. In some embodiments, the extracellular domain of the CAR will bind the CD38 ectodomain of the CAR-engager having the amino acid sequence SEQ ID NO: 14.

The intracellular domain of the CAR contains a signaling domain that enables intracellular signaling and immune cell function. The signaling domain may include a primary signaling domain and/or a co-stimulatory signaling domain. In some embodiments, the intracellular domain is capable of delivering a signal approximating that of natural ligation of an ITAM-containing molecule or receptor complex such as a TCR receptor complex.

In some embodiments, the signaling domain includes a plurality, e.g., 2 or 3, costimulatory signaling domains, e.g., selected from 4-1BB, CD3ζ, CD28, CD27, ICOS, and OX40. In some embodiments, the signaling domain may include a CD3ζ domain as a primary signaling domain, and any of the following pairs of co-stimulatory signaling domains from the extracellular to the intracellular direction: 4-1BB-CD27; CD27-4-1BB; 4-1BB-CD28; CD28-4-1BB; OX40-CD28; CD28-OX40; 4-1BB-CD3ζ; CD3ζ-4-1BB; CD28-CD3ζ; CD3ζ-CD28; CD28-4-1BB and 4-1BB-CD28. In some embodiments the primary signaling domain is derived from CD3ζ, CD27, CD28, CD40, KIR2DS2, MyD88, or OX40. In some embodiments, the co-stimulatory signaling domain is derived from one or more of CD3γ, CD3δ, CD3ε, CD3ζ, CD4, CD5, CD8α, CD9, CD16, CD22, CD27, CD28, CD33, CD37, CD40, CD45, CD68, CD72, CD80, CD86, CD137 (4-1BB; TNFRSF9), CD154, CLEC-1, 4-1BB, DAP10 (hematopoietic cell signal transducer ((HCST)), DAP12 (TYROBP), Dectin-1, FcαRI, FcγRI, FcγRII, FcγRIII, IL-2RB, ICOS, KIR2DS2, MyD88, OX40, and ZAP70.

A representative CAR with a CD3ζ stimulatory signaling domain is the FDA-approved CAR-expressing immune cells tisagenlecleucel (Kymriah®). Representative CARs with CD3ζ and 4-1BB co-stimulatory signaling domains are the FDA-approved CAR-expressing immune cells idecabtagene vicleucel (Abecma®), lisocabtagene maraleucel (Breyanzi®), and ciltacabtagene autoleucel (Carvykti®). Representative CARs with CD28 and CD3ζ co-stimulatory signaling domains are the FDA-approved CAR-expressing immune cells brexucabtagene autoleucel (Tecartus®) and axicabtagene ciloleucel (Yescarta®).

In some embodiments, the CAR immune cell is a T cell. In some embodiments, the CAR immune cell is a NK cell. Additional CAR immune cells are known in the art, e.g., U.S. Pat. Nos. 5,906,936, 7,446,190, 7,741,465, 8,389,282, 8,399,645, 9,422,351, 9,790,267, 9,885,298, 10,124,023, 10,815,301, and 11,433,100, and U.S. Patent Application Publications 2019/0375815, 2020/0281973, 2021/0300986, 2022/0056101, and 2022/0193138.

Without being bound by theory, the interaction between CAR-engager and CAR does not cause a clustering effect and synapse formation. In contrast, cancer antigens cause clustering effects and synapse formation between CARs and cancer antigens on the CAR immune cell surface. Therefore, the binding of the CAR-engager to a CAR immune cell is short-term and reversible, which ensures that a CAR immune cell may bind to a cancer cell through the CAR in the presence of CAR-engagers.

The number of CAR immune cells administered to a subject will vary between wide limits, depending upon the location, type, and severity of the cancer, the age, body weight, and condition of the individual to be treated, etc. A physician will ultimately determine appropriate number of cells and doses to be used. Typically, the CAR immune cells will be given in a single dose.

In some embodiments, the effective number of the CAR immune cells is between approximately 1×10⁵ to approximately 1×10¹⁰ cells per subject. In some embodiments, the effective number of the CAR immune cells is between approximately 1×10⁵ to approximately 6×10⁸ cells per kg of subject body weight.

Since the CAR-engager augments CAR immune cell functionality and persistence in vivo, it may reduce the cellular dose needed for CAR immune cell therapy, which in turn may reduce adverse side effects (e.g., cytokine release syndrome) caused by the larger doses typically used in the clinic. Therefore, in some embodiments, the effective number of the CAR immune cells is between approximately 1×10⁴ to approximately 1×10⁷ cells per subject. In some embodiments, the effective number of the CAR immune cells is between approximately 1×10⁴ to approximately 6×10⁵ cells per kg of subject body weight.

Compositions containing an effective amount of the CAR-engager and an effective number of the CAR immune cells may be administered to a subject for the treatment of a cancer by any medically acceptable route. The CAR-engagers and CAR immune cells are typically delivered intravenously, although they may also be introduced into other convenient sites (e.g., to an affected organ or tissue) or modes, as determined by an attending physician.

Expansion and differentiation agents can be provided prior to, during or after administration of the cells to increase differentiation, expansion, and/or persistence of the CAR immune cells (e.g., T cells and NK cells).

Administration of the CAR immune cells may be autologous or allogeneic. For example, immune cells or progenitors thereof can be isolated from a tissue of body fluid from one subject prior to administration to the same subject (autologous) or a different, compatible subject (allogeneic).

In some embodiments, the CAR-engager is administered cyclically, for example, administered once a week, once every two weeks, once every three weeks. The cycle is repeated, for example, for 2 cycles, 3 cycles, 5 cycles, 8 cycles. In some embodiments, the CAR-engager is administered for a period of consecutive days before cyclic administration, for example, administered once a day for five days and once every three weeks thereafter. In some embodiments, the CAR-engager is administered as an intravenous infusion over a period of time. Representative infusion times are 30 minutes, 60 minutes, and 90 minutes. In some embodiments, the infusion time is between 30 and 60 minutes. In some embodiments, the first administration is infused into a patient for 90 minutes and administrations are infused into a patient for 30 minutes.

### Combination Therapy

In some embodiments, the present methods may include co-administration of another anti-cancer agent with the CAR-engager or the CAR-engager and the CAR immune cells. The term “co-administered” includes substantially contemporaneous administration, by the same or separate dosage forms, or sequentially, e.g., as part of the same treatment regimen or by way of successive treatment regimens. Thus, if given sequentially, at the onset of administration of the second therapy, the first of the two therapies is, in some cases, still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents exactly at the same time.

Anti-cancer agents that may be used in combination with the inventive cells are known in the art. See, e.g., U.S. Pat. No. 9,101,622 (Section 5.2 thereof). An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of cancerous cells. This process may involve contacting the cancer cells with recipient cells and the agent(s) or multiple factor(s) at the same time. This may be achieved by contacting the cancer cells with a single composition or pharmacological formulation that includes both agents, or by contacting the cancer cells with two distinct compositions or formulations, at the same time, wherein one composition includes recipient cells and the other includes the second agent(s).

In some embodiments, CAR-engagers and the CAR immune cells of the present disclosure are used in conjunction with or following prior therapies such as chemotherapeutic, radiotherapeutic, immunotherapeutic intervention, targeted therapy, pro-apoptotic therapy, or cell cycle regulation therapy.

In some embodiments, the CAR-engager and CAR immune cells of the present disclosure are used in conjunction with high-dose chemotherapy prior to the administering of the genetically modified immune cells. In some embodiments, bone marrow cells or peripheral blood stem cells are administered subsequent to the high-dose chemotherapy.

In some embodiments, the CAR-engager and CAR immune cells of the present disclosure are used in conjunction with an effective amount of thalidomide, lenalidomide, bortezomib, or a combination thereof.

Additional potentiating treatments that may be used in conjunction with the genetically modified immune cells of the present disclosure include melphalan. Melphalan (Alkeran®, Evomela®), an alkylating antineoplastic agent, is used for high-dose conditioning prior to hematopoietic stem cell transplant in patients with multiple myeloma, as well as for palliative treatment of multiple myeloma and for the palliation of non-resectable epithelial carcinoma of the ovary. Melphalan is also used to treat AL amyloidosis, neuroblastoma, rhabdomyosarcoma, breast cancer, ocular retinoblastoma, some conditioning regiments before bone marrow transplant, and in some cases, malignant melanoma. Melphalan may be administered in pill form by mouth. Typically, in 2 mg doses taken on an empty stomach. In some cases, Melphalan may be administered as an injection or intravenous infusion. Dosing depends on weight, height, disease and disease state, and the subject's general health.

### Immunotherapy

Immunotherapy, including immune checkpoint inhibitors may be employed to treat a diagnosed cancer. Immune checkpoint molecules include, for example, PD-1, PDL1, CTLA4, KIR, TIGIT, TIM-3, LAG-3, BTLA, VISTA, CD47, and NKG2A. Clinically available examples of immune checkpoint inhibitors include durvalumab (Imfinzi®), atezolizumab (Tecentriq®), and avelumab (Bavencio®). Clinically available examples of PD-1 inhibitors include nivolumab (Opdivo®), pembrolizumab (Keytruda®), and cemiplimab (Libtayo®). Additional inhibitors that may be useful in the practice of the present disclosure are known in the art. See, e.g., U.S. Patent Application Publications 2012/0321637, 2014/0194442, and 2020/0155520.

### Chemotherapy

Anti-cancer therapies also include a variety of combination therapies with both chemical and radiation-based treatments. Combination chemotherapies include, for example, Abraxane®, altretamine, docetaxel, Herceptin®, methotrexate, Novantrone®, Zoladex®, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, Taxol®, gemcitabien, Navelbine®, farnesyl-protein tansferase inhibitors, transplatinum, 5-fluorouracil, vincristine, vinblastine and methotrexate, or any analog or derivative variant of the foregoing and also combinations thereof.

### Radiotherapy

Anti-cancer therapies also include radiation-based, DNA-damaging treatments. Combination radiotherapies include what are commonly known as gamma-rays, X-rays, and/or the directed delivery of radioisotopes to tumor cells which cause a broad range of damage on DNA, on the replication and repair of DNA, and on the assembly and maintenance of chromosomes. Dosage ranges for radioisotopes vary widely, and depend on the half-life of the isotope, the strength and type of radiation emitted, and the uptake by the neoplastic cells and will be determined by the attending physician.

Radiotherapy may include external or internal radiation therapy. External radiation therapy involves a radiation source outside the subject's body and sending the radiation toward the area of the cancer within the body. Internal radiation therapy uses a radioactive substance sealed in needles, seeds, wires, or catheters that are placed directly into or near the cancer.

These and other aspects of the present application will be further appreciated upon consideration of the following Examples, which are intended to illustrate certain embodiments of the application but are not intended to limit its scope, as defined by the claims.

### EXAMPLES

### Example 1: Materials and Methods

The cloning and expression of proteins were done following standard approaches. Other procedures, including flow cytometric analyses, BLI imaging, CAR T cells production, cell culture and animal handling were performed following standard protocols as briefly explained below.

Generation of CAR-engagers. All genes were codon-optimized for mammalian expression in HEK293 cells, synthesized, and inserted into a vector expression system with a signal sequence for protein secretion into the supernatant. To facilitate production of the products, stable HEK293 cell lines were generated. Accordingly, HEK293 cells were transfected with pPAX2, pVSVG (packaging vectors), and the lentivirus plasmid containing the sequence of interest. The lentivirus was harvested at 48, 72, and 96 hours (h) post transfection, sedimented at 20,000×g for 2 h, and resuspended in optiMEM media. A new batch of HEK293 cells were then subjected to three rounds of transduction with the virus. Cells were allowed to recover in DMEM complete media and were subjected to puromycin selection to retain only cells that integrated the lentivirus plasmid. Cells were then expanded in four 15 cm culture dishes until they reached confluency, washed carefully with PBS, and incubated in serum-free DMEM for 24 to 48 h. The supernatant was harvested, and protein expression was confirmed via SDS-PAGE and immunoblotting. Proteins were purified by adsorption onto a nickel nitriloacetic acid (Ni-NTA) metal affinity column. Non-specifically bound proteins were removed by washing with 40 mM imidazole. The imidazole concentration was increased to 250 mM, allowing recovery of the protein of interest. The protein was further purified via size-exclusion chromatography and were stored in 50 mM HEPES buffer, pH 7.5 at −80° C. until use.

Some CAR-engagers were isolated by passage through an affinity chromatography resin, typically in the presence of a neutral phosphate buffer. The affinity chromatography resin was then subjected to an acidic buffer with a pH of about 3 to about 4, thereby washing CAR-engager off of the affinity chromatography resin. A basic buffer may be used to neutralize the acidic buffer, then a tangential flow filtration of the neutralized buffer can be performed with a formulation buffer, to isolate a concentrated and purified solution containing the CAR-engager.

Production of CART cells. The CAR construct that binds human CD19 contains an scFv derived from the anti-human CD19 antibody clone FMC69, followed by human CD28 and CD3ζ intracellular signaling domains. The CAR construct that binds human BCMA contains an scFv derived from the anti-human BCMA antibody clone MSK54, followed by human 41BB and CD3ζ intracellular signaling domains. The human signaling CAR constructs were transduced into HeLa cells that stably produce gamma-retrovirus pseudotyped with the envelope of the feline endogenous virus (RD 114), which has been shown to transduce human hematopoietic cells (HSC) with high efficiency (Ward et al., Mol. Ther. 8(5):804-12 (2003)). High viral titer clones were isolated by limiting dilution. The high expression clone was seeded and grown in DMEM complete media containing 10% FBS until 80% confluency. Media was exchanged with RPMI complete media containing 10% FBS. After 24 hours, the virus-containing media was harvested, sterile-filtered using a 0.45 μm PES filter, and utilized for producing CAR T cells.

The production of CAR T cells was adapted from previous studies. See, for example, Li et al., Methods Mol. Biol. 1514:111-118 (2017). In brief, whole blood was obtained from apheresis leukoreduction collars of platelet healthy donors, due to a high number of viable white blood cells. The whole blood was subjected to centrifugation through a Ficoll gradient to isolate PBMCs. Whole PBMCs were utilized without selecting for CD8⁺ T cells. PBMCs were resuspended in RPMI media containing 10% Fetal Bovine Serum (FBS), 200 IU/mL IL-2, 60 ng/mL IL-7, 10 ng/mL IL-15, 2 μg/mL anti-human CD3 (OKT3 clone), and 0.5 μg/mL anti-human CD28 (CD28.1 clone) at a cell concentration of 4×10⁶ cells/mL in 3 mL of media per well in a 6-well plate. After 24 hours, cells are harvested, spun down, and resuspended in the same volume of fresh media with FBS, IL-2, IL-15, and IL-7 in addition to media harvested from anti-human BCMA CAR gamma-retrovirus producing cells, resulting in PBMC inoculation with the gamma-retrovirus. The PBMCs were then plated at 4×10⁶ cells/mL in 3 mL into 6-well plates coated with 20 μg retronectin (coated with 1 mL of 20 g/mL retronectin in PBS for 24 hours at 4° C.). The PBMC underwent spinoculation in a centrifuge for 1 h at 2000×g at 30° C. and cultured at 37° C. The transduction step was repeated with fresh gamma-retrovirus containing media, cytokines, and spinoculation. Flow cytometry analysis was utilized to assess the transduction efficiency of the CAR transgene using the dsRed reporter gene and recombinant BCMA labeled with AlexaFlour-647.

In vivo experiments. For all experiments NOD/SCID/Gamma (NSG; NOD.Cg-Prkdc^{scid} IR2rg^{tm1Wjl}/SzJ) mice were used due to their immunocompromised status and ability to effectively engraft human cancer cell lines. Cells from the human multiple myeloma OPM2 cell line were used to establish a multiple myeloma mouse model in NSG mice. In vivo experiments were initiated by tail vein intravenous injection of 1×10⁶ OPM2 cells expressing GFP and Firefly Luciferase followed by biweekly Bioluminescent Imaging (BLI). Upon effective engraftment after 3 weeks, mice were intravenously injected through the tail vein with CAR T cells. BLI was performed biweekly afterwards to assess tumor burden. Quantification was measured using photons/see using Aura software.

Organ analysis. At the conclusion of an experiment, the surviving mice were sacrificed and spleen, bone marrow, blood, liver, kidney, and lungs were harvested and weighed. The liver, kidney, and lungs were minced, digested with collagenase (final concentration of 1 μg/mL collagenase), and incubated at 37° C. for 1 h. Spleens were crushed, and bone marrow was aspirated using a 30-gauge insulin needle. All processed cells were pushed through a 70 μm strainer to produce a single cell suspension of cells. Cells were resuspended in 1 mL of ammonium-chloride-potassium (ACK) lysis buffer to deplete the sample of red blood cells for 2 m at room temperature. The resulting single cell suspensions were washed with fluorescence-activated single cell sorting (FACS) buffer of PBS and 0.5% BSA, stained, and analyzed using flow cytometry.

Cell lines and culture. The OPM2 cell line, which endogenously express BCMA, were engineered to express green fluorescent protein (GFP) and Firefly Luciferase. Peripheral Blood Mononuclear Cells (PBMC) were obtained by Ficoll gradient of apheresis leukoreduction collars of platelet healthy donors. HEK293 T cells were cultured in complete DMEM (Gibco), 1% L-glutamine (Gibco), 1% non-essential amino acids (NEAA) (Gibco), 1% pyruvate (Gibco), and 1% penicillin and streptomycin (Cytiva), and 10% fetal bovine serum (FBS). OPM2, and PBMCs were cultured in complete RPMI-1640 (Gibco), 1% L-glutamine (Gibco), 1% non-essential amino acids (NEAA) (Gibco), 1% pyruvate (Gibco), and 1% penicillin and streptomycin (Cytiva), and 10% fetal bovine serum (FBS). All cells were grown in 5% CO₂, 95% air-humidified incubators at 37° C.

Mouse studies. All experiments adhered to the pertinent ethical and safety protocols. The studies were carried out under the oversight of the Dana-Farber Cancer Institute Institutional Animal Care and Use Committee (protocol no. 20-006). The xenograft models utilized herein are described in Smith et al., Mol. Ther. 26(6):1447-1456 (2018). Briefly, 8- to 12-week-old NOD-scid IL2Rγ^{null} (NSG) and NOD-scid H2-Kl^{null} H2-Ab1^{null} H2-D1^{null} IL2Rg^{null} (NSG-MHC I/II double knockout (DKO)) were either purchased from Jackson Laboratory or bred in-house. All mice were sex and age matched into groups. Xenograft models were established by intravenous injection of 1×10⁶ cells of OPM2 or Nalm6 expressing GFP and Luciferase in 200 mL of PBS. Mice received indicated treatments in 300 mL of PBS through intraperitoneal injections. Tumor burden was assessed using the IVIS® Lumina Series III (Perkin Elmer) after intraperitoneal injection of D-Luciferin (150 mg/kg, from a 15 mg/ml solution) at the indicated time. Each mouse was imaged in groups of up to five mice in the supine position at the same time points (5 min). BLI intensity was analyzed by Aura imaging analysis software (Spectral Instruments Imaging). Peripheral blood from mice was obtained by submandibular bleeding in an EDTA-coated tube and analyzed for CAR T cell detection and expansion. In brief, volume of the blood was determined in order to calculate absolute values. Samples were then centrifuged, and serum harvested. Cell pellets were resuspended in 500-1000 mL of ACK Lysis Buffer (150 mM NH₄Cl, 10 mM KHCO₃, 0.1 mM EDTA) for 1 minute. Cells were then washed twice in FACS buffer, PBS+1% Bovine Serum Albumin (BSA). Samples were then stained with anti-CD45-PacificBlue (1:50, Biolegend), anti-CD4-PE/Dazzle594 (1:50, Biolegend), anti-CD8-FITC (1:50, Biolegend), anti-CCR7-AlexaFluor700 (1:50, Biolegend), anti-CD62L-PE (1:50, Biolegend), anti-CD45RO-PerCP/Cy5.5 (1:50, Biolegend), anti-CD45RA-APC/Fire750 (1:50, Biolegend), anti-PD1-BV605 (1:50, Biolegend), anti-HLA-DR-PE/Cy7 (1:50, Biolegend), anti-CD69-BV421 (1:50, Biolegend), and recombinant BCMA-AlexaFlour647 (made in-house). Samples were processed on a Sony SP6800 Spectral Analyzer. Flow rate and acquisition time was noted to calculate absolute values. All experiments were performed in a blinded and randomized fashion. Animals were euthanized at the end of the experiment or when they met prespecified endpoints according to the IACUC protocols. Upon endpoint, major immune organs (spleen and bone marrow) as well as essential organs where possible metastatic lesions can form (liver, lung, and kidney) were harvested, weighed, and analyzed. In brief, spleen was crushed using a plunger and passed through a 40 m strainer to acquire a single cell suspension. Bone marrow was aspirated using a 30-gauge insulin needle. Liver, lung, and kidney were diced using surgical scissors in 3 mL of Digestion buffer (1 mL RPMI+2 mL PBS). Collagenase, Type 1 (Worthington) was added at a final concentration of 100 mg/mL and incubated at 37° C. for 1 hour. The resulting samples were passed through a 40 μm strainer to acquire a single-cell suspension. Samples were then stained with same antibodies used to stain blood samples and analyzed using a Sony SP6800 Spectral Analyzer. Flow rate and acquisition time were noted to calculate absolute values.

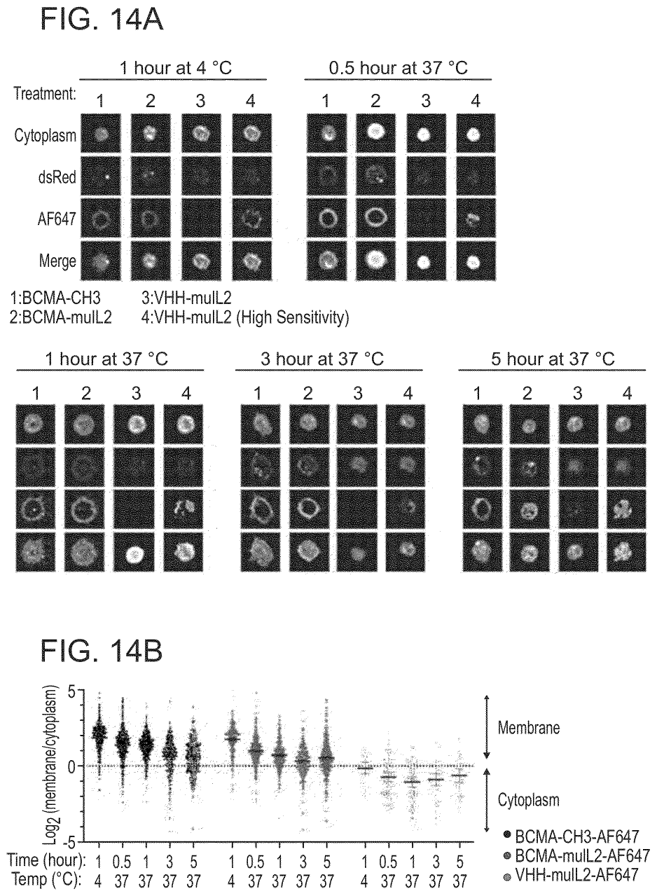

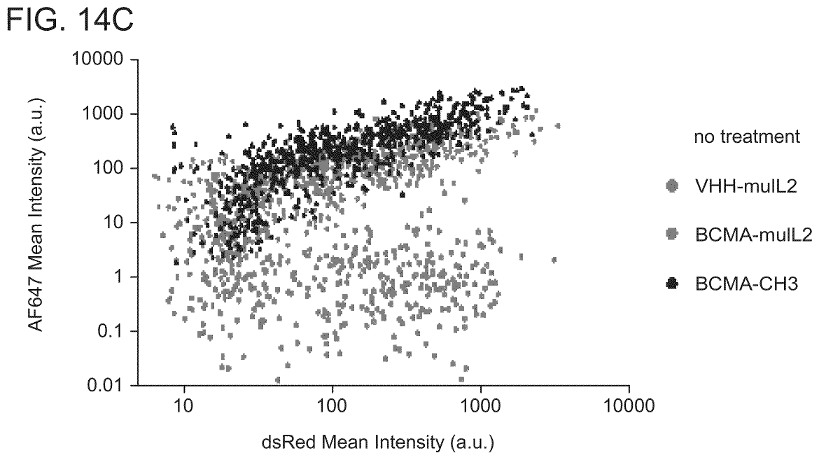

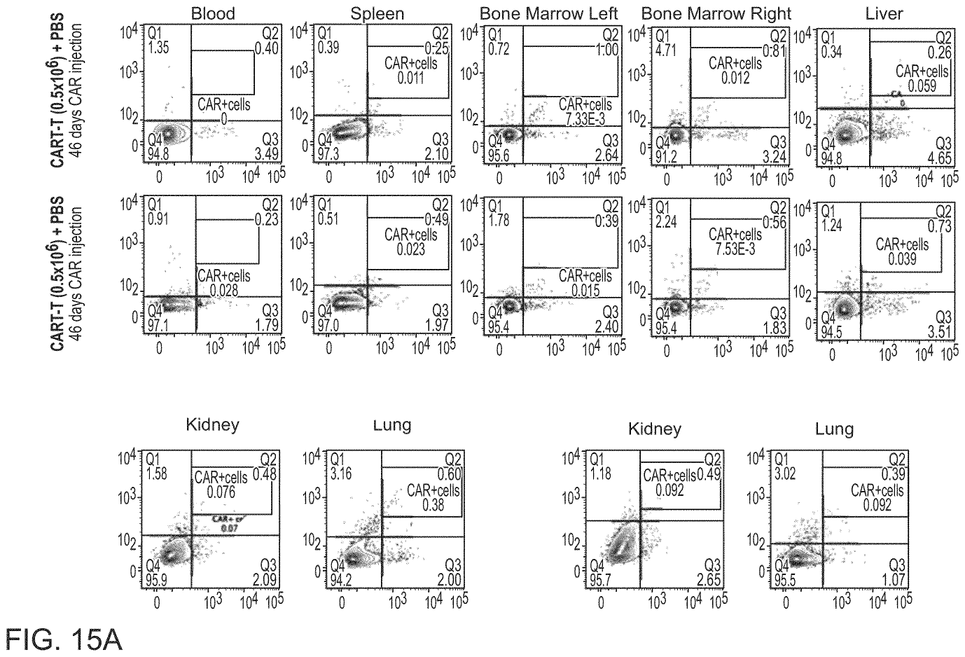

Microscopy. Cells were first stained with CellTracker Blue CMAC and seeded on poly-d-lysine-coated coverslips. Subsequently, the samples were incubated with the indicated treatment, labeled with Alexa647, at the specified time and temperature. After fixation using BD Cytofix buffer, the cells were imaged using the Leica THUNDER Imager. The intensity cut-off for the AlexaFluor647 channel was set at 2000 for all images, except for the VHH-muIL2 samples shown on the right (condition 4), for which, the image sensitivity was enhanced by 25-fold (intensity cut-off 80) to visualize the Alexa647 signal. Quantitative analysis of the fluorescence intensity was analyzed by aligning the base 2 logarithm of the ratio of integrated intensity of the membrane to the cytoplasm of the cells (see y-axis of FIG. 14B). For BCMA-muIL2 and BCMA-CH3 (conditions 1 and 2), only cells with a mean intensity to background ratio above 4 based on the dsRed channel were analyzed as they were identified as CAR⁺ T cells. For VHH-muIL2, cells with a mean intensity to background ratio greater than 2 based on the Alexa647 channel were selected to eliminate the background artifacts. Image quantification was performed using ImageJ software.

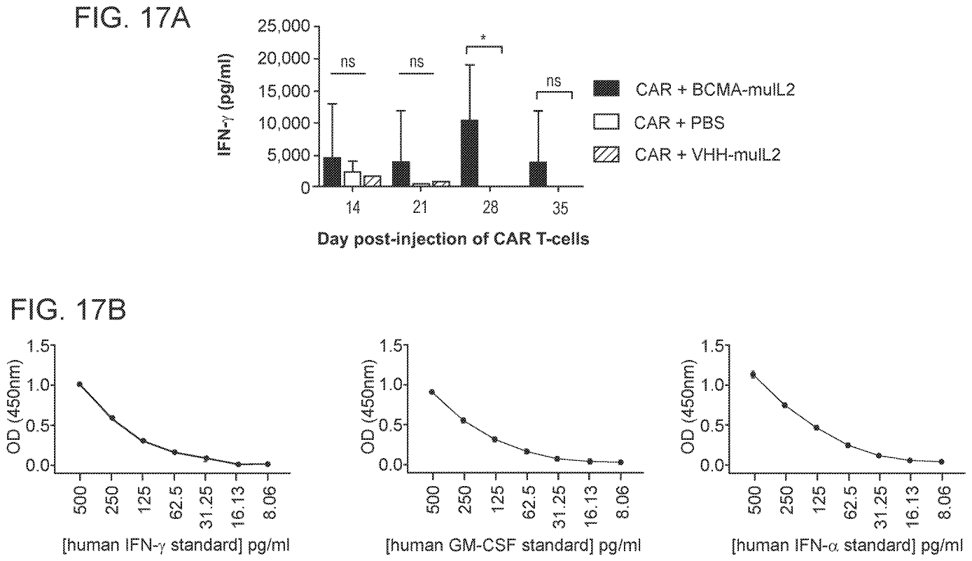

ELISA. ELISA analyses were performed to measure the levels of human T cell-derived cytokines in the serum of mice that received OPM2 cancer cells followed by a low dose of CAR T cells, as shown in FIG. 17A. The cytokines analyzed with the ELISA MAX™ Standard Set (Biolegend) included IFN-γ, GM-CSF, and TNF-α following the manufacturer's provided protein; however, only IFN-γ was detectable in the collected samples. Serum samples were diluted at a ratio of 1:40. The same plates were used to incubate both the standard samples and the serum samples, and a standard curve was plotted for each cytokine.

### Example 2: BCMA-Containing CAR-Engager In Vitro Characterization

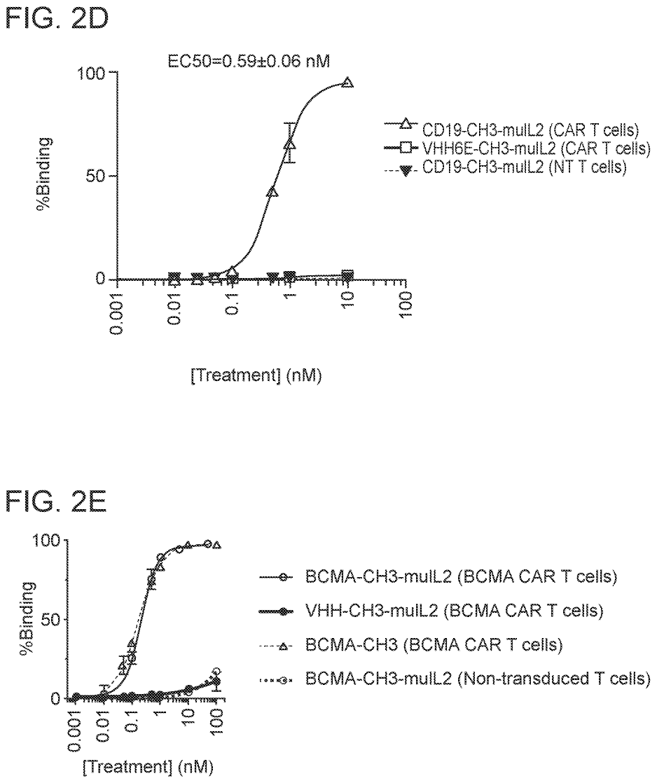

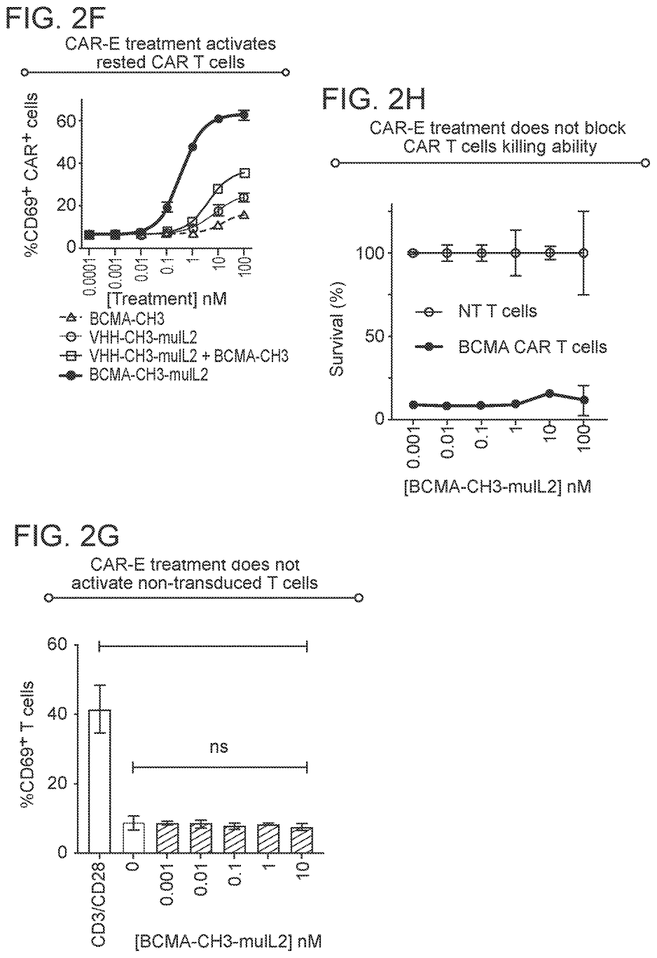

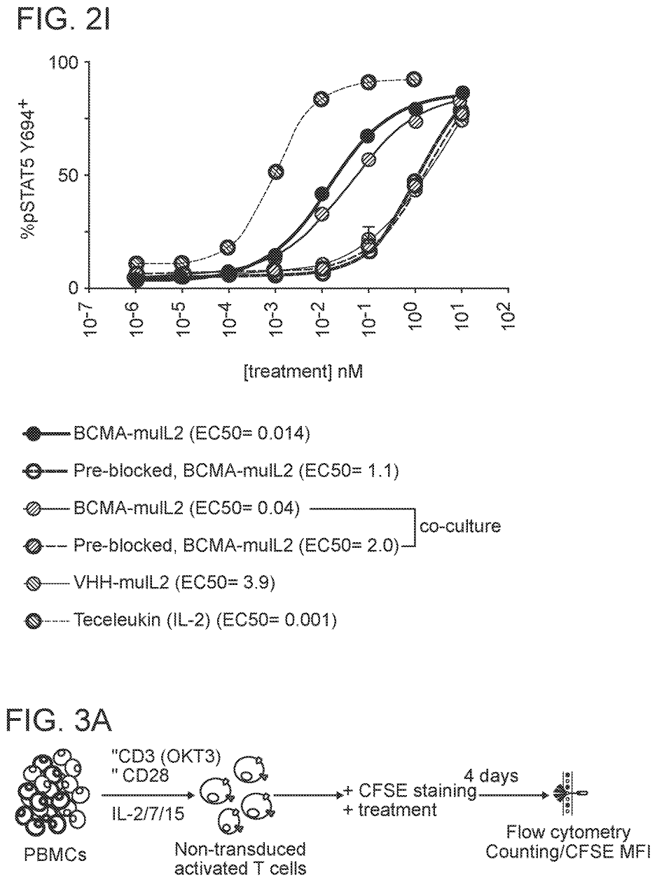

A Fusion protein consisting of the human BCMA ectodomain was fused with a two low-affinity mutated human IL-2 (muIL2) domains, and to improve pharmacokinetics and enhance stability, the CH3 domain (Feige et al., Trends Biochem. Sci. 35(4):189-198 (2010)) of human IgG1 (approximately 14 kDa in size) was incorporated between the antigen and the muIL2 (FIG. 2B) (Quayle et al., Clin. Cancer Res. 26(8):1953-1964 (2020)). Consequently, the BCMA-muIL2 CAR-engager preferentially delivers the low-affinity IL-2 to the surface of CAR T cells through antigen-to-CAR specific binding, minimizing effects on normal T cells, Tregs, or systemic toxicity.

It was recently demonstrated that IL-2 induces an alternative differentiation pathway of T cells, resulting in the generation of distinct “better effector” CD8⁺ T cells (Hashimoto et al., Nature 610(7930):173-181 (2022)). This process may rely, at least in part, on IL-2 binding to IL-2Rα. Additionally, IL-2Rβγ-biased agonists may drive T cells towards a terminally differentiated state (Codarri et al., Nature 610(7930):161-172 (2022)). CAR-engagers may be able to overcome the need for IL-2Rα in the alternative differentiation pathway by anchoring the low-affinity IL-2 on the surface of the CAR T cells via the antigen-to-CAR binding, thereby promoting the generation of memory CAR T cells. A potential synergistic effect between CAR signaling and IL-2 signaling may also exist.

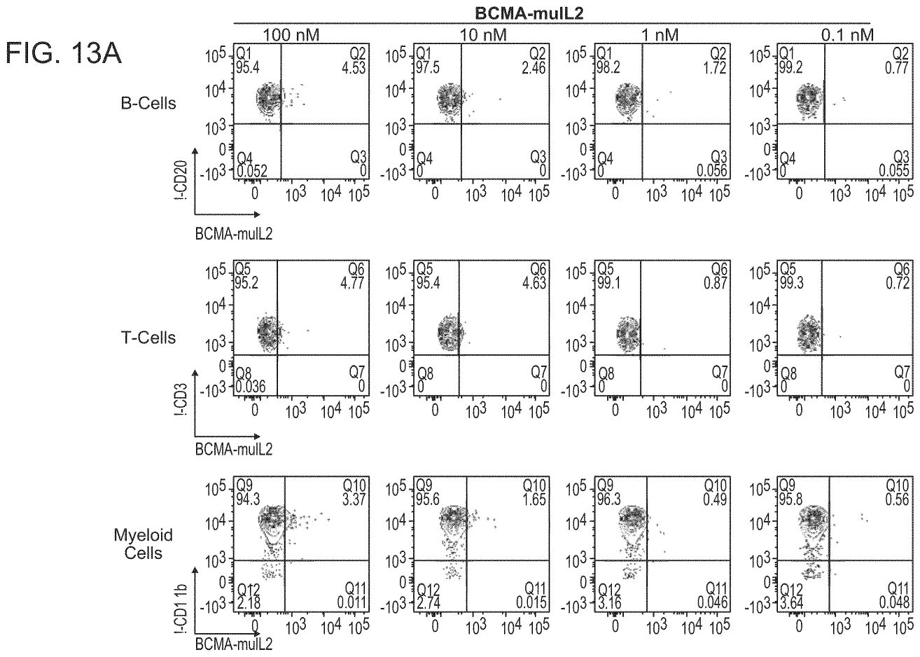

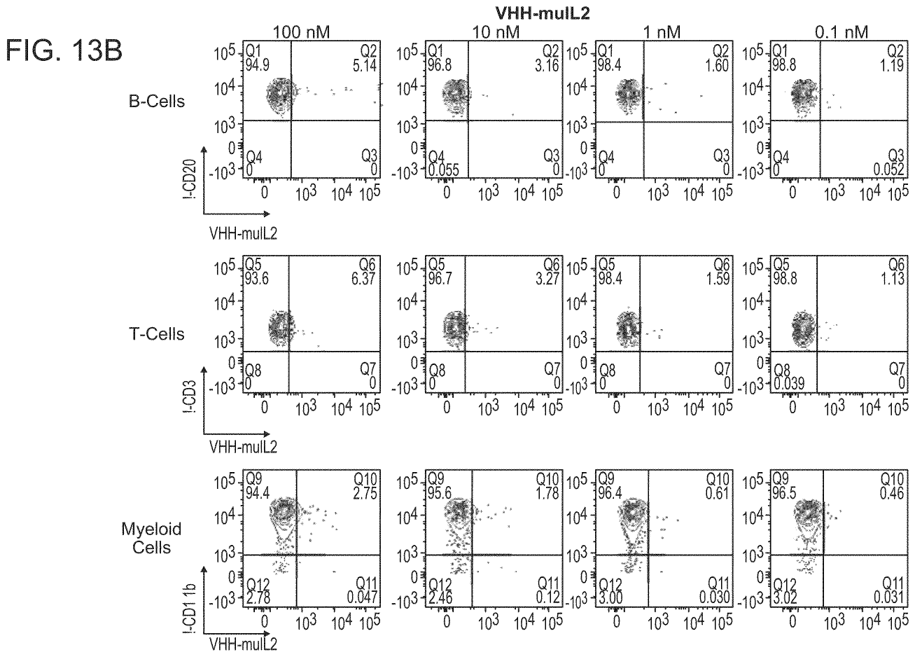

To assess the binding affinity of BCMA-containing CAR-engagers, flow cytometric analysis was performed by staining BCMA CAR T cells with varying concentrations of the BCMA CAR-engagers. An EC₅₀ of about 0.21 nM was observed for the BCMA CAR-engager, which was comparable to that of a dimeric BCMA lacking the muIL2 (BCMA-CH3) (FIG. 2E), indicating binding was mainly due to the BCMA ectodomain rather than the muIL2. FIG. 2E shows the dose-dependent staining of BCMA CAR T cells with the CAR-engager using flow cytometry (n=3 for each point), non-transduced T cells were used as controls, a secondary Alexa647-labeled anti-FLAG antibody was used for staining. Error bars represent mean with 95% confidence interval. Minimal binding of BCMA CAR-engager was observed to non-transduced T cells as well as minimal binding of VHH-muIL2, a control construct which replaces the BCMA ectodomain with an irrelevant nanobody (VHH) in the CAR-engager CH3-muIL2 construct. This observation further suggests that the binding of the CAR-engager to CAR T cells is primarily driven by the BCMA antigen, and that the muIL2 exhibits weak binding to both CAR T and non-transduced T cells. The BCMA-muIL2 CAR-engager did not exhibit binding to any immune cell populations in human peripheral blood mononuclear cells (PBMCs) (FIGS. 13A-13B).

Next, the functional effects of BCMA CAR-engager on BCMA CAR T cells were evaluated. After a 24-hour resting period without cytokines, BCMA CAR T cells were incubated with varying concentrations of the BCMA-muIL2 CAR-engager for 24 h, followed by assessment of the expression of the CD69 activation marker (Cibrián and Sánchez-Madrid, Eur. J. Immunol. 47(6):946-953 (2017)) using flow cytometric analysis. The results demonstrated a dose-dependent and selective increase in CD69 expression on CAR T cells (FIG. 2F), while no effect was observed on non-transduced T cells (FIG. 2G). Moreover, BCMA CAR-engager treatment resulted in a significantly higher increase in CD69 expression compared to VHH-muIL2, BCMA-CH3, or their combination suggesting the observed effect is only evident when the low-affinity IL-2 is fused to the antigen. An unpaired t-test indicated a statistically significant (P<0.0001) increase in activation in the BCMA-muIL2 treatment group compared to VHH-muIL2, BCMA-CH3, or their combination control groups at a concentration of 0.1 nM or higher (error bars represent mean with 95% confidence interval) (FIG. 2F). Zero treatment and CD3/CD28 activation in FIG. 2G were used as negative and positive controls, respectively (error bars represent mean with 95% confidence interval).

BCMA CAR-E does not inhibit killing efficacy of BCMA CAR T-cells. Since both the CAR-engagers and cancer antigens bind the CAR, the potential inhibitory effect of BCMA CAR-engager on the killing activity of BCMA CAR T cells was investigated. To investigate, a killing assay was conducted using BCMA CAR T cells and patient-derived BCMA⁺ OPM2 cancer cells in the presence of varying concentrations of the BCMA CAR-engager. Remarkably, the results demonstrated no inhibition of killing even at the highest tested concentration (100 nM of the CAR-engager) (FIG. 2H). OPM2 cells were co-incubated with BCMA CAR T cells (shown in red) or non-transduced T cells (shown in gray) (E:T ratio 1:1; 30,000 cells of each) in the presence of varying concentrations of the BCMA-muIL2 CAR-E treatment. Live (PI⁻) OPM2 cells were counted 48 hours later, with an N of 3 for each of the experiments. Error bars in FIG. 2H represent mean with standard deviation. Without being bound by theory, this finding might be attributed to the reversibility of CAR-engager binding to CAR, while the killing process, which involves the clustering effect and synapse formation between CAR and cancer antigen, is an irreversible event. Moreover, the binding avidity of CAR to membrane-bound BCMA might surpass the CAR binding avidity to the soluble antigen, contributing to this result. Notably, multiple myeloma patients exhibit high levels of soluble BCMA in their circulation due to shedding caused by γ-secretase (Laurent et al., Nat. Commun. 6:7333 1-12 (2015)). Despite this, BCMA CAR T cells produce remarkable initial responses in patients suggesting that the soluble BCMA antigen does not inhibit the activity of CAR T cells. The experimental findings disclosed herein are consistent with these earlier findings.

The BCMA CAR-engager selectively induces STAT5 activity in CART cells through the cis-delivery of the low-affinity IL-2 to the same targeted CAR T cells. IL-2 is known to exhibit strong activity on T cells, and the phosphorylation of STAT5 both serves as a reliable indicator of IL-2/IL-2R engagement and correlates with downstream effects such as phenotypic marker expression and cell proliferation (Jones et al., J. Immunol. 205(7):1721-1730 (2020)). To evaluate the impact of BCMA CAR-engager on STAT5 activity, BCMA CAR T cells were exposed to varying concentrations of the BCMA CAR-engager. Following a 5-minute incubation at 37° C., cells were fixed and stained for pY694 STAT5. The results revealed that the BCMA-muIL2 CAR-engager induced phosphorylation of STAT5 in CAR T cells with an EC₅₀ of ~0.014 nM (FIG. 2I). In contrast, the VHH-muIL2 control required a higher concentration (EC₅₀=3.9 nM) to induce STAT5 phosphorylation, indicating that the BCMA-mediated delivery of the low-affinity IL-2 to CAR T cell surface significantly enhances the sensitivity of the muIL-2 by over 200-fold. Wild-type IL-2 exhibited a lower EC₅₀ (approximately 0.001 nM) suggesting a difference in signaling kinetics. The two-step process involved in STAT5 activity mediated by the BCMA-muIL2 CAR-engager involves: (i) binding of the antigen-to-CAR on T cell surfaces and (ii) subsequent interaction of the low-affinity IL-2 with nearby IL-2R, which, without being bound by theory, may be the reason for the measured difference. In contrast, wild-type IL-2 requires only binding to IL-2R, enabling it to more rapidly induce STAT5 activity. The VHH-muIL2 can activate STAT5 solely through the low-affinity IL-2, which may explain its requirement for higher concentrations to induce STAT5 activity in T cells.

BCMA CAR T cells pre-blocked with BCMA-CH3 showed a significant decrease in pSTAT5 levels to the same degree as control VHH-muIL2, validating that the potency of the CAR-engager is mediated by antigen-to-CAR binding (FIG. 2I). To determine whether the CAR-engager binding to target cell can result in STAT5 signaling in an adjacent cell (trans-activation), non-blocked and pre-blocked BCMA CAR T cells were co-cultured in the presence of varying concentrations of CAR-engager. Pre-blocked CAR T cells had lower pSTAT5 levels compared to their co-cultured non-blocked CAR T cells, indicating that CAR-engager affects the targeted CAR T cells (cis-activation) but not adjacent cells. BCMA CAR T cells were treated with the indicated treatments for 5 min at 37° C. followed by STAT5 phosphorylation assessment. For the pre-blocking experiments, the BCMA CAR T cells were treated with BCMA-CH3 (100 nM) for 20 min at 4° C. prior to the 5 min exposure to the CAR-engager treatment at 37° C. (n=3 for each condition). Error bars in FIG. 2I represent mean with standard deviation. Taken together, the analysis of STAT5 activity supports the notion that the BCMA CAR-engager exerts its influence on targeted CAR T cells through the cis delivery of the low-affinity IL-2, with the effect mediated via antigen-to-CAR binding.

### Example 3: The CAR-Engager Immune Cell Effector Domain Stimulates T Cells Independent of a CAR

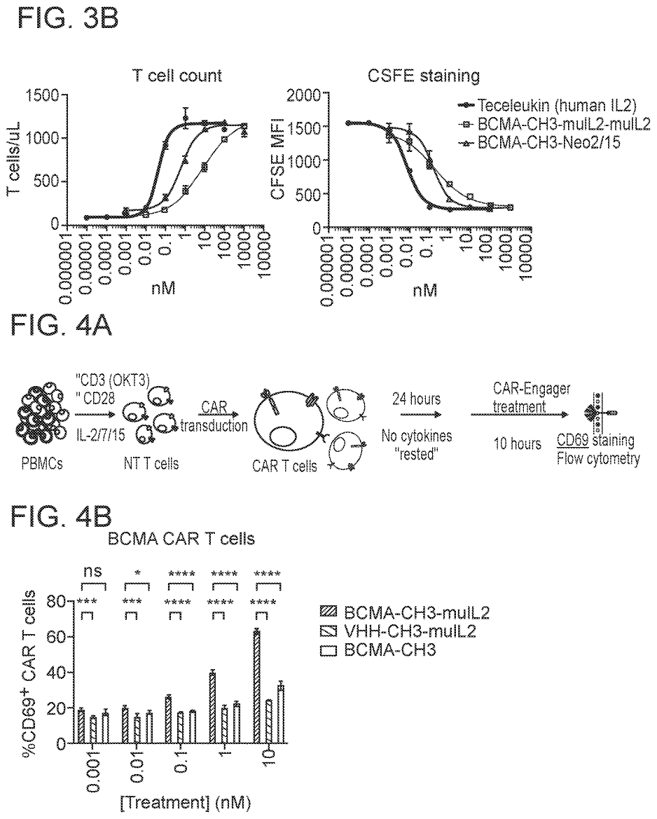

To show that CAR-engager immune cell effector domains stimulate immune cells, the following experiment was performed. In this experiment, the experimental setup of which is illustrated in FIG. 3A, peripheral blood mononuclear cells (PBMCs) were stimulated with anti-CD3, anti-CD28, IL-2, IL-7, and IL-15 to produce activated T cells, which were not transduced with any exogenous transgenes. The activated T cells were treated with teceleukin (recombinant human IL-2 without glycosyl units), a CAR-engager that contains a N-terminal ectodomain that binds BCMA (~7 kDa), a CH3 domain (~14 kDa), and two repeats of the weak affinity variant of IL-2 immune cell effector domain, with the overall structure of BCMA-CH3-muIL2-muIL2 and referred herein as BCMA-muIL2, or a CAR-engager that contains an ectodomain that binds BCMA, a CH3 domain, and the Neo-2/15 immune cell effector domain, with the overall structure of BCMA-CH3-Neo-2/15, referred herein as BCMA-Neo-2/15, for 4 days. After treatment, T cells were counted and stained with carboxyfluorescein succinimidyl ester (CFSE) and analyzed for mean fluorescence intensity (MFI) of CFSE to determine T cell division.

T cells treated with the CAR-engagers BCMA-muIL2 or BCMA-Neo-2/15 effected T cell counts and division (CFSE staining), similar to teceleukin, which is known to activate T cells; however, the CAR-engager treatment resulted in less sensitivity as compared to the teceleukin treatment. Systemic administration of IL-2 is associated with severe side effects (Rosenberg, J. Immunol. 192(12):5451-5458 (2014); Dutcher et al., J. Immunother. Cancer. 2(1):26 1-23 (2014); Pachella et al., J. Adv. Pract. Oncol. 6(3):212-221 (2015)), including vascular leak syndrome and preferential expansion of CD4⁺CD25⁺ regulatory T (Treg) cells, that are known to result in immune suppression. The presently disclosed results indicate that the CAR-engagers do not activate normal T cells when used at low concentrations, and that the stimulatory immune cell effector domains retain their normal function when attached to an ectodomain of a CAR-engager to activate T cells.

### Example 4: CAR-Engagers Activate CAR T Cells Specifically Through the Ectodomain

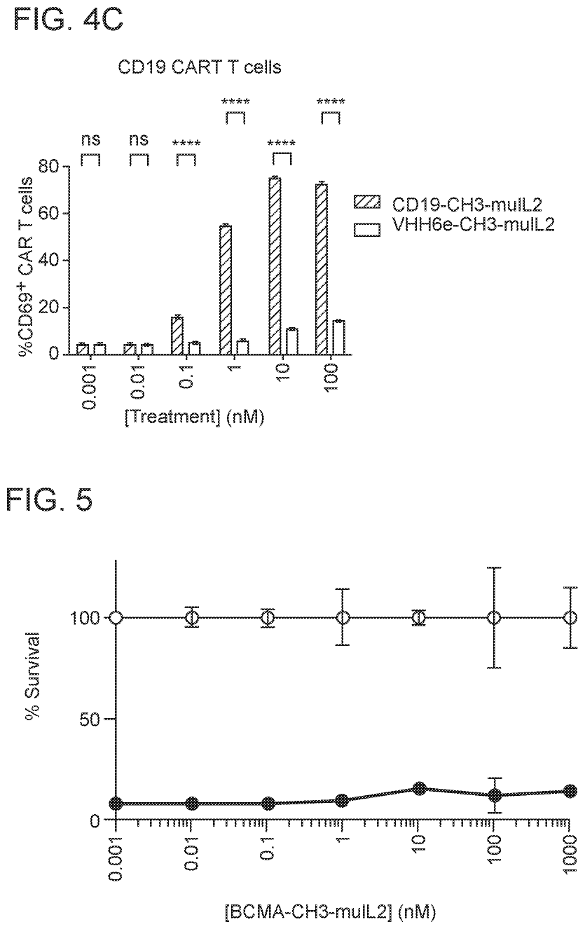

To show that CAR-engager immune cell effector domains stimulate immune cells, the following experiment was performed. As illustrated in FIG. 4A, PBMCs were stimulated with anti-CD3, anti-CD28, IL-2, IL-7, and IL-15 to produce activated T cells, which were then transduced with a vector containing a CAR. The activated CAR-expressing T cells (CAR T cells) were rested for 24 hours and then treated with CAR-engagers containing an immune cell effector domain or CAR-engagers lacking an immune cell effector domain as an ectodomain control.

CAR-engagers containing BCMA ectodomain, a CH3 domain, and containing either 4-1BBL (BCMA-41BBL), weak affinity IL-2 (BCMA-muIL2), or Neo-2/15 (BCMA-Neo-2/15) immune cell effector domains were tested for T cell activation. To test the ectodomain specificity of the CAR-engagers for CAR T cells, as a control, irrelevant nanobody that binds the intracellular protein UBC6E (VHH6E) fused to a CH3 domain, and either 4-1BBL (VHH6E-41BBL) or weak affinity IL-2 (VHH6E-muIL2) were tested for T cell activation. An additional ectodomain specificity control containing a nanobody that binds FN1 (clone NJB2, abbreviated NJB2-VHH) fused to a CH3 domain, and the Neo-2/15 stimulatory (NJB2-VHH-Neo-2/15) was tested for T cell activation. The ectodomain specificity controls have similar overall structure as the CAR-engagers used in this experiment (protein domain-CH3-muIL2-muIL2 or protein domain-CH3-Neo-2/15). Ectodomain specificity controls and CAR-engagers were incubated with CAR T cells for 10 hours, and the cells were stained for CD69 as an activation marker and measured by flow cytometry.

All of the BCMA ectodomain CAR-engagers induced expression of CD69 in CAR T cells (FIG. 4B). The BCMA-CH3-Neo-2/15 had the lowest threshold of induced expression of CD69 in CAR T cells (0.01 nM CAR-engager). The BCMA-CH3 protein (lacking an immune cell effector domain) had minimal effect on CAR T cell expression of CD69 at the highest concentration tested, 10 nM of BCMA-CH3 protein. None of the ectodomain specificity control proteins induced CAR T cell expression of CD69. These results indicate that CAR-engagers containing a cancer antigen ectodomain specifically activates CAR T cells that expresses a CAR that recognizes CAR-engager's cancer antigen ectodomain.

### Example 5: CAR-Engagers Stimulate CAR T Cell Killing Target Cells

To show that CAR-engagers do not inhibit CAR T1 cell killing, the following experiment was performed. CAR T cells were produced as described above and co-incubated with CAR-engagers and BCMA⁺ multiple myeloma cancer cells. CAR T cells were incubated with OPM2 BCMA⁺ cells at an E:T ratio of 1:1 for 1 day and analyzed for target cell survival as compared to target cells without T cell coincubation (FIG. 5A).

BCMA ectodomain CAR-engagers with either a muIL2 (BCMA-CH3-muIL2) or a 4-1BBL (BCMA-CH3-41BBL) immune cell effector domain did not inhibit killing of OPM2 cells (FIG. 5B). These results indicate that CAR-engagers containing a cancer antigen ectodomain and a stimulatory immune cell effector domain do not inhibit CAR T cell killing of target cells that also express the same cancer antigen as the CAR-engager.

### Example 6: CAR-Engagers Reduce Tumor Burden, Extend Survival, and Extend CAR T Cell Persistence In Vivo

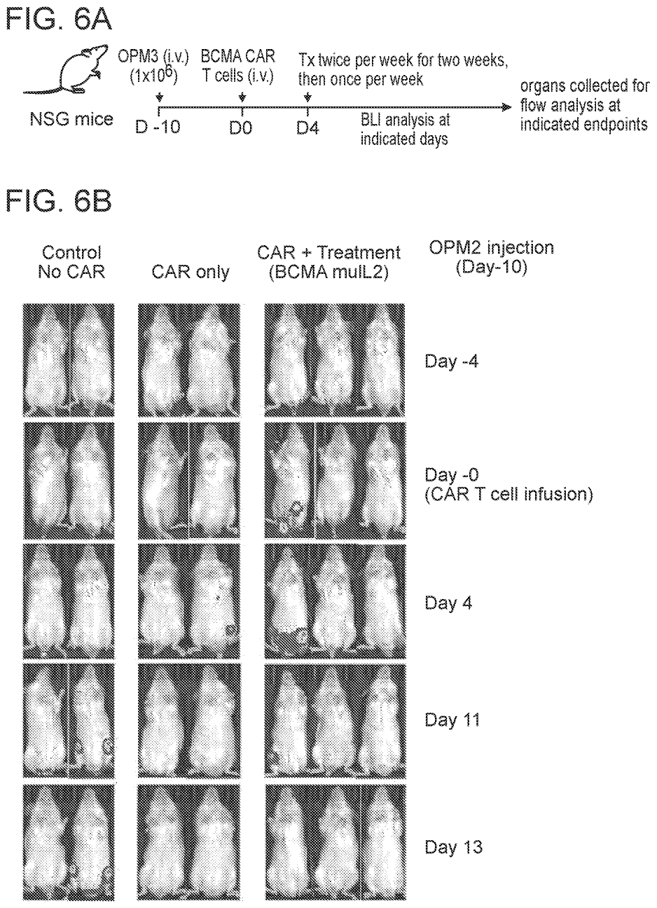

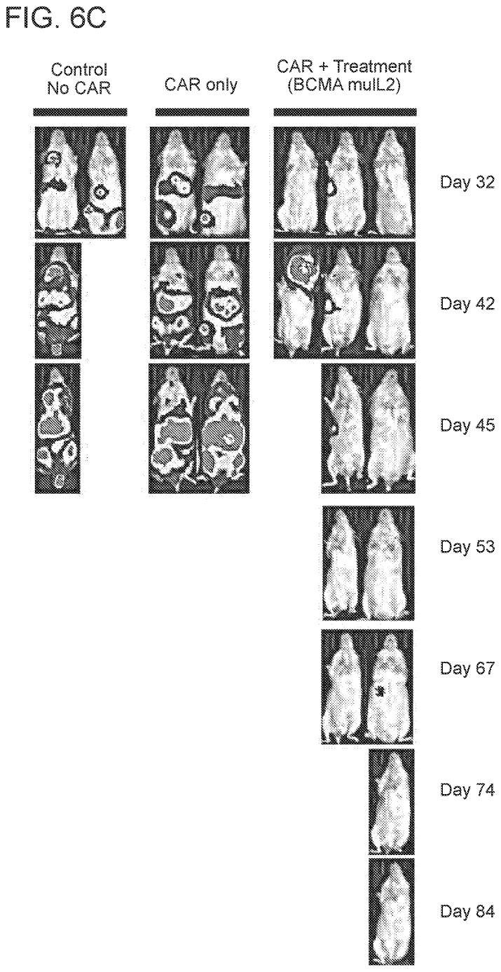

To show that CAR-engagers reduce tumor burden, extend CAR T cell in vivo persistence, and extend survival, the following experiment was performed. 1×10⁶ OPM2 BCMA⁺ multiple myeloma cancer cells were intravenously (i.v.) injected into NOD-scid IL2Rγ^{null} (NSG) mice 10 days before infusing a suboptimal dose of 5×10⁵ anti-BCMA CAR T cells by i.v. injection. After CAR T cell infusion, mice were treated twice weekly for two weeks, followed by once weekly with 200 μg/mouse a CAR-engager containing a BCMA ectodomain, a CH3 domain, and two weak affinity IL-2 immune cell effector domains (BCMA-CH3-muIL2-muIL2) by intraperitoneal (i.p.) injection (FIG. 6A).

Mice were subjected to bioluminescent imaging (BLI) for luciferase (indicating tumor burden of luciferase+OPM2 cells) on days indicated in FIGS. 6B-6D. Control mice that received OPM2 cells and no-CAR T cell infusion had progressively more tumor burden during the experiment and reached a humane end point on day 39 and 46. Mice that received OPM2 cells and the suboptimal dose of CAR T cells had controlled tumor growth until day 32, when they also saw progressively more tumor burden during the experiment and reached a humane end point on day 46. Mice that received OPM2 cells, CAR T cells, and CAR-engager therapy had reduced tumor burden (FIGS. 6B-6D). In the CAR-engager treated group, all mice completely cleared OPM2 tumor cells from the bone marrow, as no signal was detected by imaging. One mouse in this group had significant OPM2 cell growth, due to formation of a solid tumor close to the eye and reached a humane end point on day 42. The remaining two mice completely cleared OPM2 tumor cells, as no signal was detected by imaging and survived the experiment.

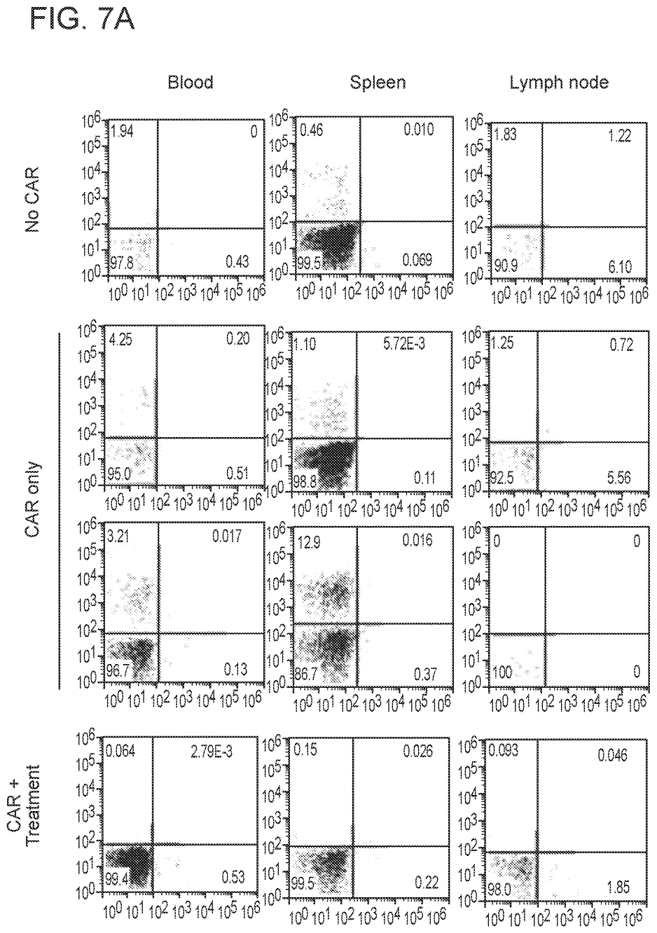

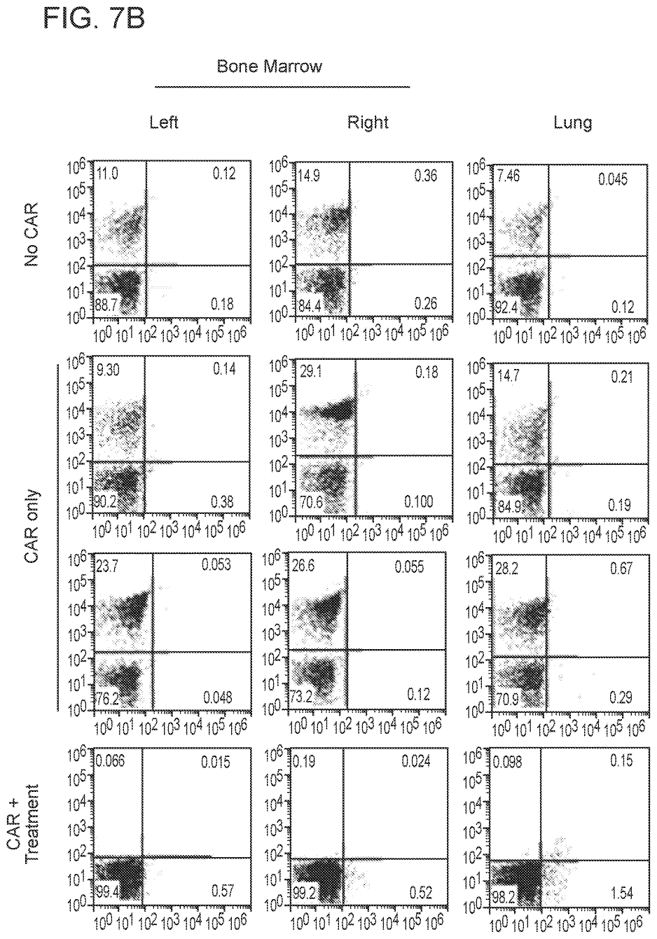

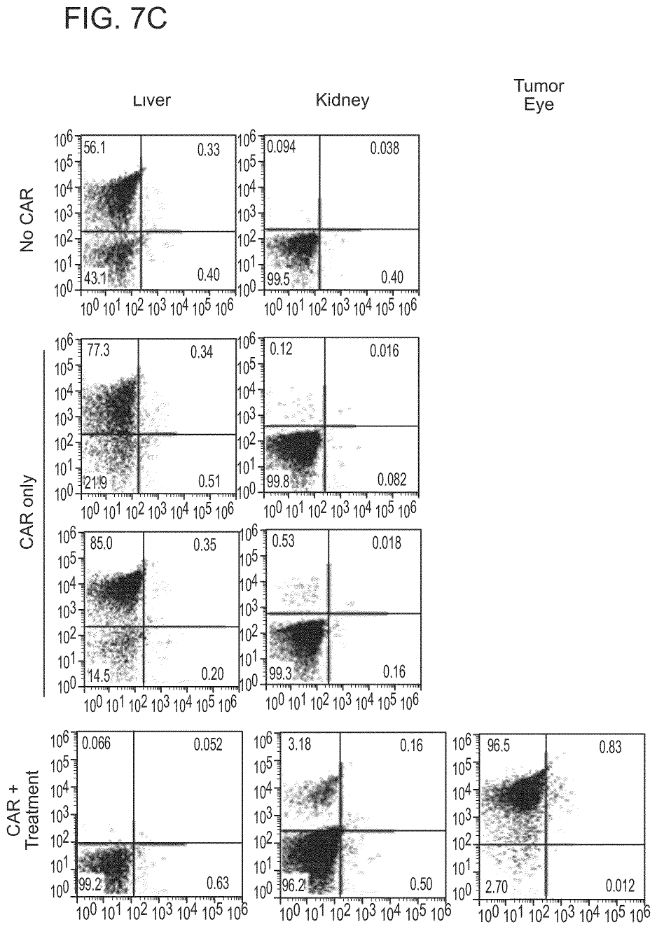

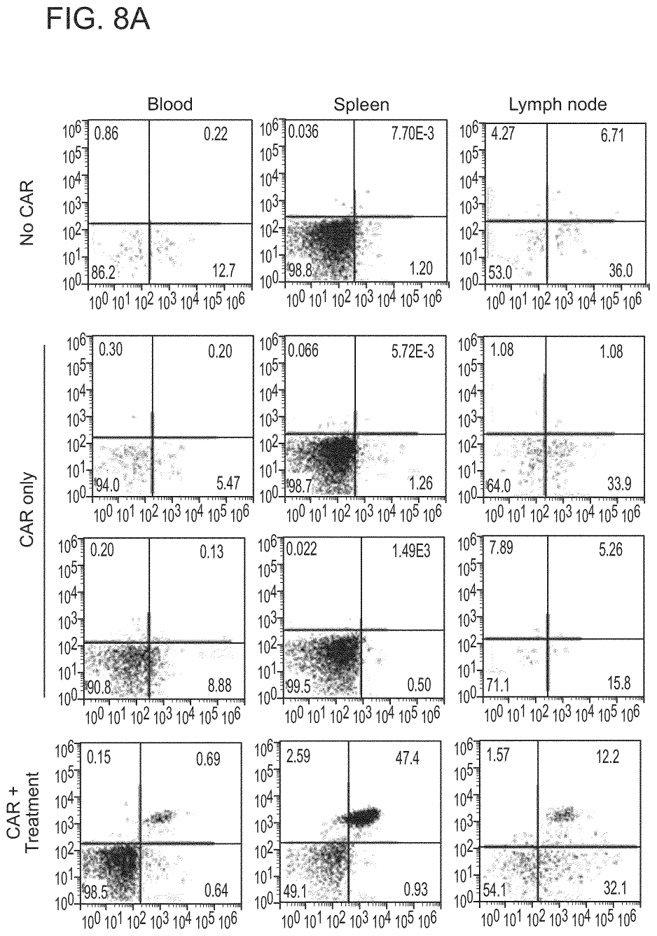

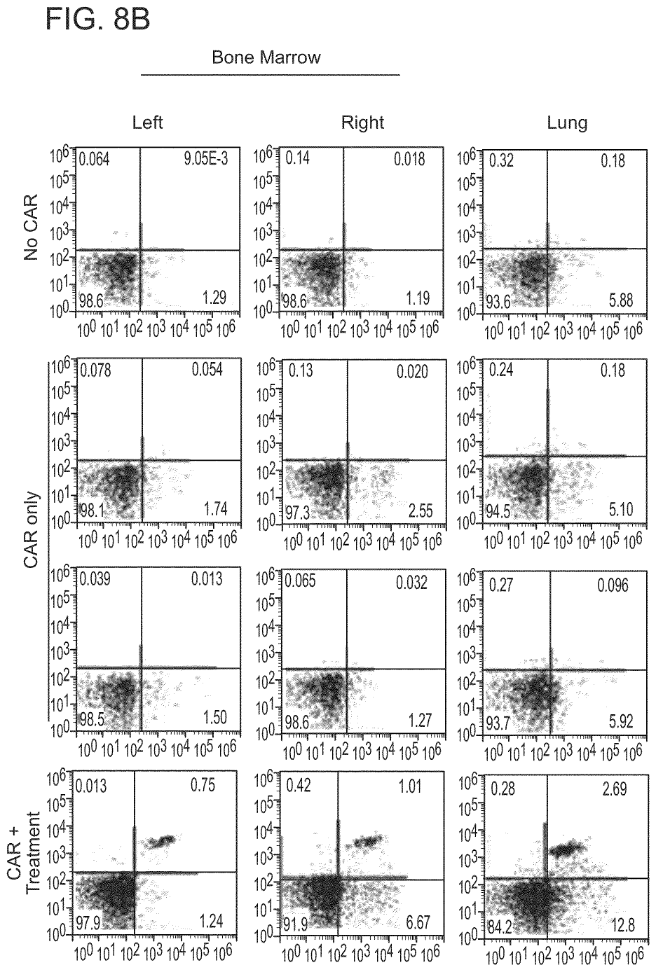

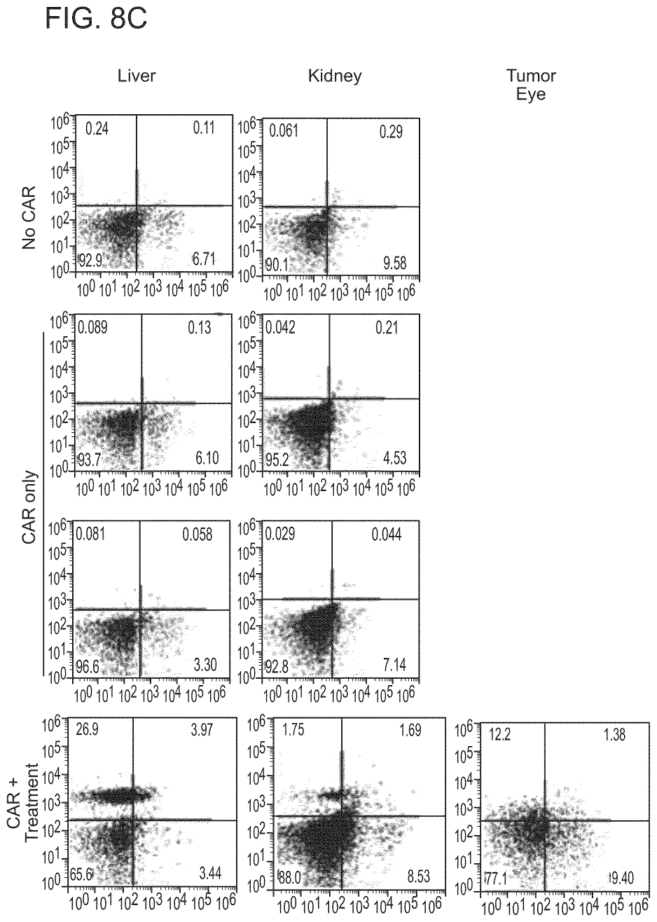

Next, the in vivo persistence of OPM2 and CAR T cells were analyzed in these mice by flow cytometry. One control mouse (OPM2 cells with no-CAR T cell infusion) was sacrificed on day 46, two CAR-only mice were sacrificed on day 46, and one CAR T cell and CAR-engager treated mouse was sacrificed on day 42. Sacrificed mice were analyzed for GFP+OPM2 cells (FIGS. 7A-7C) and CD45⁺ CAR⁺ T cells (FIGS. 8A-8C) in the blood, spleen, lymph node, bone marrow and lung. The CAR T cell and CAR-engager treated mouse was also analyzed for GFP+OPM2 cells and CD45+ CAR⁺ T cells in the eye tumor site.

FIGS. 7A-7C show flow cytometry with GFP on the y-axis. GFP⁺ OPM2 cells were detected at similar levels in the bone marrow, lung (FIG. 7B), and liver (FIG. 7C) of the no-CAR control mouse and the CAR-only mice. One CAR-only mouse had significant levels of OPM2 cells in the blood and spleen (FIG. 7A).

The one mouse that received CAR T cell and CAR-engager treatment that developed an eye tumor had no to little OPM2 cells in the blood or spleen (FIG. 7A), bone marrow or lung (FIG. 7B), and liver (FIG. 7C). This mouse had more OPM2 cells in the kidney (3.18% of GFP⁺ cells), and the majority of cells in the eye tumor site were OPM2 cells (96.5% of GFP⁺ cells).

FIGS. 8A-8C show flow cytometry with anti-CD45 on the y-axis and BCMA⁺-CH3 tagged with Alexa Flour™ 647 (AF647) on the x-axis. CD45⁺ CAR⁺ T cells only persisted in CAR T cell and CAR-engager treated mice. CAR-only treated mice had little to no CD45⁺ cells in all organs tested (FIGS. 8A-8C). However, CAR T cell+CAR-engager treated mice had CD45⁺ cells that also stained positive for the BCMA cancer antigen (which is also the CAR binding target) tagged with AF647, as shown on the x-axis. CD45⁺ AF647⁺ double positive CAR T cells were detected in the blood, spleen, and lymph node (FIG. 8A), bone marrow and lung (FIG. 8B), and liver and kidney (FIG. 8C). CD45⁺ single positive cells were only detected in large numbers in liver and kidney (FIG. 8C). Little to no CD45⁺ AF647⁺ double positive CAR T cells were detected in the eye tumor site (FIG. 8C). These results indicated that CAR-engagers reduce tumor burden, extend CAR T cell in vivo persistence, and extend survival.

### Example 7: CAR-Engager Fate

The CAR-engagers bind CAR T cells at the cell surface at 4° C., and slowly internalize at 37° C. Internalization of CAR-engager was assessed using fluorescently labeled BCMA-muIL2 CAR-engager. BCMA CAR T cells were exposed to AlexaFluor647-labeled BCMA-muIL2, BCMA-CH3, or VHH-muIL2 at a concentration of 2 nM. The cells were incubated at either 4° C. or 37° C. for various time intervals, followed by fixation and subsequent microscopy imaging. It was observed that the control VHH-muIL2 underwent rapid internalization within 30 minutes at 37° C., whereas the internalization of BCMA-muIL2 CAR-engager was significantly slower (FIGS. 14A-14C). The internalization rate of BCMA-CH3 was similarly slow, even slower than that of BCMA-muIL2 CAR-engager. In FIG. 2C, the CAR and dsRed transcripts were encoded within the transgene, and thus the dsRed signal reflects the expression level of CAR. All imaged cells with mean intensities higher than background were reported. For the dsRed channel, the cytoplasm mean intensity was reported, as the dsRed is expressed inside the cell, whereas for the AlexaFluor 647 channel, the mean intensity for the entire cell was measured.

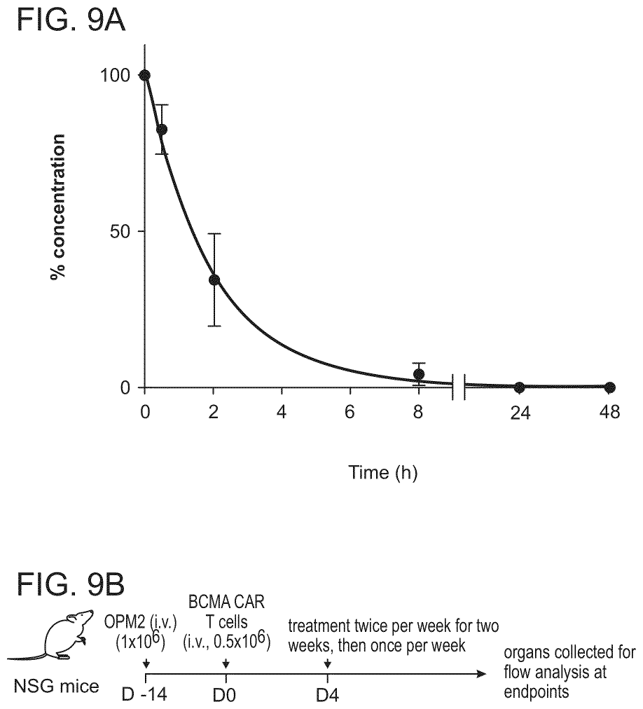

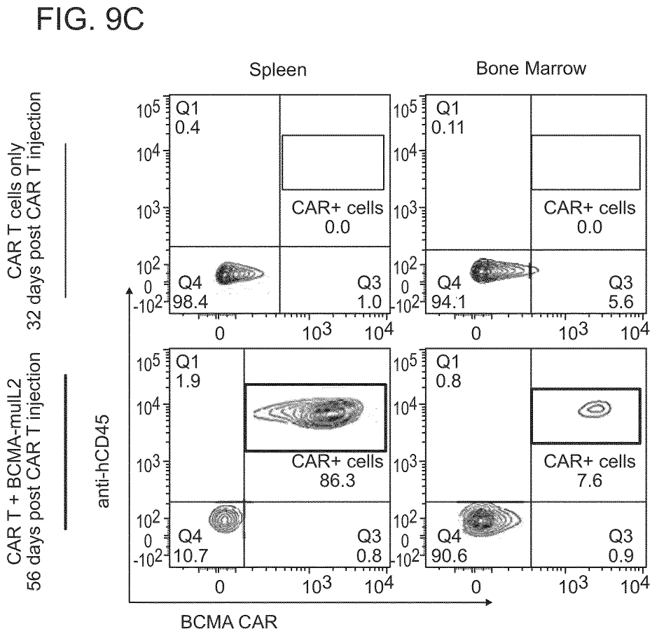

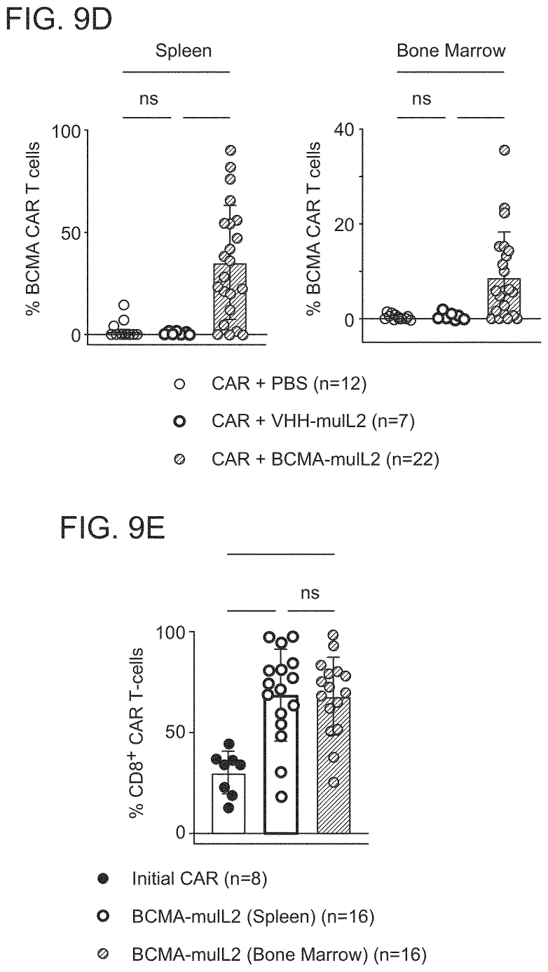

The CAR-engager rapidly clears from the circulation. Pulsing CAR T-cells with the CAR-engager treatment, where pulsing involves periods of stimulation followed by periods of resting, is superior to prolonged exposure to CAR-engagers as extended exposure can lead to exhaustion or the generation of terminally differentiated CAR T cells. A CAR-engager with a short circulation half-life can be more effective at expanding CAR T cells, driving generation of memory CAR T cells, decrease potential competition with tumor antigen for CAR binding, and enhanced safety profile in patients. Therefore, the CH3 domain of IgG1 was used in the CAR-engager platform. Pharmacokinetic studies illustrated that the circulatory half-life of the CAR-engager was short (1-1.5 hours) (FIG. 9A). NSG mice were administered 8 mg/kg of BCMA-muIL2 CAR-engager (delivered i.p., N=3 mice). Blood samples were collected via tail-vein puncture at five different time points (30 min, 2 h, 8 h, 24 h, 48 h) post-administration. The sera were then obtained by centrifugation and used for the subsequent analysis. An ELISA was performed to determine the concentration of the treatments in the sera. The ELISA plates were coated with 5 μg/ml anti-His6 antibody overnight, followed by incubation with the sera for 2 h at room temperature. An anti-FLAG HRP antibody was next used for the detection; the CAR-engager was engineered to have FLAG and His6 tags at the C-terminus. Based on the collected five time points, the initial concentration of the treatment in the sera was estimated to be 20% higher than the first (30 min) collected time point. The BCMA CAR-E was >90% and >99% cleared from the circulation in 8 h and 24 h, respectively. The circulating half-life was estimated to be about 1.5 hours for the BCMA CAR-engager. Error bars represent mean with standard deviation.

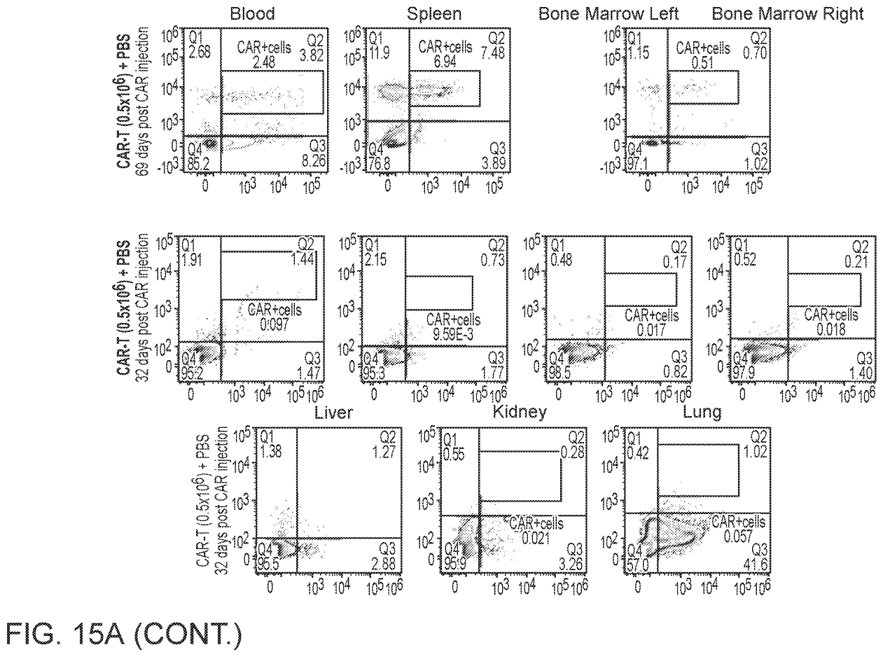

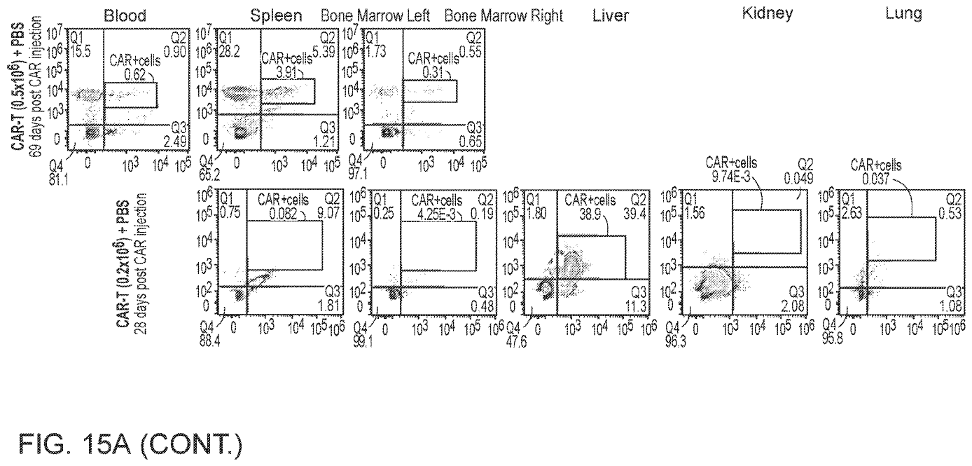

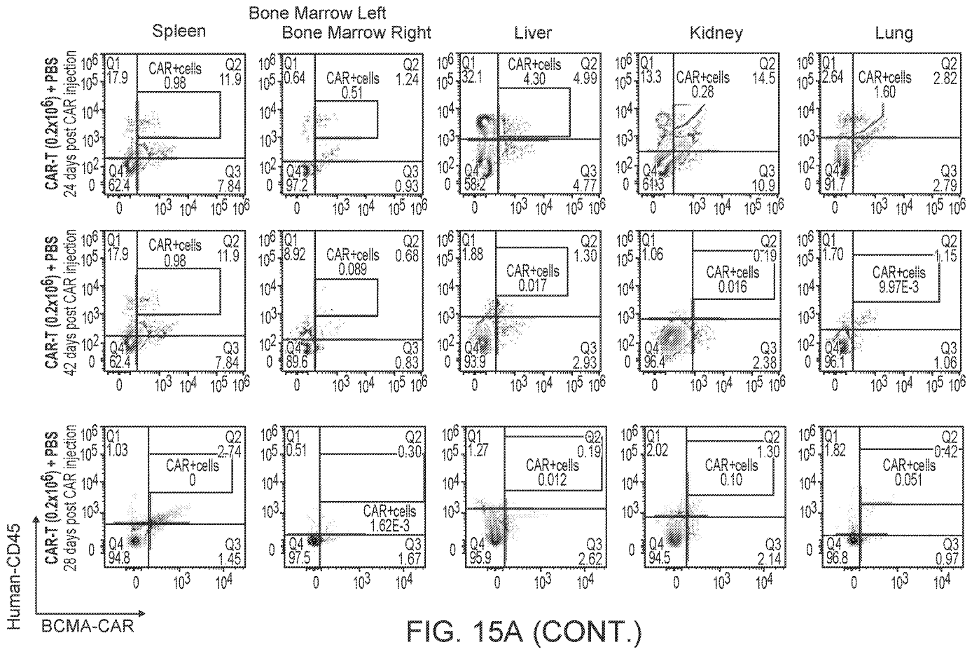

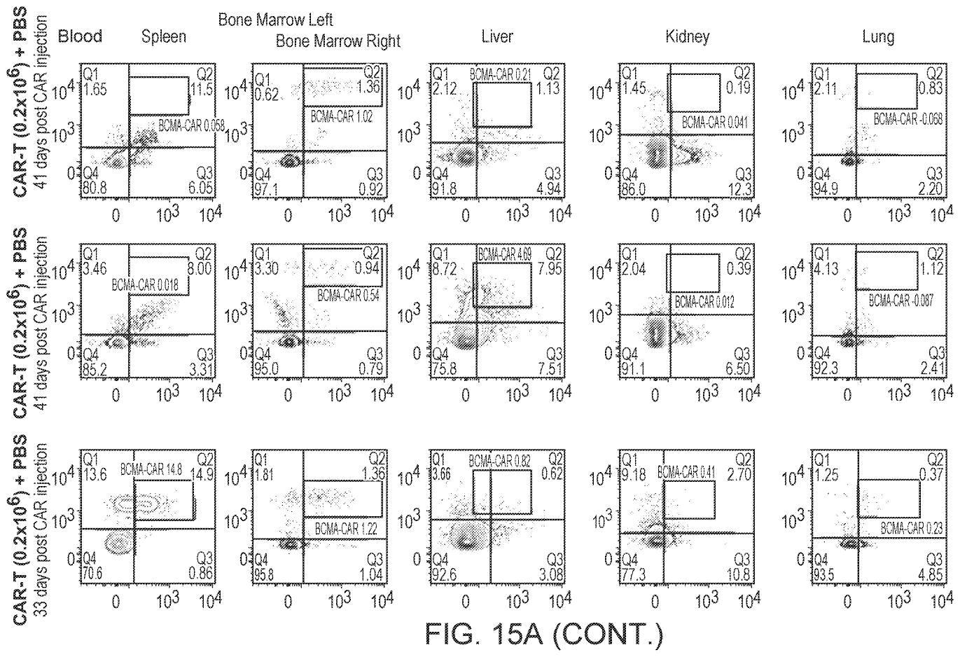

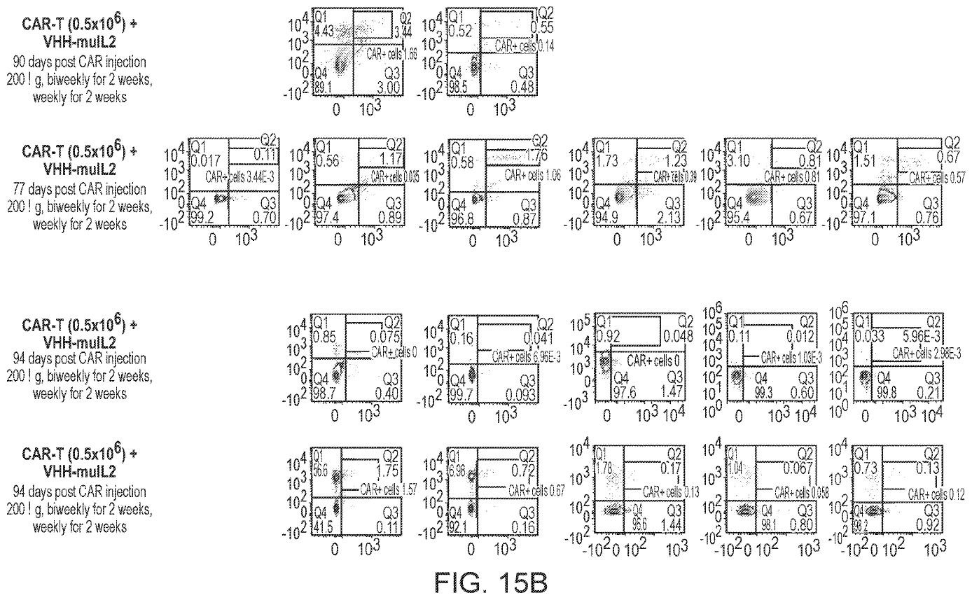

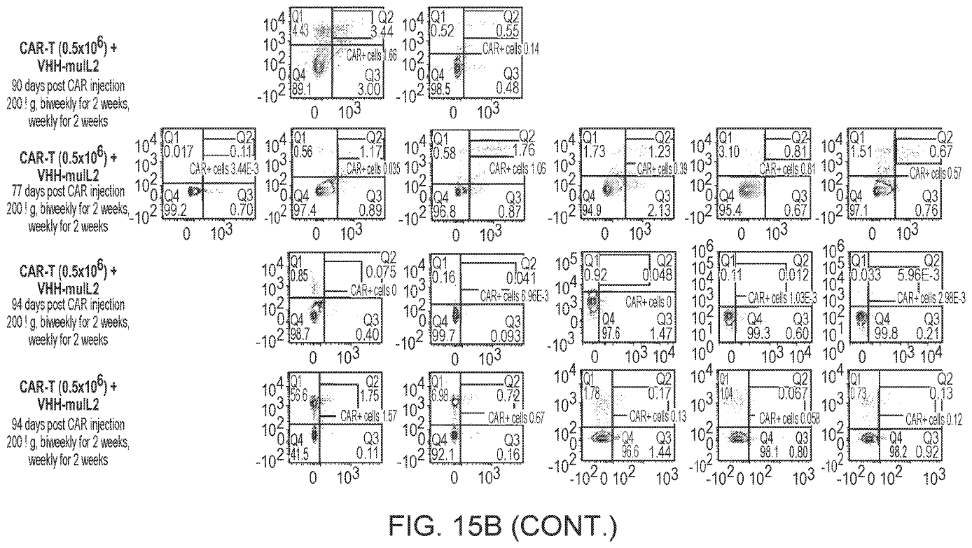

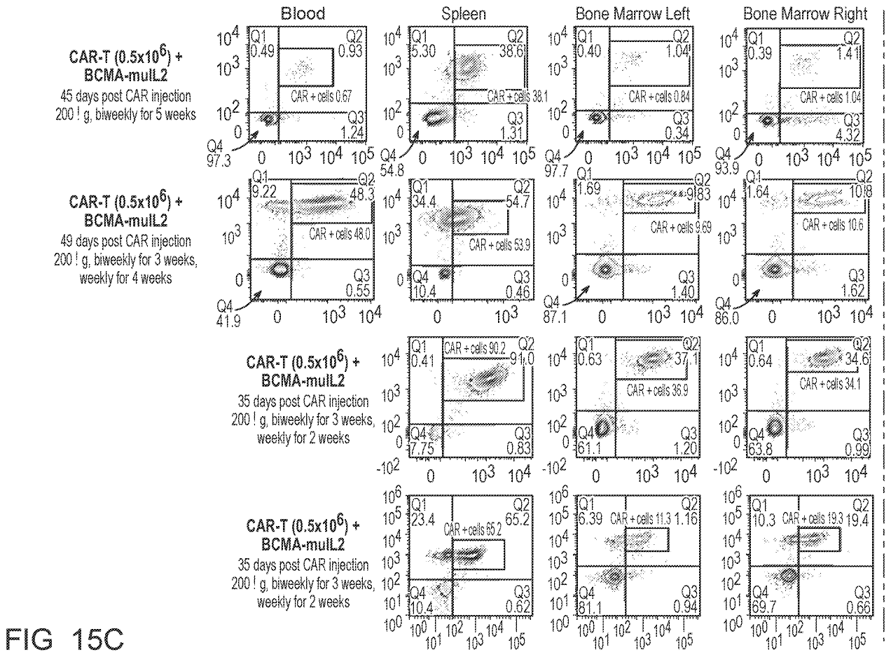

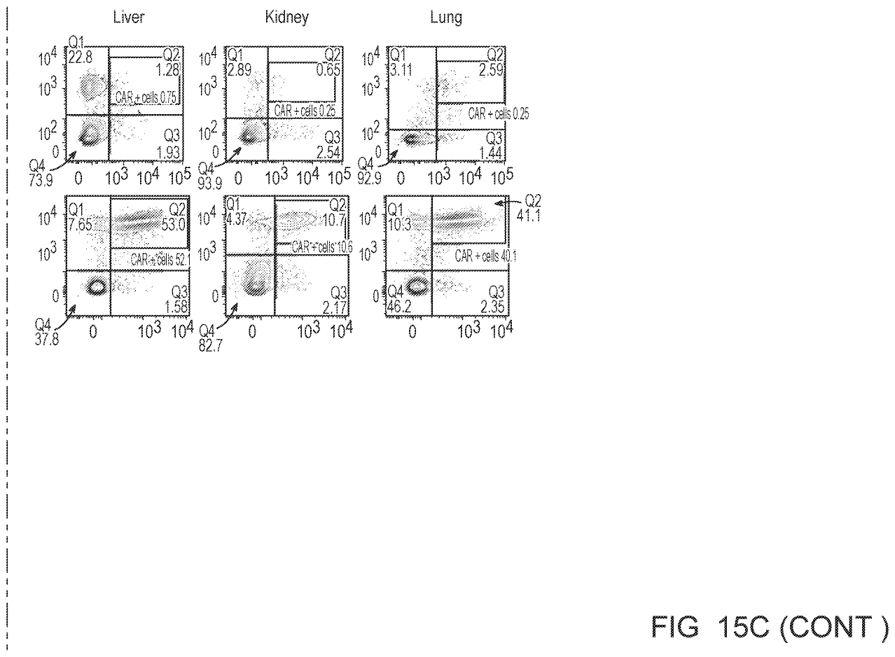

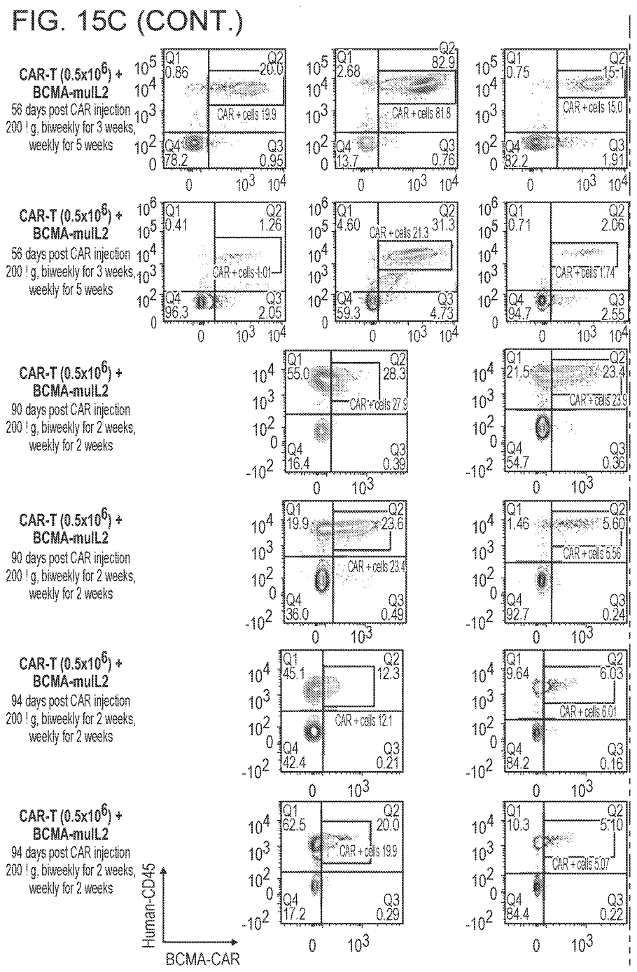

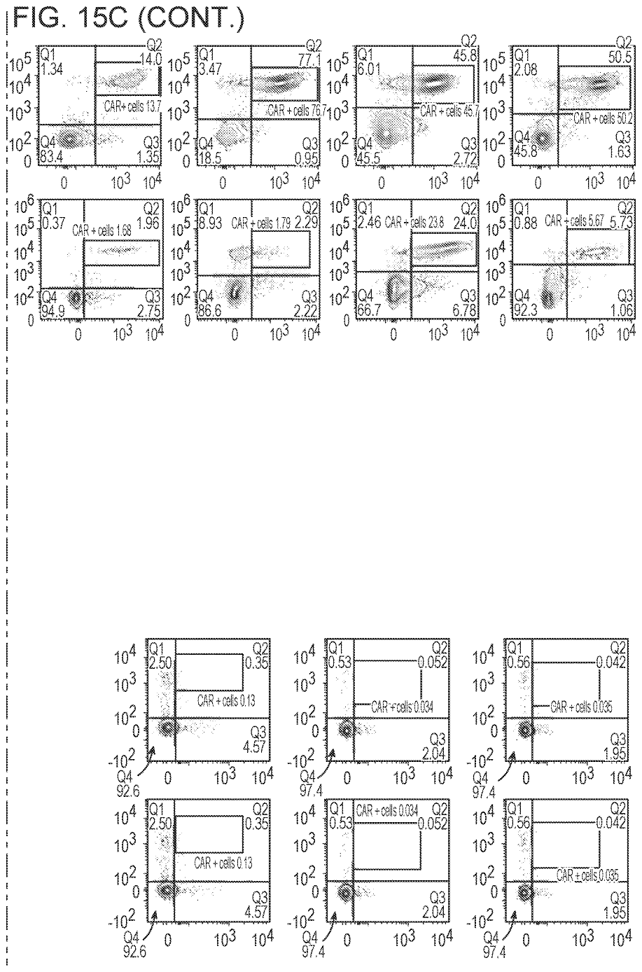

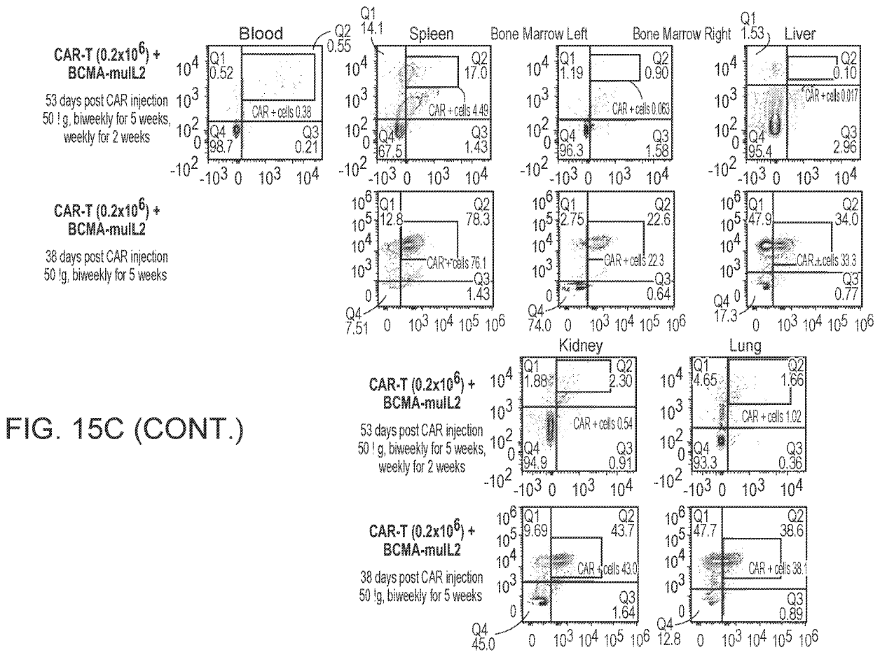

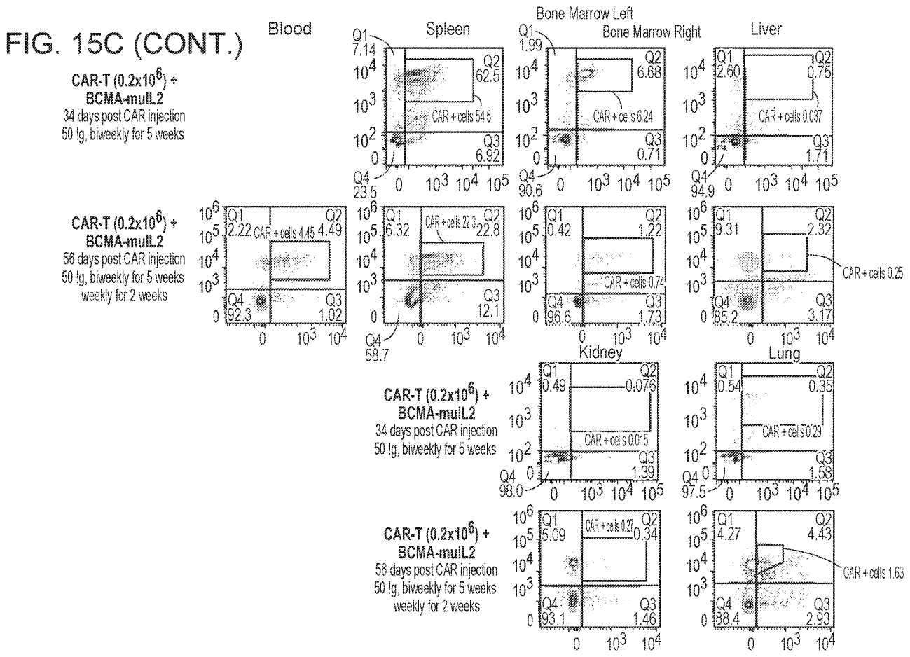

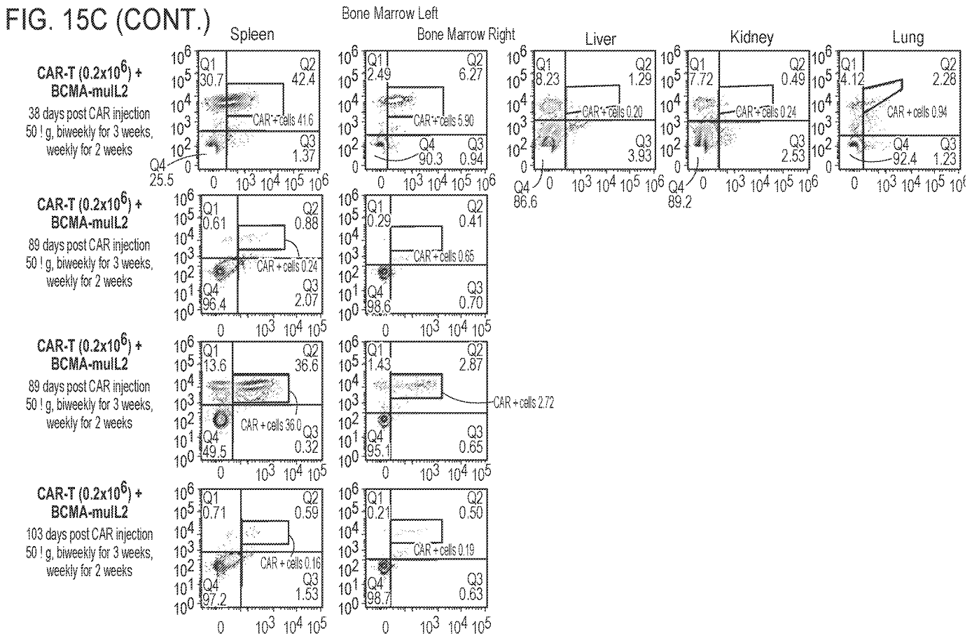

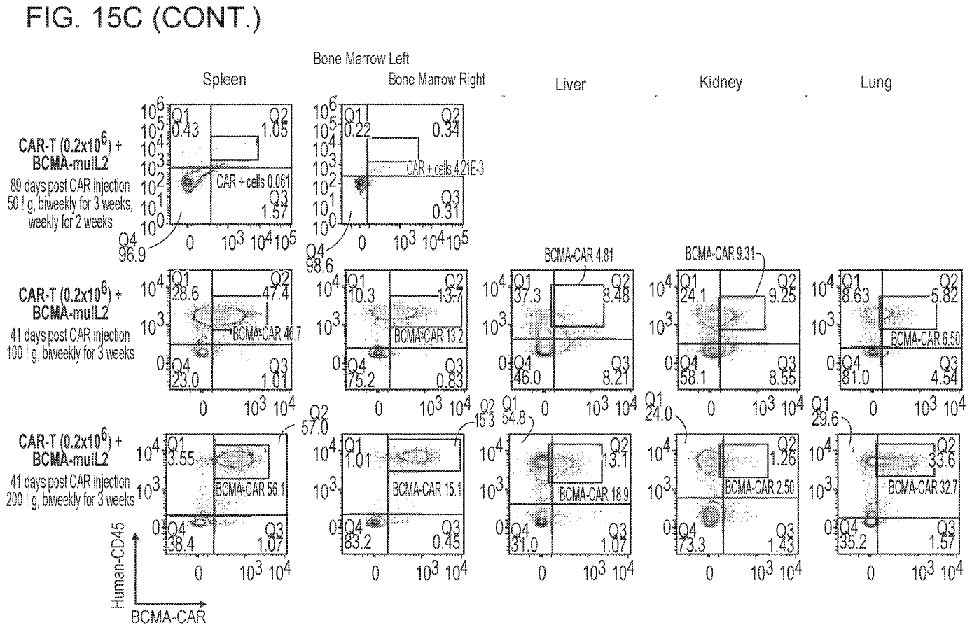

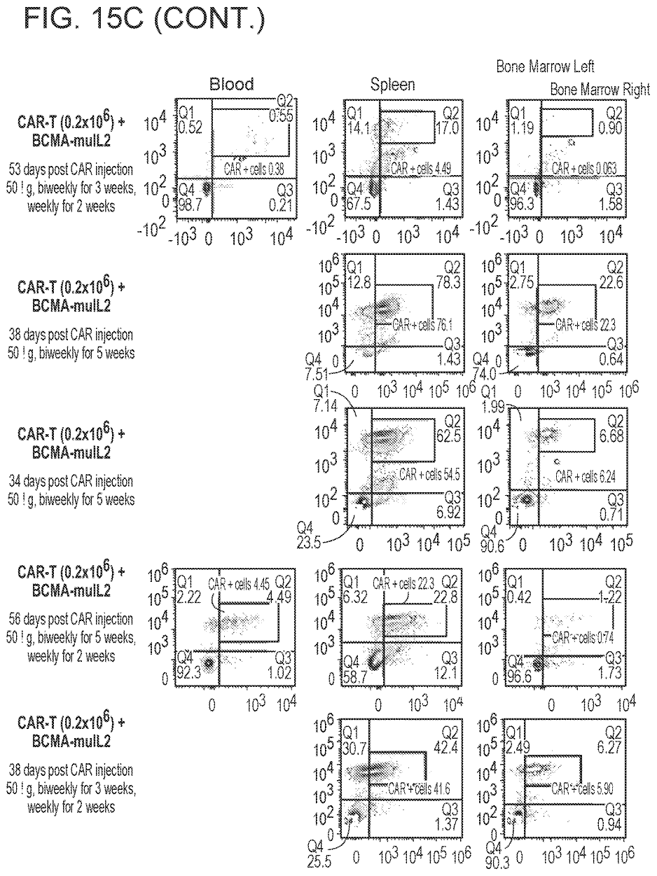

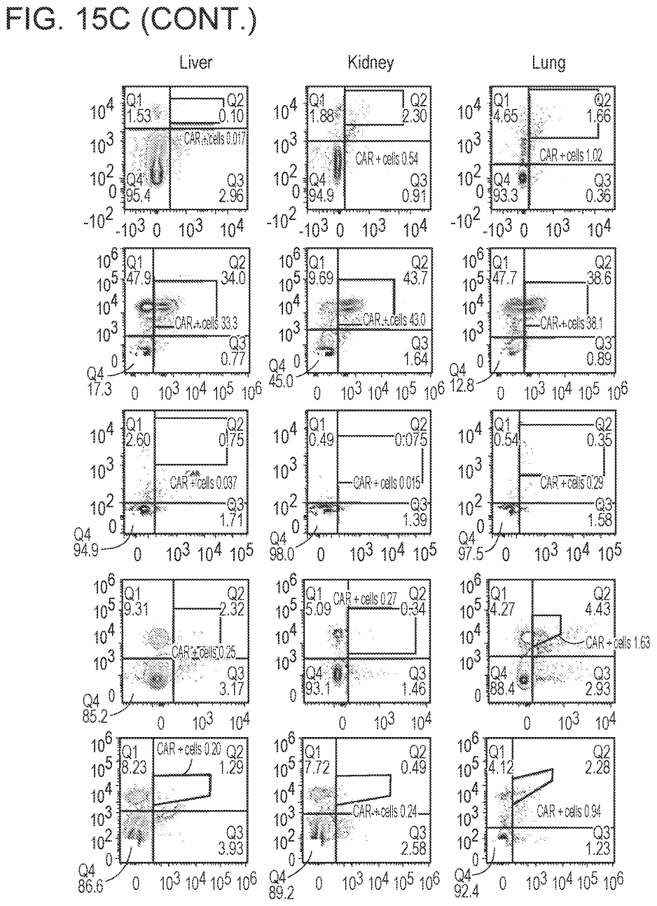

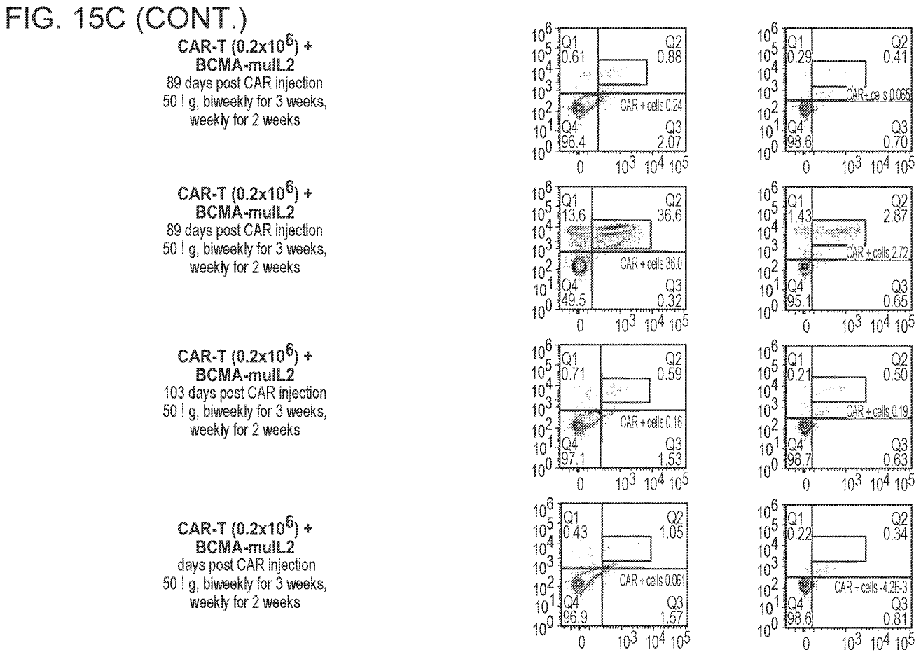

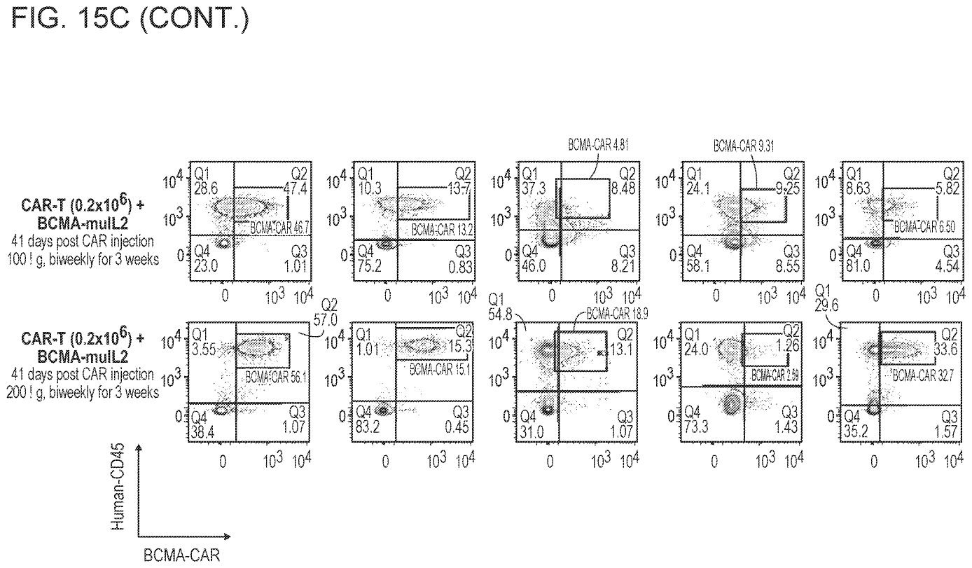

The BCMA CAR-engager enhances activity and persistence of CAR T-cells in a multiple myeloma (NM) model. An MM xenograft mouse model with OPM2 cells engrafted in immunocompromised NOD-SCID IL-2Rγ^{null} (NSG) mice was utilized. Accordingly, NSG mice were intravenously injected with OPM2 cells (human MM, 1 million cells) via the tail vein. Two weeks after OPM2 cell injection, freshly prepared BCMA CAR T cells (0.5 million CAR⁺ cells containing an 41BB-CD3 CAR construct) were intravenously administered. A cohort of mice received the BCMA-muIL2 CAR-engager treatment (FIG. 9B). NSG mice (n=5) were injected with OPM2 (human MM) cells followed by BCMA CAR (human) T cell administration according to the schedule. BCMA-muIL2 CAR-E treatment (200 μg) was administered twice per week for two weeks, followed by once per week until the endpoint. After one month or longer, mice were euthanized, and flow cytometric analyses were performed on the harvested organs. These results revealed a significant expansion of CAR T cells in the spleen and bone marrow of the BCMA-muIL2 CAR-engager-treated group, demonstrating over a 100-fold selective expansion of CAR T cells compared to non-treated animals that only received CAR T cells in the spleen (FIG. 9C, left panel) and bone marrow (FIG. 9C, right panel). These experiments were replicated multiple times with similar outcomes (FIG. 9D and FIGS. 15A-15C; n=12 for CAR T cells only, n=22 for CAR T cells plus CAR-engager treatment).

BCMA CAR T cells were detected by co-staining with an anti-human CD45 antibody and Alexa647-labeled BCMA antigen. Similar results were obtained in repeated experiments. Additional control cohorts received VHH-muIL2 treatment with the same dose and schedule as BCMA-muIL2. FIG. 9D shows pooled data from these experiments. Data were analyzed by group mean comparisons using one-way ANOVA and subsequent Tukey post-hoc analysis. Individual flow graphs for the pooled data are shown in FIGS. 15A-15C. Error bars represent mean with standard deviation.

The control group that received CAR T cells plus VHH-muIL2 treatment (n=7) did not exhibit significant expansion or persistence of CAR T cells compared to the control group that received only CAR T cells without treatment. These results show that the BCMA-muIL2 CAR-engager can expand CAR T cells in vivo. Further analysis revealed that the BCMA-muIL2 treatment had a more pronounced effect on CD8⁺ CAR T cells specifically, resulting in an unexpected significant increase in their proportion from the initial ~30% to ~70% of the total CD4⁺ and CD8⁺ CAR T cell population (FIG. 9E). The cohorts receiving only CAR T cells or CAR T cells with the VHH-muIL2 control treatment did not yield a sufficient number of persisting CAR cells for a similar analysis. Data were analyzed by group mean comparisons using one-way ANOVA and subsequent Tukey post-hoc analysis. Error bars represent mean with standard deviation.

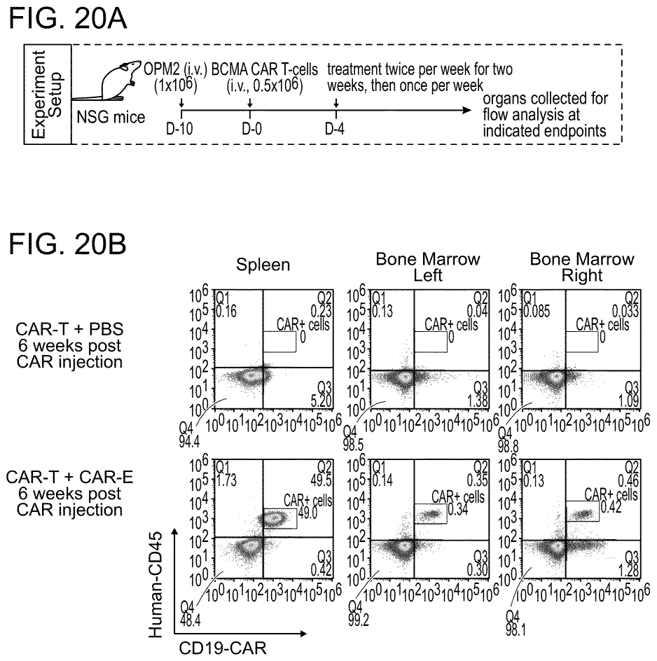

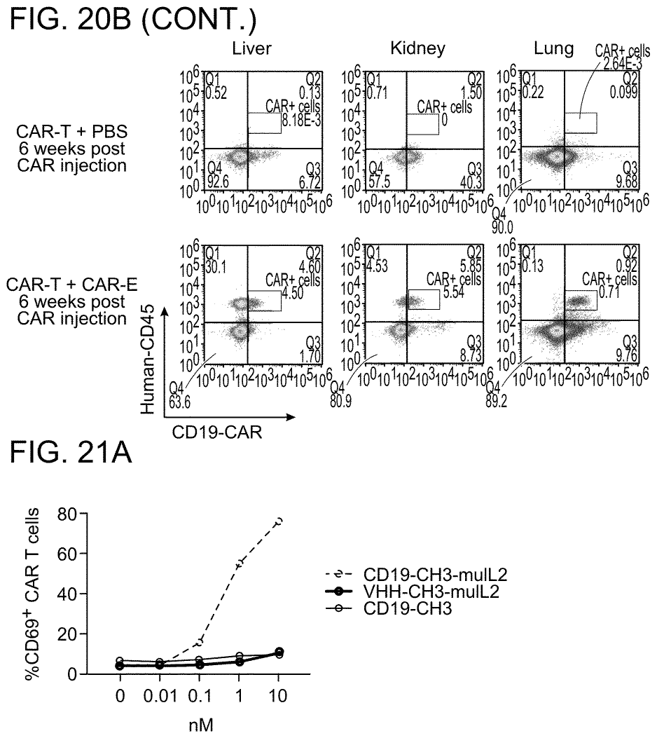

The BCMA CAR-engager enhances CAR T-cell trafficking in an MM model. An MM xenograft mouse model with OPM2 cells engrafted in immunocompromised NSG mice was utilized. Accordingly, NSG mice were intravenously injected with OPM2 cells (human MM, 1 million cells) via the tail vein. Ten days after OPM2 cell injection, freshly prepared BCMA CAR T cells (0.5 million CAR⁺ cells containing a 41BB-CD3ζ CAR construct) were intravenously administered. A cohort of mice received the BCMA-muIL2 CAR-engager treatment (FIG. 20A). NSG mice (n=5) were injected with OPM2 (human MM) cells followed by BCMA CAR (human) T cell administration according to the schedule. BCMA-muIL2 CAR-E treatment (200 μg) was administered twice per week for two weeks, followed by once per week until the endpoint. After one month or longer, mice were euthanized, and flow cytometric analyses were performed on the harvested organs. These results (FIG. 20A) revealed a significant CAR T cell trafficking to the spleen, bone marrow, liver, kidney, and lung of the BCMA-muIL2 CAR-engager-treated group, demonstrating the presence and persistence of CAR T cells in all major organs tested as compared to non-treated animals that only received CAR T cells (FIG. 20B).

### Example 8: BCMA CAR-Engager Treatment Enables CAR T Therapy with Low-Dose of CAR T Cells

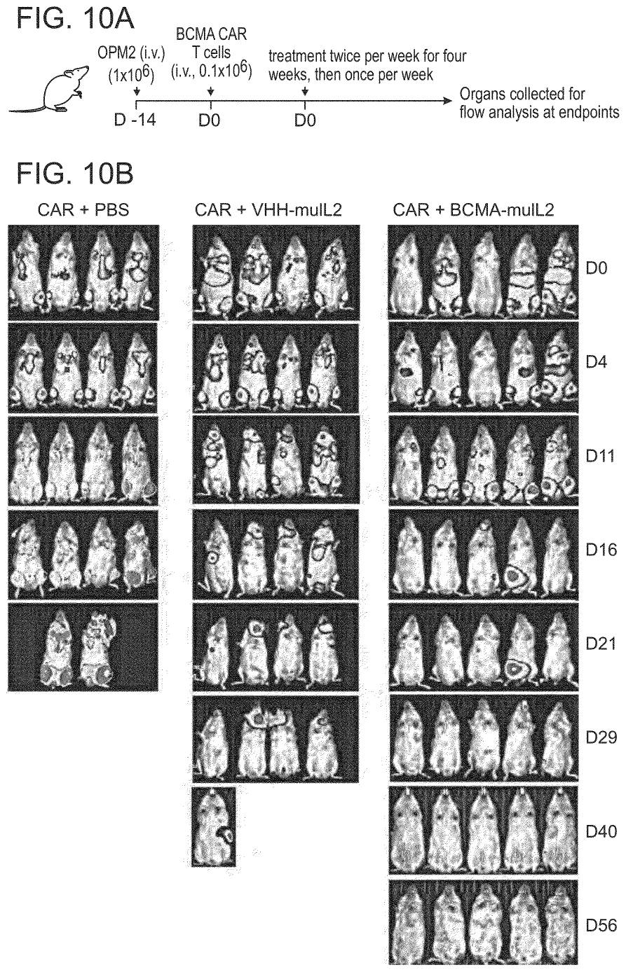

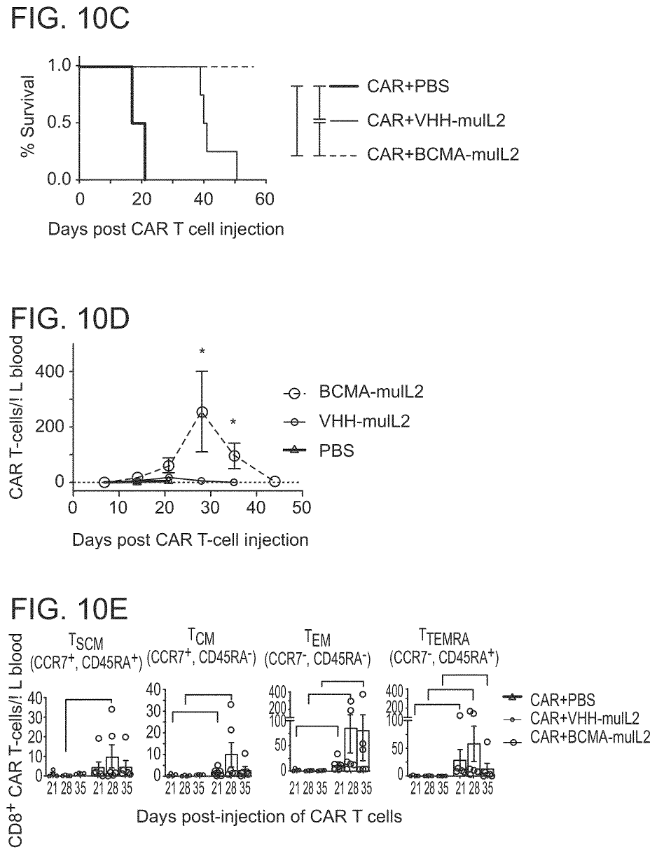

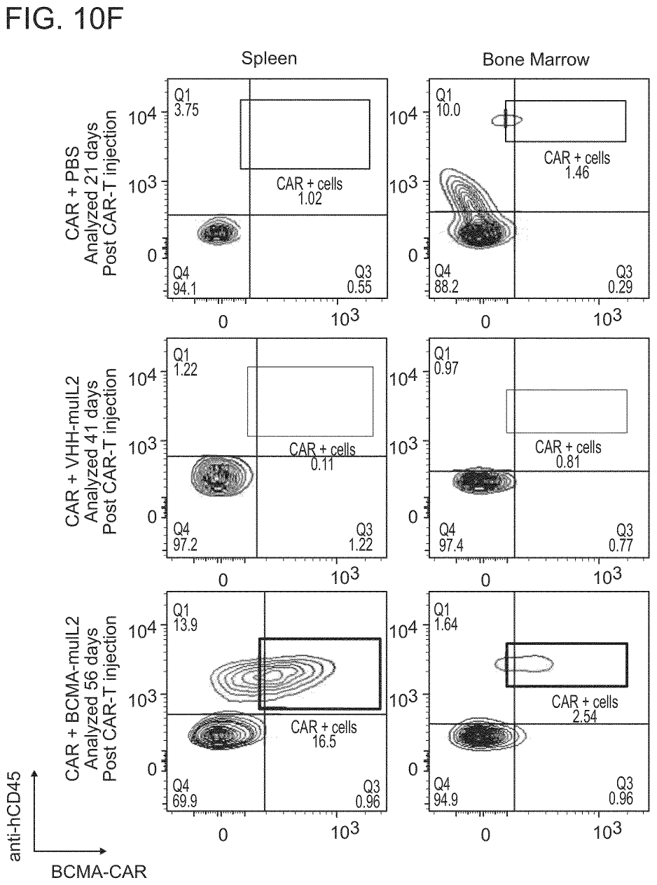

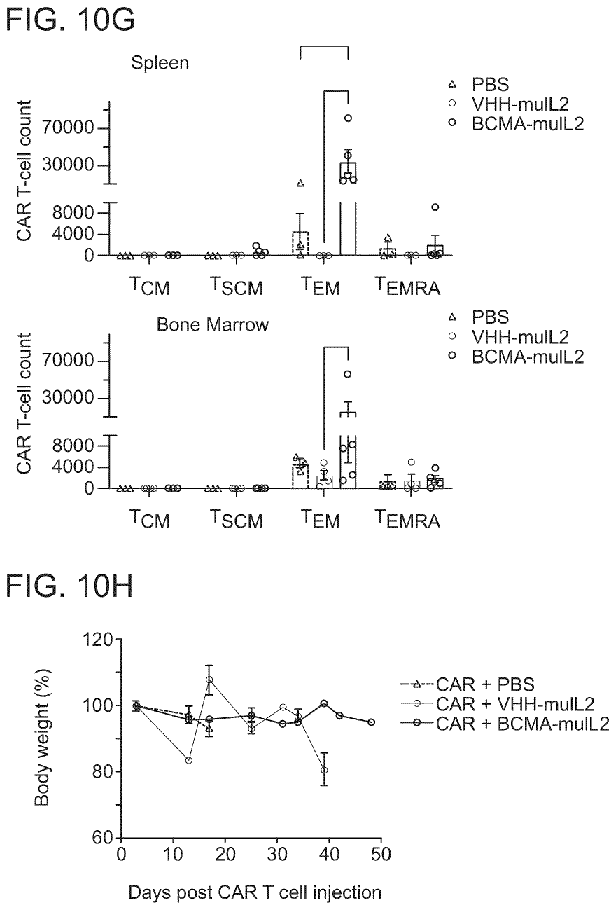

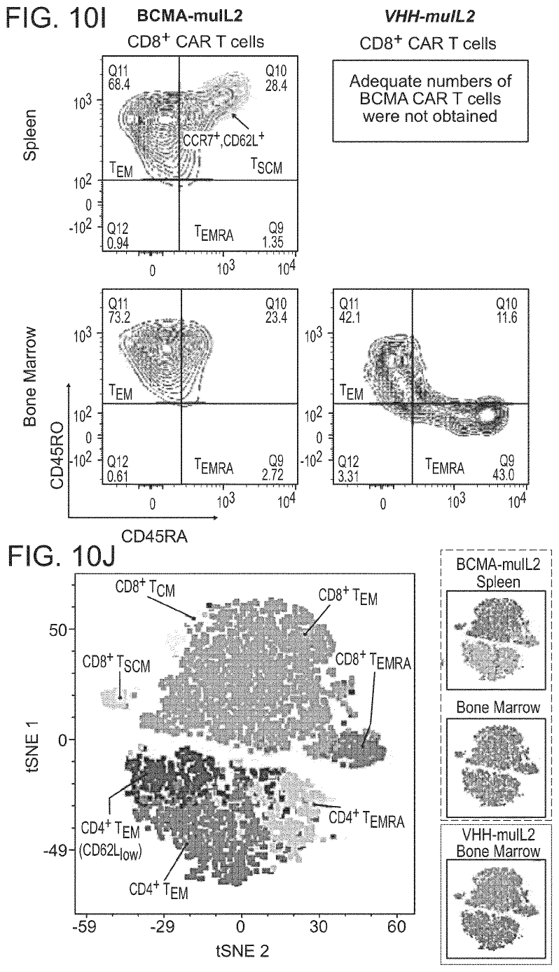

To further demonstrate the effectiveness of CAR-engager treatment and clearance of tumor cells by CAR T cells, a similar protocol as described above was conducted. However, in this study, a lower dose of only 100,000 CAR T cells was utilized (FIG. 10A). All mice treated with BCMA-muIL2 CAR-engager achieved complete tumor clearance (5/5), whereas not a single control mouse receiving either only CAR T cells (n=4) or CAR T cells combined with VHH-muIL2 treatment (n=4) were able to eliminate the tumors (FIGS. 10B-10C).

Analysis of blood samples collected at various time points revealed a substantial expansion of CAR T cells in the circulation following CAR-engager treatment, with the peak expansion observed at week 4 (FIG. 10D). Flow cytometric analyses of blood samples revealed robust expansion of CAR T cells in the treatment group compared to PBS or VHH-muIL2 cohorts. In contrast, the VHH-muIL2 treatment, despite slightly enhancing the initial response, did not induce a significant expansion of CAR T cells. Data was analyzed with two-way ANOVA for day 7, 14 and 21. Once all mice in PBS cohort were euthanized, BCMA-muIL2 and VHH-muIL2 comparisons were performed with multiple Mann-Whitney tests on days 28 and 35. Error bars represent mean with S.E.M. This expansion correlated to the levels of IFN-7 detected in the circulation (FIGS. 17A-17C). Additionally, the treatment facilitated the generation of memory CAR T cells, demonstrating long-lasting effects (FIG. 10E and FIGS. 17A-17C). *P<0.05, **P<0.01. Error bars represent mean with S.E.M.

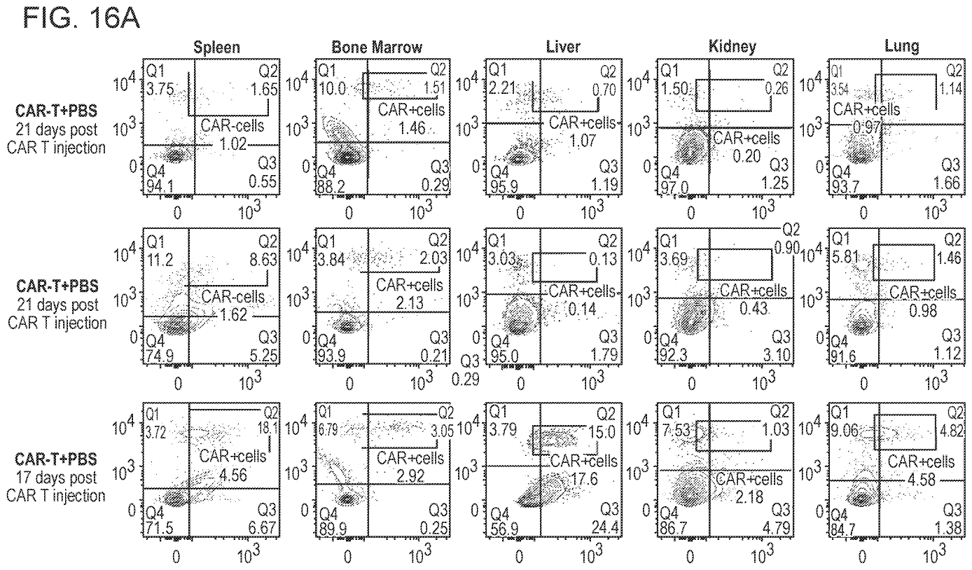

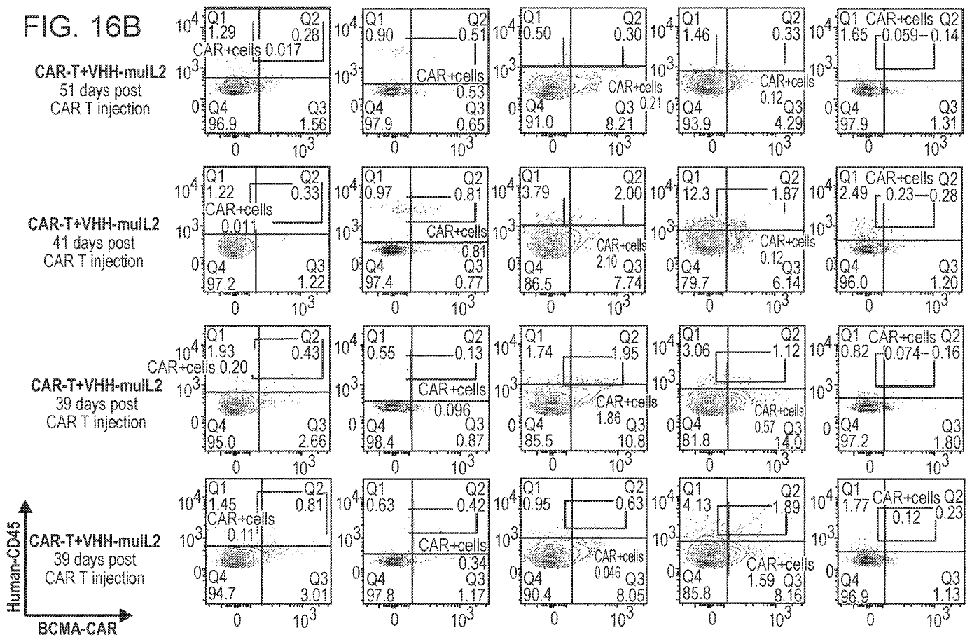

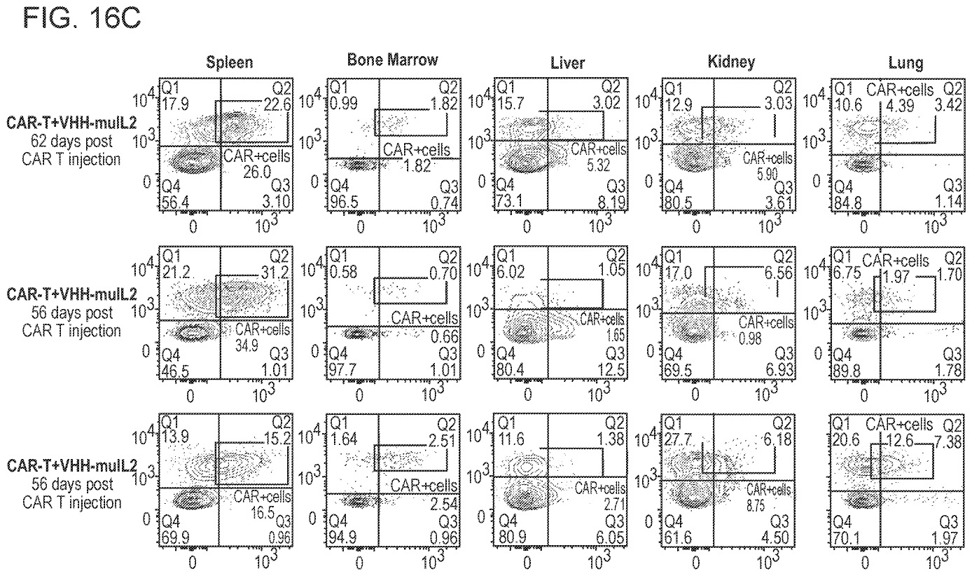

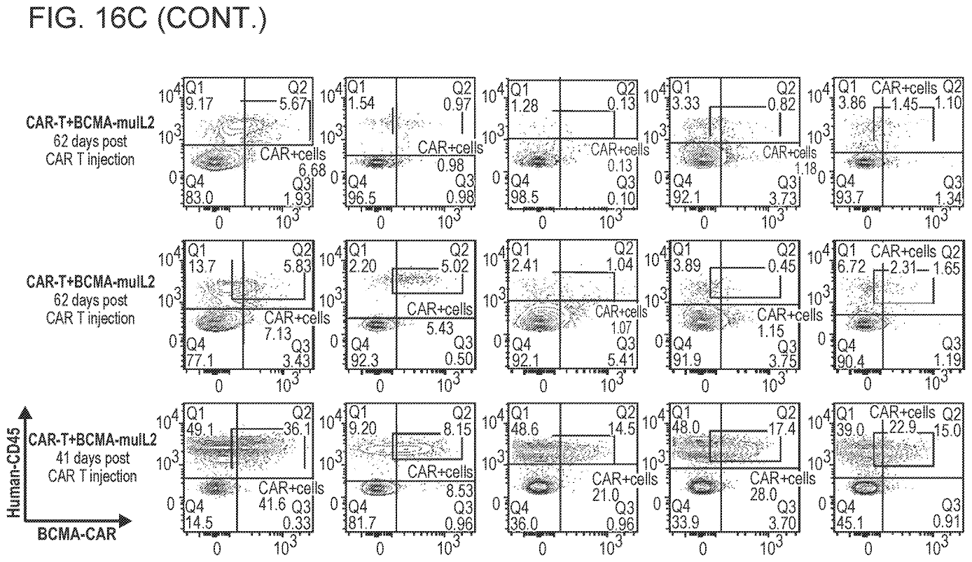

The mice treated with CAR-engager exhibited no signs of toxicity based on clinical observations and weight measurements (FIG. 10H). Subsequent analysis conducted two months after CAR T cell injection demonstrated a substantial presence of CAR T cells, including memory CART cells, in the CAR-engager treated mice (FIGS. 10F-10J, FIGS. 16A-16C, and FIGS. 17A-17C). In the CAR+PBS group, CAR T cells were detected in the spleen; however, these mice succumbed to tumor growth at around 20 days post-CAR T cell injection. The BCMA-muIL2 treatment had also increased the presence of CAR T cells in bone marrow compared to PBS or VHH-muIL2 cohorts, but the difference was less significant than spleen. Data were analyzed by two-way ANOVA with Tukey's multiple comparisons test. *P<0.05, ***P<0.001, ****P<0.0001. Individual flow data are shown in FIGS. 16A-16C. Error bars represent mean with S.E.M. Data in FIG. 10I show that Persisting CAR T cells in the BCMA-muIL2 treated mice's spleens exhibited a CCR7⁺CD45RA⁺CD62L⁺ stem-cell memory phenotype, which was absent in the CAR+VHH-muIL2 or CAR+PBS cohorts.

tSNE analysis was based on surface marker expression of CD8a, CD4, CD45, CD45RA, CD45RO, CD62L, CD69, PD-1, HLA-DR, CCR7, and BCMA-CAR and revealed the presence of distinct memory T cell populations. CAR T cells were detected in the bone marrow of the VHH-muIL2-treated group but not the spleen. In the BCMA-muIL2 group, persisting CAR T cells were predominantly CD8 T cells, while the majority of bone marrow CAR T cells in the VHH-muIL2 group were CD4 T cells. Further analyses are shown in FIG. 17A-17C. The Flt-SNE mapping shown in FIG. 17C is of CAR T cells derived from the PBS, BCMA-muIL2 and VHH-muIL2 treated mice as shown in FIG. 10A. The expression of ten immune cell markers (CD45-Pacific Blue, CD8-FITC, CD4-PE Dazzle594, BCMA-CAR (antigen)-AlexaFluor647, CD69-BV421, PD-1-BV605, CD45RA-APC-Cy7, CD45RO-PerCP-Cy5.5, CD62L-PE, CCR7-AlexaFluor700) on splenocytes and bone marrow from 3 PBS mice, 3 BCMA-muIL2 mice and 4 VHH-muIL2 mice were analyzed by flow cytometry. CD45⁺, α-BCMA-CAR⁺ immune cells from the mice were concatenated to form a total of ~9800 (PBS spleen), ~8100 (BCMA-muIL2 spleen), ~7600 (PBS bone marrow), ~14200 (BCMA-muIL2 bone marrow), ~1420 (VHH-muIL2 Bone Marrow). The entire high dimensional dataset was merged to create a single Flt-SNE map for each condition with the signal strength of various phenotypic markers defining specific immune phenotypes expressed with a blue-green-yellow-red continuous color scale. FltSNE was conducted with the following parameters: max iterations: 1000, theta: 0.5, learning rate: 200, perplexity: 20. There were inadequate numbers of CAR T cells in VHH-muIL2 cohort spleen to conduct Flt-SNE. To enhance visibility, the dots representing the VHH-muIL2 bone marrow samples were enlarged, as fewer cells were detectable in these mice. In the BCMA-muIL2 treated mice, the majority of CAR⁺ cells were CD8⁺ cells, while in the VHH-muIL2 samples, CD4⁺ cells constituted the majority of CAR⁺ cells. Notably, CAR⁺ cells in the CAR+PBS cohort exhibited low or no expression of CD45RA, CD45RO, or CD62L, whereas the BCMA-muIL2 treated mice showed a CAR⁺ population with elevated expression levels of these memory markers.

Thus, the treatment not only facilitates robust proliferation and eradication of tumor cells using low doses of CAR T cells but also promotes the development of long-lasting memory cells, demonstrating the efficacy of BCMA-muIL2 CAR-engager treatment in enhancing the clearance of tumor cells by CAR T cells, and generation of long-lasting memory cells.

### Example 9: Persisting CAR T Cells Treated with CAR-Engager Treatment Remain Functional Three Months Post Infusion

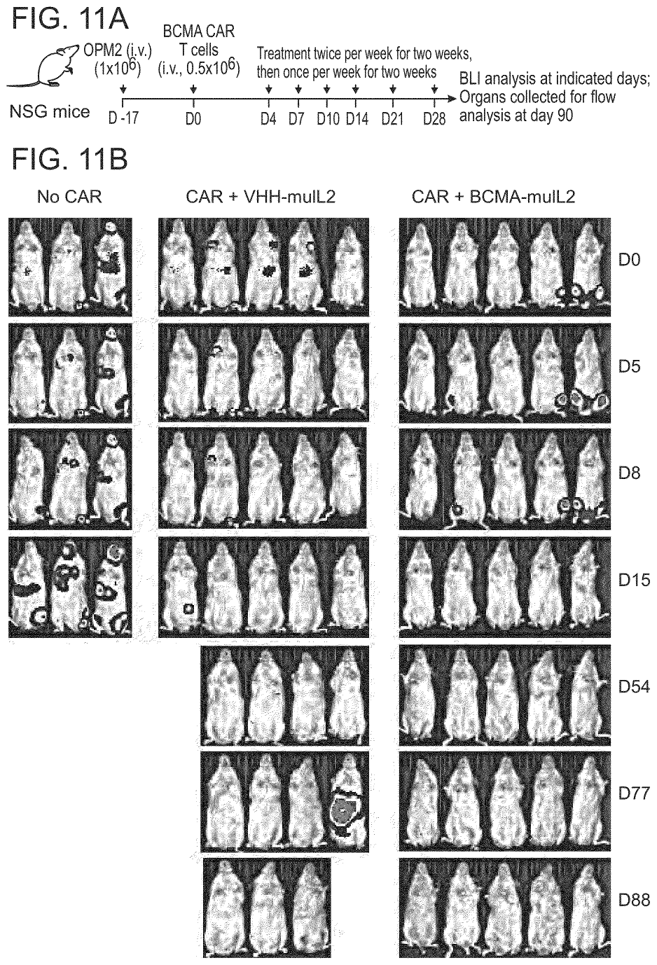

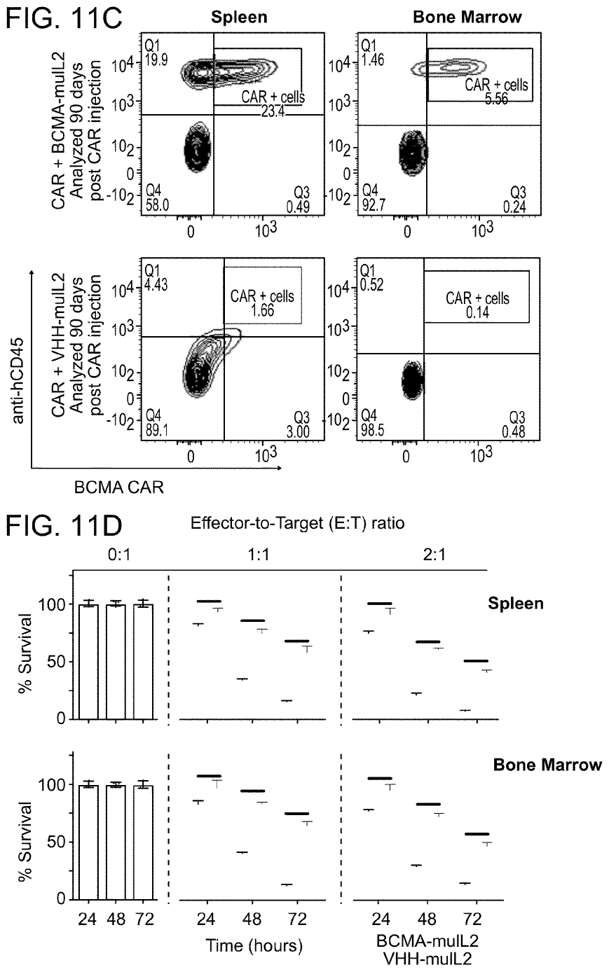

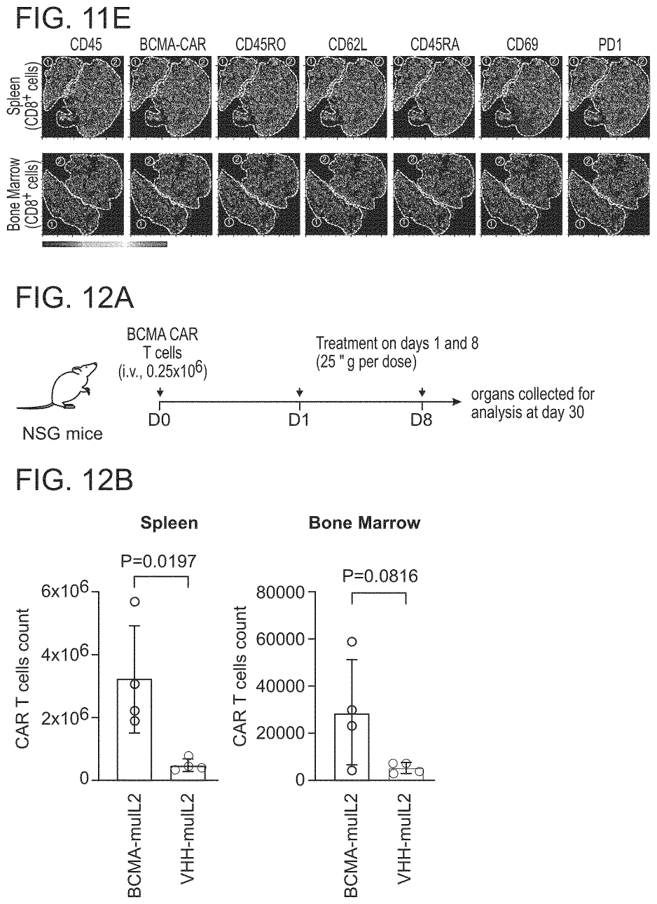

Mice received 1 million OPM2 cells followed by 0.5 million BCMA CAR T cells (FIG. 11A). One group of mice received CAR-engager treatment, administered twice per week for two weeks, followed by once per week for an additional two weeks (6 doses, 200 μg per dose on days 4, 10, 14, 17, 21, and 28; n=5). The control group received VHH-muIL2 treatment at the same dosage and schedule (n=5), while an additional control cohort received only tumor cells (n=3). All mice receiving CAR T cells exhibited an initial response compared to control mice without CAR T cells (FIG. 11B). All CAR-engager treated mice (5 out of 5) and 3 out of 5 mice in the VHH-muIL2 group survived for over three months, which encompassed the duration of the experiment. One VHH-muIL2 mouse died in about a month, and a second mouse succumbed to cancer cell relapse with liver metastasis (FIG. 11B, day 77). The surviving mice were euthanized three months post-injection of CAR T cells, and the splenocytes and bone marrow cells were analyzed to assess the presence of CAR T cells. Remarkably, CAR-engager-treated mice exhibited a significant abundance of CAR T cells homing and persisting in the bone marrow and spleen compared to mice receiving CAR T cells with VHH-muIL2 treatment (FIG. 11C). Given the two-month period of no treatment before the mice were sacrificed, these results further suggest that the treatment facilitated the generation of memory cells among CAR T cells.

To demonstrate the functionality of the persisting CAR T cells in CAR-engager-treated mice, an in vitro killing assay was performed using the BCMA CAR T cells harvested from bone marrow and spleen. Bone marrow and splenocytes were analyzed via flow cytometry to detect and quantify CAR-expressing T cells and the bone marrow cells or splenocytes were co-incubated with OPM2 target cells at various E:T ratios (1:1 and 2:1) based on CAR-expressing cells. Survival was determined 24, 48, and 72 hours later using flow cytometric analysis. The three-month-old CAR T cells demonstrated efficient killing of tumor cells and long-term functionality (FIG. 11D). Only one of the VHH-muIL2 treated mice exhibited sufficient CAR T cells to perform a similar killing assay, and while it exhibited tumor cell killing, the efficiency was lower than that observed in CAR-engager treated CAR T cells (FIG. 11D) (error bars represent mean with standard deviation). Therefore, CAR-engager treatment robustly expands and drives the persistence of CAR T cells while maintaining their killing potential.

To further characterize the phenotype of these persistent CAR T cells, flow cytometric analyses were conducted to evaluate the expression of a series of T cell markers (CD45, BCMA CAR, CD4, CD8, CD62L, CD45RO, CD45RA, CD69, and PD-1). In order to facilitate interpretation, a t-SNE mapping of splenocytes, and bone marrow cells was generated (FIG. 11E). anti-CD45-Pacific Blue, anti-CD8-FITC, anti-CD4-PE Dazzle594, BCMA (antigen)-AlexaFluor647, anti-CD69-BV421, anti-PD-1-BV605, anti-CD45RA-APC-Cy7, anti-CD45RO-PerCP-Cy5.5, anti-CD62L-PE, and CCR7-AlexaFluor700) on splenocytes and bone marrow from the five BCMA-muIL2 treated mice were analyzed by flow cytometry. CD45⁺, CD8⁺, α-BCMA-CAR⁺ cells from the five mice were concatenated to form a total of ~17600 (spleen) and ~10800 (bone marrow) cells. The entire high dimensional dataset (excluding the CD45, CD8, and CD4 parameters) was merged to create a single tSNE map with the signal strength of six phenotypic markers defining specific immune phenotypes expressed with a blue-green-yellow-red continuous color scale. tSNE analysis was performed using 1000 iterations, a perplexity of 30 and a learning rate of 1237 and 756 for spleen and bone marrow respectively. Population labeled as 1 appears to display a memory-like phenotype, expressing higher levels of CD45RO, CD62L and CD45RA. Population labeled as 2 appears to display an effector-like phenotype, expressing low levels of CD45RO, CD62L and CD45RA.

The flow cytometric analyses revealed that the persisting CAR T cells consisted of both CD4⁺ and CD8⁺ populations. The CD8 CAR cells appeared to exhibit two distinct populations: effector cells and CD45RA⁺CD62L⁺ memory cells. The memory population exhibited higher expression levels of BCMA CAR and CD45 (FIG. 11E). Similarly, CD4⁺ CAR T cells demonstrated two populations of effector and memory cells (FIG. 18). An insufficient number of CAR T cells could be detected from the VHH-muIL2 treated mice to perform a similar flow cytometric analysis. Therefore, the CAR-engager treatment leads to generation of long-lasting memory CAR T cells.

FIG. 18 shows t-SNE mapping of CD4⁺ CAR⁺ T cells derived from the five BCMA-muIL2 CAR-E treated mice as shown in FIGS. 11A-11E. The expression of nine immune cell markers (aCD45-Pacific Blue, aCD8-FITC, aCD4-PE Dazzle594, BCMA (antigen)-AlexaFluor647, aCD69-BV421, aPD-1-BV605, aCD45RA-APC-Cy7, aCD45RO-PerCP-Cy5.5, aCD62L-PE) on splenocytes and bone marrow from the five BCMA-muIL2 treated mice were analyzed by flow cytometry. CD45⁺, CD4⁺, α-BCMA-CAR⁺ immune cells from the five mice were concatenated to form a total of ~9000 (spleen) and ~6600 (bone marrow) cells. The entire high dimensional dataset (excluding the CD45, CD8, and CD4 parameters) was merged to create a single t-SNE map with the signal strength of six phenotypic markers defining specific immune phenotypes expressed with a blue-green-yellow-red continuous color scale. tSNE analysis was performed using 1000 iterations, a perplexity of 30 and a learning rate of 630 and 466 for spleen and bone marrow, respectively. The population labeled as “1” appears to display a memory-like phenotype, expressing higher levels of CD45RO, CD62L and CD45RA. The population labeled as “2” appears to display an effector-like phenotype, expressing low levels of CD45RO, CD62L and CD45RA.

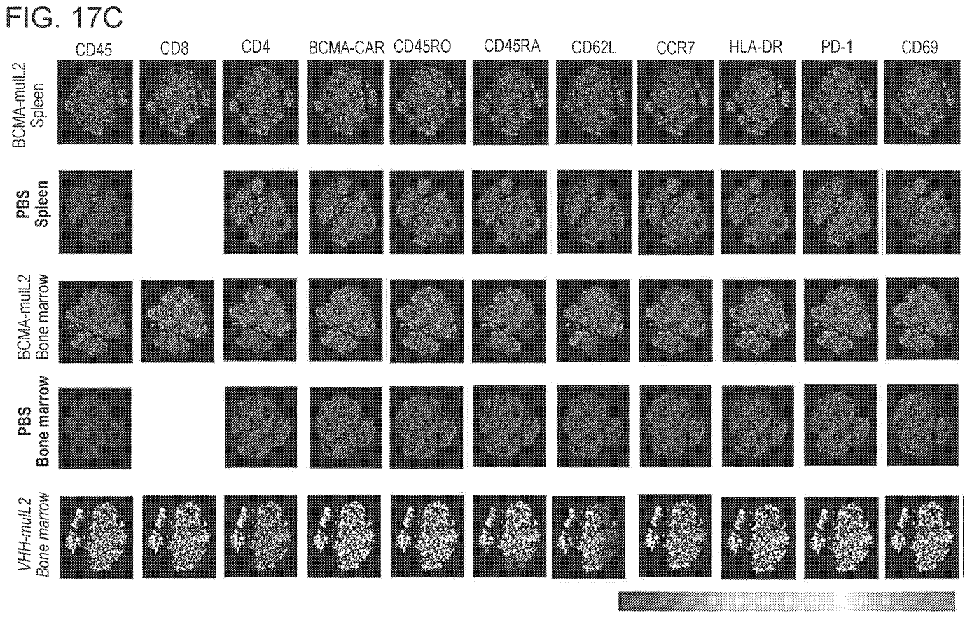

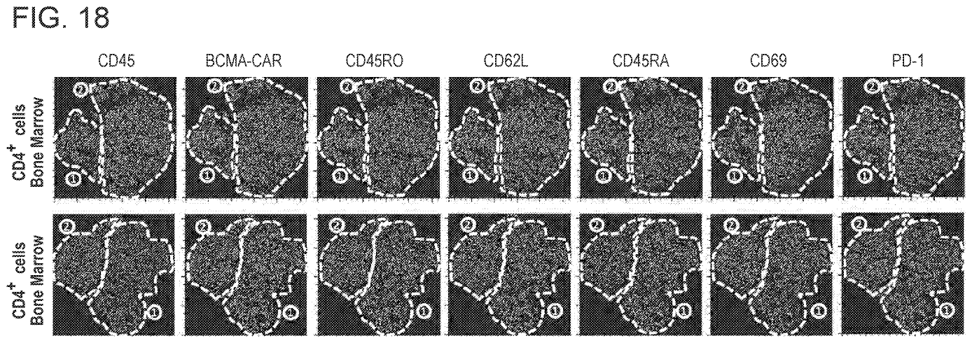

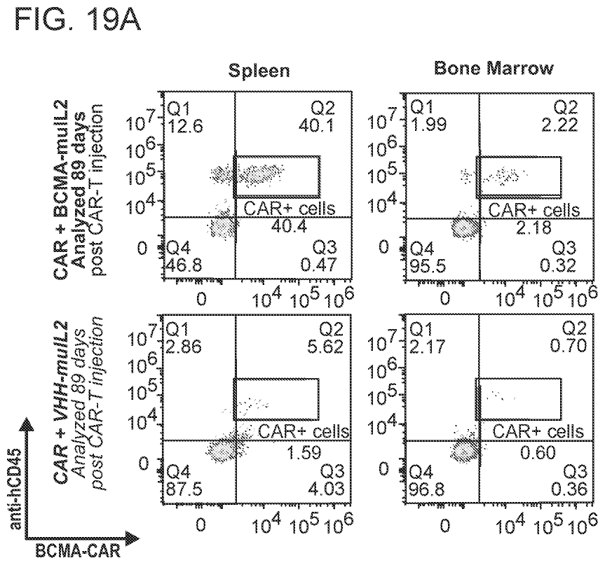

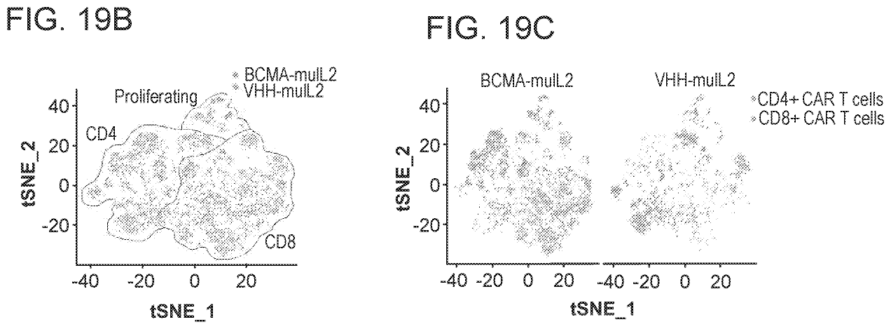

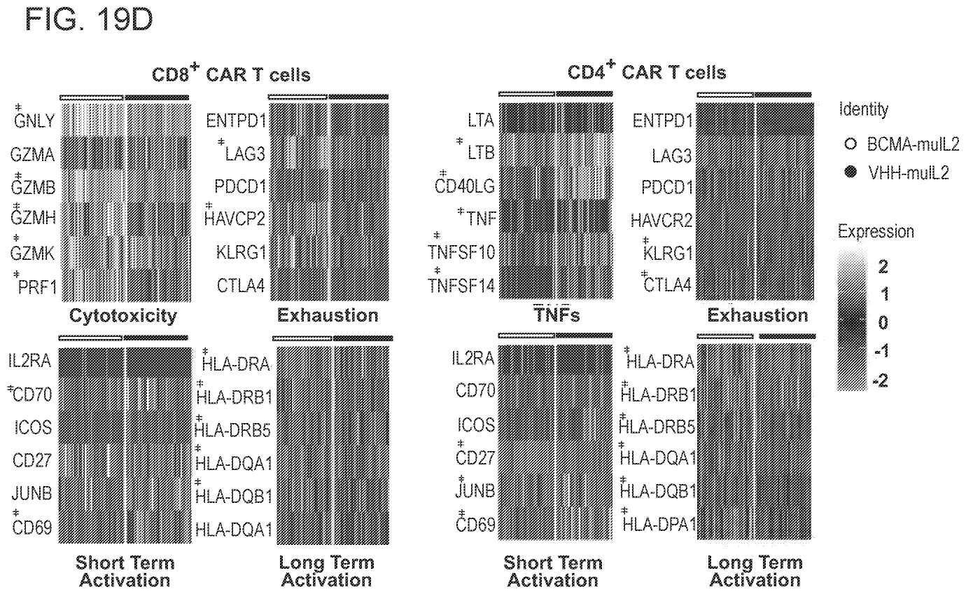

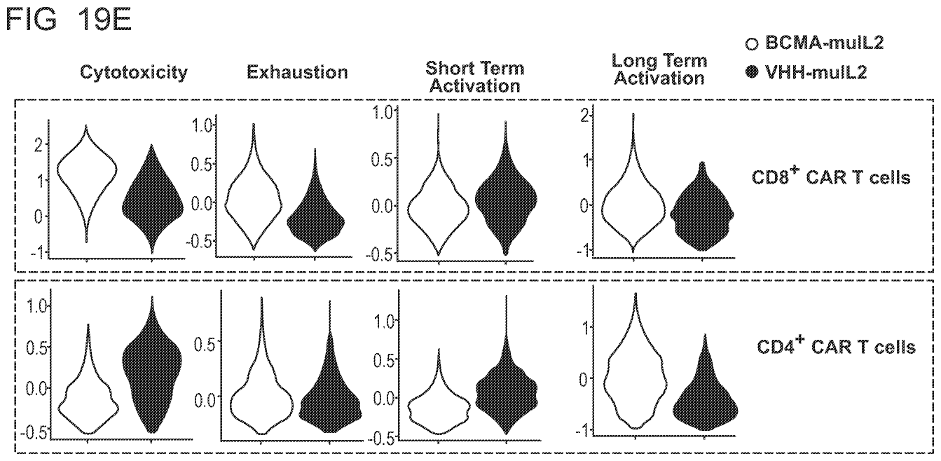

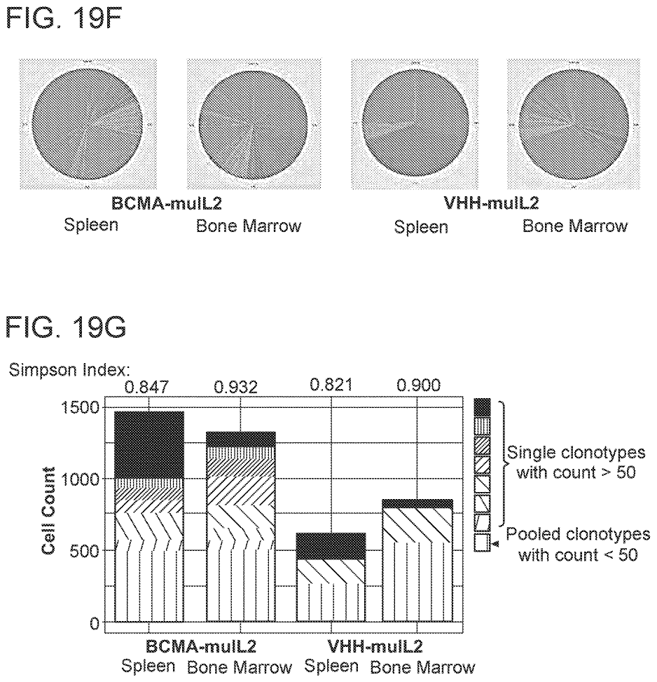

Single-cell RNA-sequencing (scRNAseq) analysis was performed on CAR⁺ T cells isolated from mice treated with either the BCMA-muIL2 or the VHH-muIL2 control. Despite the limited presence of CAR T cells in the VHH-muIL2-treated mice, a sufficient number of cells were obtained from one of the VHH-treated mice for the experiment (FIG. 19A). CAR⁺ cells were sorted after staining with BCMA-AlexaFluor647 and TotalSeq-C hashing antibodies from BCMA-muIL2 or VHH-muIL2 treated mice as shown in the red and green boxes, respectively in FIG. 19A. 5000 CAR⁺ cells from BCMA-muIL2 mice bone marrow and spleen and 2500 CAR⁺ cells from VHH-muIL2 mice bone marrow and spleen were loaded onto the 10× channel. The scRNAseq analysis revealed that the predominant population of persistent CAR T cells in the BCMA-muIL2-treated mouse consisted of CD8⁺ T cells (FIGS. 19B-19C), which exhibited an enrichment of genes associated with an activated T cell state (FIGS. 19D-19E). Heatmaps in FIG. 19D show significantly differentially expressed genes between CAR-engager treatment and VHH conditions in CD8 and CD4 CAR T cells, split among different relevant conditions. Genes marked with an * are the significantly differentially expressed genes between BCMA-muIL2 and VHH-muIL2 treated mice in the subset of interest. This was evidenced by elevated expression levels of granzyme family genes, other cytotoxicity-associated genes, and MHC class II genes. No significant differences in activation markers were observed between CAR T cells obtained from the BCMA-muIL2 or VHH-muIL2 treated mice, as the mice had already cleared the tumors over 60 days prior. The BCMA-muIL2 treatment did not induce upregulation of exhaustion markers, showing the treatment did not induce exhaustion in the persisting CAR T cells.

Next, the diversity of T-cell receptor (TCR) clonotypes was evaluated in the BCMA-muIL2 and VHH-muIL2 treated mice (FIGS. 19F-19G). Both groups displayed similar diversity in clonotypes present, showing that the BCMA-muIL2 CAR-engager treatment could effectively facilitate the generation of a diverse TCR repertoire in persisting CAR T cells, as opposed to promoting the dominance of a restricted set of TCR clones. Pie plots in FIG. 19F show the diversity of TCR clonotypes, with each slice of the pie chart representing the proportion of a different TCR clonotype present; colors were randomly assigned to different clonotypes. The clonotype diversity within each sample's total cell count was visualized with a stacked bar plot (FIG. 19G), where similar clonotypes with counts below 50 were combined. To evaluate the diversity within each sample, the Simpson index was calculated, with higher values indicating greater diversity. Overall, the results showed that the BCMA CAR-engager could not only help CAR T cells to fully clear tumor cells, but also robustly induce generation of long-lasting and functional memory CAR T cells.

### Example 10: CAR-Engager Expands CAR T Cells in the Absence of Tumor Antigens

CAR T cell expansion typically occurs following infusion in patients, with peak expansion observed around 10-14 days post-infusion (Rodriguez-Otero et al., N. Engl. J. Med. 388(11):1002-1014 (2023)).

Eradication of minimal residual disease (MRD) facilitates long-lasting and complete responses. However, the limited presence of the corresponding antigen associated with MRD may not adequately support the proliferation and efficacy of conventional CAR T cells. To demonstrate efficacy in the absence of tumor antigen, NSG mice were solely injected with 0.25 million BCMA CAR T cells in the absence of tumor cells. These mice received two 25 g doses of BCMA-muIL2 on days 1 and 8 post-injection of CAR T cells. The control group received VHH-muIL2 treatment (n=4 for each group). On day 30, the mice were euthanized, and their spleen and bone marrow were assessed for the presence of CAR T cells. The BCMA-muIL2 treated mice exhibited higher numbers of CAR⁺ T cells in the spleen (approximately 6.8-fold higher) and bone marrow (approximately 5.5-fold higher), showing that BCMA CAR-engager expanded CAR T cells in vivo, even without the presence of tumor cells (FIGS. 12A-12B); error bars represent mean with standard deviation. Overall, these findings demonstrate that the CAR-engager can expand CAR T cells, even in the absence of tumor antigen. Additionally, the effectiveness of the treatment was evident even at lower doses and frequencies.

### Example 11: CD19 CAR-E does not Inhibit Killing Efficacy of CD19 CAR T-Cells

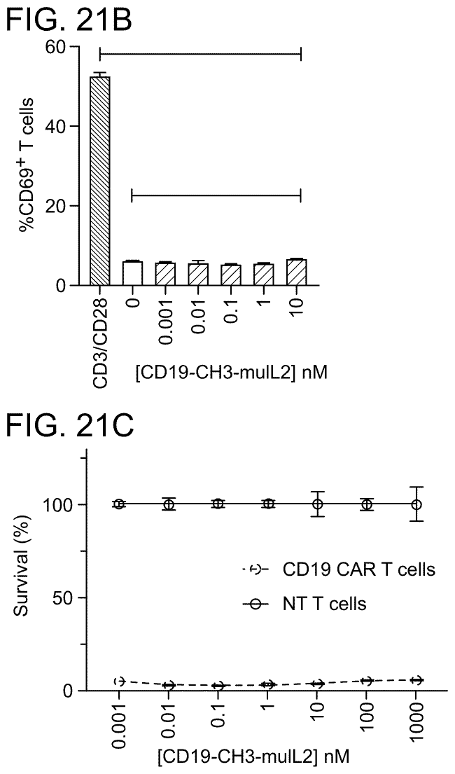

CAR-engagers that bind BCMA were observed to bind but not inhibit killing efficacy of BCMA CAR T cells (FIGS. 2D-2H). To investigate the impact of CAR-E containing other cancer antigens on antigen-specific CAR T cells, a killing assay was conducted using CD19 CAR T cells and patient-derived CD19⁺ leukemia cells in the presence of varying concentrations of the CD19 CAR-engager. Remarkably, the results demonstrated no inhibition of killing even at the highest tested concentration (1000 nM of the CAR-engager) (FIG. 21C). Nalm6 cells were co-incubated with CD19 CAR T cells (filled) or non-transduced T cells (open) (E:T ratio 1:1, 30,000 cells of each) in the presence of varying concentrations of the CD19-muIL2 CAR-E treatment. Live (PI⁻) Nalm6 cells were counted 48 hours later, with an N of 3 for each of the experiments. The experimental findings disclosed herein with CD19 are consistent with the findings in BCMA cancer models and BCMA CAR-E, above.

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A chimeric antigen receptor (CAR)-engager protein, comprising:
a first moiety comprising an ectodomain of an antigen present on a cancer cell connected to a second moiety comprising a first immune cell effector domain; or
a first moiety comprising an ectodomain of an antigen connected to a second moiety comprising a first immune cell effector domain.

2. The CAR-engager of claim 1, wherein the ectodomain is derived from AFP, AXL, B4GALNT1, B-cell maturation antigen (BCMA), CA9, CD5, CD7, CD19, CD20, CD22, CD23, CD33, CD34, CD38, CD44, CD52, CD70, CD80, CD86, CD123, CD133, CD174, CD274, CD276, CDS, Cancer/Testis Antigen 1B (CTAG1B), carcinoembryonic antigen (CEA), CLEC12A, claudin 18.2 (CLDN 18.2), CSPG4, DLL3, EGFR, EPCAM, EPHA2, ERBB2, FAP, FOLH1, FOLR1, GD2, GPC3, GPRC5D, GPNMB, HER2, HPV E7, ILIRAP, IL3RA, IL13Rα2, KDR, KIT, KLRK1, L1CAM, MAGEA1, MAGEA4, MET, MME, MSLN, MUC1, MUC16, MS4A1, NCAM1, PD-1, PMEL, PROM1, PSCA, ROR1, ROR2, SDC1, SLAM7, TEM1, TROP2, TNF Receptor Superfamily Member (TNFRSF) 8, TNFRSF10B, TNFRSF13C, TNFRSF17, ULBP1, or ULBP2.

3. The CAR-engager of claim 2, wherein the ectodomain is derived from BCMA.

4. The CAR-engager of claim 3, wherein the ectodomain comprises the amino acid sequence SEQ ID NO: 1 or SEQ ID NO: 2.

5. (canceled)

6. The CAR-engager of claim 2, wherein the ectodomain is derived from CD19.

7. The CAR-engager of claim 6, wherein the ectodomain comprises an amino acid sequence SEQ ID NO: 3 or that has at least about 85% sequence identity with amino acid sequence SEQ ID NO: 3, amino acid sequence SEQ ID NO: 4, or amino acid sequence SEQ ID NO: 5.

8.-9. (canceled)

10. The CAR-engager of claim 2, wherein the ectodomain is derived from CD20, SLAMF7, PD-1, KIT, CD38, or CD22.

11. The CAR-engager of claim 10, wherein the ectodomain is derived from CD20 and comprises the amino acid sequence SEQ ID NO: 6;
or wherein the ectodomain is derived from SLAMF7 and comprises the amino acid sequence SEQ ID NO: 10; or
wherein the ectodomain is derived from PD-1 and comprises the amino acid sequence SEQ ID NO: 11; or
wherein the ectodomain is derived from KIT and comprises the amino acid sequence SEQ ID NO: 12; or
wherein the ectodomain is derived from CD38 and comprises the amino acid sequence SEQ ID NO: 14; or
wherein the ectodomain is derived from CD22 and comprises the amino acid sequence SEQ ID NO: 7 or SEQ ID NO: 107.

12.-22. (canceled)

23. The CAR-engager of claim 1, further comprising a first linker that connects the first and second moieties.

24. The CAR-engager of claim 23, further comprising a dimerization domain disposed between the first linker and the second moiety comprising the first immune cell effector domain; or
further comprising a dimerization domain disposed between the first linker and the second moiety comprising the first immune cell effector domain and a second linker that connects the dimerization domain and the second moiety, wherein the first and second linkers may be the same or different.

25. (canceled)

26. The CAR-engager of claim 24, wherein the first linker and the second linker are flexible; or
wherein the first linker and the second linker are flexible and are derived from the hinge region of CD3ζ, CD4, CD8α, CD28, IgG1, IgG2, or IgG4.

27. (canceled)

28. The CAR-engager of claim 26, wherein the first linker and/or the second linker comprises the amino acid sequence GGGX, GGGGX (SEQ ID NO: 69), GSSGSX (SEQ ID NO: 70), GGGGS (SEQ ID NO: 71), or GSPRG (SEQ ID NO: 72), wherein X is either C or S; or
wherein the first linker has the amino acid sequence of SEQ ID NO: 71, or SEQ ID NO: 72 and the second linker has the amino acid sequence of SEQ ID NO: 76.

29. (canceled)

30. The CAR-engager of claim 24, wherein the dimerization domain is derived from IgA, IgD, IgG, IgM, or IgE.

31. The CAR-engager of claim 30, wherein the dimerization domain comprises an IgG1 constant heavy (CH) 3 domain.

32. The CAR-engager of claim 30, wherein the dimerization domain further comprises an IgG CH2 domain and an IgG CH3 domain.

33. The CAR-engager of claim 1, wherein the first immune cell effector domain comprises a cytokine or immune cell-activating moiety; and/or
wherein the second moiety further comprises a plurality of immune cell effector domains.

34. The CAR-engager of claim 33, wherein the first immune cell effector domain is derived from CD30L, CD40, CD48, CD58, CD70, CD80, CD86, CD112, GITRL, HVEM, OX40L, SEMAA, SLAM, TIM4, interleukin-2 (IL-2), IL-7, IL-9, IL-10, IL-15, IL-18, IL-21, IL-27, 4-1BBL, or an immune cell-activating variant thereof.

35. (canceled)

36. The CAR-engager of claim 33, wherein the second moiety further comprises a second immune cell effector domain, wherein the first and second immune cell effector domains may be the same or different.

37. The CAR-engager of claim 36, wherein the second immune cell effector domain comprises a weak affinity variant of IL-2 which has the amino acid sequence SEQ ID NO: 19 or wherein the first immune cell effector domain and the second immune cell effector domain each has the amino acid sequence of SEQ ID NO: 20; or
wherein the first immune cell effector domain and the second immune cell effector domain each has the amino acid sequence of SEQ ID NO: 102 with a H16A substitution or a F42A substitution; or
wherein the first, the second, or each of the first and the second immune cell effector domains comprise the amino acid sequence of SEQ ID NO: 26.

38.-40. (canceled)

41. The CAR-engager of claim 36, wherein the first or the second immune cell effector domain comprises the amino acid sequence SEQ ID NO: 26; and/or
wherein each of the first and the second immune cell effector domains comprises the amino acid sequence of SEQ ID NO: 26.

42. (canceled)

43. The CAR-engager of claim 35, wherein the first immune cell effector domain comprises 4-1BBL or an immune cell-activating variant thereof.

44. The CAR-engager of claim 43, wherein the first immune cell effector domain comprises the amino acid sequence SEQ ID NO: 27.

45. The CAR-engager of claim 35, wherein the second moiety further comprises a third immune cell effector domain, wherein any two or more of the first, second, and third immune cell effector domains may be the same or different.

46. The CAR-engager of claim 45, wherein each of the first, the second, and the third immune cell effector domains comprises the amino acid sequence of SEQ ID NO: 27.

47. The CAR-engager of claim 33, wherein the first cell effector domain is a single-chain variable antibody fragment (scFv) that binds and activates immune cells.

48. The CAR-engager of claim 47, wherein the first cell effector domain binds 4-1BB, CD2, CD27, CD28, CD30, CD40L, CD226, CTLA4, GITR, IL-2R, LIGHT, OX40, PD-1, TIM2, SLAM, or TIM1.

49. The CAR-engager of claim 48, wherein the first cell effector domain comprises a scFv that binds CTLA-4 and comprises a VL domain with an amino acid sequence of SEQ ID NO: 36 and a VH domain with an amino acid sequence of SEQ ID NO: 37.

50. (canceled)

51. The CAR-engager of claim 48, wherein the first cell effector domain binds OX40 and comprises a VL domain with an amino acid sequence of SEQ ID NO: 42 and a VH domain with an amino acid sequence of SEQ ID NO: 43.

52. (canceled)

53. The CAR-engager of claim 48, wherein the first cell effector domain binds PD-1 and comprises a VL domain with an amino acid sequence of SEQ ID NO: 52 and a VH domain with an amino acid sequence of SEQ ID NO: 53.

54. (canceled)

55. The CAR-engager of claim 1, wherein the first immune cell effector domain further comprises an immune cell-inhibiting moiety.

56. The CAR-engager of claim 55, wherein the first immune cell effector domain is derived from CD80, CD86, CD112, CD155, CD276 (B7-H3), Ceacam-1, FGL1, galectin-3, HLA-E, HVEM, PD-L1, PD-L2, VISTA, or VTCN1 (B7-H4).

57. The CAR-engager of claim 56, wherein the first immune cell effector domain is derived from PD-L1 and has an amino acid sequence of SEQ ID NO: 64; or
wherein the first immune cell effector domain is derived from CD80 and has an amino acid sequence of SEQ ID NO: 54; or
wherein the first immune cell effector domain is derived from CD276 and has an amino acid sequence of SEQ ID NO: 57; or
wherein the first immune cell effector domain is derived from VTCN1 and has an amino acid sequence of SEQ ID NO: 66.

58.-64. (canceled)

65. The CAR-engager of claim 1, wherein the CAR-engager is in the form of a fusion protein and the first and the second moieties are connected by peptide bonds; or
wherein the first moiety comprising the ectodomain is connected to the second moiety comprising the first immune cell effector domain, or the first linker by an azide-alkyne connection, an oxime or hydrazine connection, a tetrazine-transcyclooctene connection, an azide-nitrone connection, a thiol-alkene connection, an alkene-tetrazole connection, an alkene-tetrazine connection, an alkene-azide connection, a conjugated diene-alkene connection, or an isonitrile-tetrazine connection.

66. (canceled)

67. The CAR-engager of claim 24, which is in the form of a homodimer comprising two of the CAR-engagers.

68. A heterodimeric CAR-engager comprising:
a first moiety comprising an ectodomain of an antigen present on a cancer cell connected to first dimerization domain; and
a second moiety comprising a first immune cell effector domain connected to a second dimerization domain;
wherein the first and the second dimerization domains dimerize to form a heterodimer.

69. The CAR-engager of claim 68, wherein the first dimerization domain comprises a protuberance, the second dimerization domain comprises a cavity sterically compensatory to the protuberance, and wherein the protuberance is positionable within the cavity.

70. A nucleic acid encoding
the CAR-engager of claim 65; or
an ectodomain of an antigen present on a cancer cell fused to a first dimerization domain; or
an immune cell effector domain fused to a second dimerization domain.

71.-73. (canceled)

74. A vector comprising the nucleic acid of claim 70.

75. A cell comprising the vector of claim 74; which is a mammalian cell or a bacterial cell.

76.-77. (canceled)

78. A pharmaceutical composition comprising the CAR-engager of claim 1 and a pharmaceutically acceptable carrier.

79. The pharmaceutical composition of claim 78, further comprising an effective number of immune cells comprising a CAR that comprises an extracellular domain that binds the ectodomain of the CAR-engager, a transmembrane domain and an intracellular domain comprising a stimulatory domain.

80. A method of making a CAR-engager comprising:
culturing the cell of claim 75 in medium under conditions wherein the nucleic acid encoding the CAR-engager is expressed; and
isolating the CAR-engager from the cell and/or medium.

81. A method of treating cancer, comprising:
administering to the subject an effective amount of the CAR-engager of claim 1,
wherein prior to, substantially contemporaneous with, or subsequent to the administering of the CAR-engager, the subject is administered an effective number of immune cells comprising a chimeric antigen receptor (CAR) that comprises an extracellular domain that binds the ectodomain of the CAR-engager, a transmembrane domain, and an intracellular domain comprising a stimulatory domain (CAR immune cells).

82.-83. (canceled)

84. The method of claim 81, wherein the subject is administered the CAR immune cells prior to the administration of the CAR-engager.

85. The method of claim 84, wherein the CAR immune cells are administered at least about 6 months, at least about 9 months, or at least about a year prior to administering the CAR-engager.

86. The method of claim 81, wherein the CAR-engager and the CAR immune cells are co-administered.

87. The method of claim 86, wherein the co-administering comprises administering the CAR immune cells and the CAR-engager substantially simultaneously; or
further comprising contacting the CAR-engager with the CAR immune cells in vitro prior to administering the CAR-engager and immune cells; or
further comprising the steps of:
measuring a concentration of immune cells present within a sample obtained from the subject after administering the immune cells; and
calculating a difference between the concentration of the immune cells administered to the subject and the measured immune cell concentration.

88. The method of claim 86, further comprising contacting the CAR-engager with the CAR immune cells in vitro prior to administering the CAR-engager and immune cells.

89. The method of claim 86, further comprising the steps of:
measuring a concentration of immune cells present within a sample obtained from the subject after administering the immune cells; and
calculating a difference between the concentration of the immune cells administered to the subject and the measured immune cell concentration,
wherein the co-administering comprises administering the CAR-engager when the measured CAR immune cell concentration is less than the administered immune cell concentration.

90. (canceled)

91. The method of claim 81, wherein the CAR immune cells are T cells or NK cells.

92. (canceled)

93. The method of claim 81, wherein the cancer is a hematopoietic cancer.

94. The method of claim 93, wherein the cancer is leukemia, multiple myeloma, or lymphoma.

95. (canceled)

96. The method of claim 81, wherein the cancer is characterized by a solid tumor.

97. The method of claim 96, wherein the cancer is renal cell carcinoma, breast cancer, ovarian cancer, neuroblastoma, glioblastoma, Gliosarcoma, head and neck cancer, hepatocellular carcinoma, cervical cancer, pancreatic cancer, lung cancer, fallopian tube carcinoma, prostate cancer, kidney cancer, bladder cancer, gastrointestinal cancer, melanoma, colorectal cancer, or esophageal cancer.

98. The method of claim 81, further comprising administering to the subject high-dose chemotherapy prior to the administering of the CAR immune cells; or
further comprising administering to the subject bone marrow cells or peripheral blood stem cells; or
further comprising administering to the subject an additional active agent comprising one or more of thalidomide, lenalidomide, and bortezomib.

99.-100. (canceled)

101. The method of claim 81, wherein the effective number of CAR immune cells is approximately 1×10⁴ to approximately 6×10⁵ cells per kg of subject body weight; or
wherein the subject is in a state of minimal residual disease.

102.-103. (canceled)


SEQUENCE LISTING (109 sequences)

SEQ ID NO: 1 - protein, 54 aa, Homo sapiens
MLQMAGQCSQNEYFDSLLHACIPCQLRCSSNTPPLTCQRYCNASVTNSVKGTNA

SEQ ID NO: 2 - protein, 123 aa, synthetic construct
MLQMAGQCSQNEYFDSLLHACIPCQLRCSSNTPPLTCQRYCNASVTNSVKGTNAGGGSGGGSPRGSGGGSMLQMAGQCSQNEYFDSLLHACIPCQLRCSSNTPPLTCQRYCNASVTNSVKGTN

SEQ ID NO: 3 - protein, 272 aa, Homo sapiens
PEEPLVVKVEEGDEAWLPCLKGTSDGPTQQLTWSRESPLKPFLKVSFGVPGLGVHVRPNAVSLVISNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDMTVAPGSTLWLSCGVPPDSVSRGPLSWTHVHPKGPKSLLSLELKDDRPARDMWVTGTRLFLPRATAQDAGKYYCHRGNLTMSFHLEVKARPVSAHTKLRTGGWK

SEQ ID NO: 4 - protein, 272 aa, Homo sapiens
PEEPLVVKVEEGDEAWLPCLKGTSDGPTQQLTWSRESPLKPFLKVSFGVPGLGVHVRPNAVSLVISQVSQQMGGFYLCQPGPPSEKAWQPGWTVNVEGSGELFRWQVSDLGGLGCGLKQRSSEGPSSPSGKLMSPKLYVWAKDRPEIWEGEPPCLPPRDSLQQSLSRDMTVAPGSTLWLSCGVPPDSVSRGPLSWTHVHPKGPKSLLSLELKDDRPARDMWVTGTRLFLPRATAQDAGKYYCHRGQLTMSFHLEVKARPVSAHTKLRTGGWK

SEQ ID NO: 5 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 6 - protein, 47 aa, Homo sapiens
KISHFLKMESLNFIRAHTPYINIYNCEPANPSEKNSPSTQYCYSIQS

SEQ ID NO: 7 - protein, 668 aa, Homo sapiens
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 8 - protein, 53 aa, Homo sapiens
DQWSTQDLYNNPVTAVFNYQGLWRSCVRESSGFTECRGYFTLLGLPAMLQAVR

SEQ ID NO: 9 - protein, 31 aa, Homo sapiens
VTNFWMSTANMYTGMGGMVQTVQTRYTFGAA

SEQ ID NO: 10 - protein, 204 aa, Homo sapiens
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSM

SEQ ID NO: 11 - protein, 147 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 12 - protein, 499 aa, Homo sapiens
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTP

SEQ ID NO: 13 - protein, 248 aa, Homo sapiens
HTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLT

SEQ ID NO: 14 - protein, 258 aa, Homo sapiens
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 15 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 16 - protein, 17 aa, Homo sapiens
IPNGYLVLDLSMQEALS

SEQ ID NO: 17 - protein, 11 aa, Homo sapiens
YNVNDLSMQEL

SEQ ID NO: 18 - skipped

SEQ ID NO: 19 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 20 - protein, 286 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 21 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 22 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 24 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 25 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 26 - protein, 104 aa, synthetic construct
GSHMPKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 27 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 28 - protein, 447 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 428 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQSLSLSPGK

SEQ ID NO: 31 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 444 aa, synthetic construct
EVQLVESGGGLVQPGGSLRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 214 aa, synthetic construct
DIQMTQSPSSLSASVGDRVTITCRTSENIYGGLNWYQRKPGKSPKLLIYGATNLASGVSSRFSGSGSGTDYTLTISSLQPEDVATYYCQNVLRSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 451 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - protein, 214 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 106 aa, synthetic construct
EIVLTQSPGTLSLSPGERATLSCRAQSVSRYLGWYQQKPGQAPRLLIYGASTRATGIPDRFSGSGSGTDFTLTITRLEPEDFAVYYCQQYGSSPWTFGQGTKVEIK

SEQ ID NO: 37 - protein, 118 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSS

SEQ ID NO: 38 - protein, 451 aa, synthetic construct
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 214 aa, synthetic construct
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 447 aa, synthetic construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 214 aa, synthetic construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 107 aa, synthetic construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 43 - protein, 117 aa, synthetic construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 44 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 46 - protein, 450 aa, synthetic construct; identical to SEQ ID NO: 44

SEQ ID NO: 47 - protein, 216 aa, synthetic construct; identical to SEQ ID NO: 45

SEQ ID NO: 48 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 444 aa, synthetic construct
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 51 - protein, 214 aa, synthetic construct
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIK

SEQ ID NO: 53 - protein, 113 aa, synthetic construct
QVQLVESGGGVVQPGRSLRLSCAASGFTFSSYGMHWVRQAPGKGLEWVAVIWYDGSNKYYADSVMGRFTISRDNSKNTLYLQMNSLRAEDTAVYYCASNGDHWGQGTLVTVSS

SEQ ID NO: 54 - protein, 208 aa, Homo sapiens [sequence omitted]

SEQ ID NO: 55 - protein, 224 aa, Homo sapiens
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 56 - protein, 323 aa, Homo sapiens
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 57 - protein, 438 aa, Homo sapiens
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 58 - protein, 121 aa, Homo sapiens
KLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAG

SEQ ID NO: 59 - protein, 312 aa, Homo sapiens
MAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 60 - protein, 250 aa, Homo sapiens [sequence omitted]

SEQ ID NO: 61 - protein, 284 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPI

SEQ ID NO: 62 - protein, 164 aa, Homo sapiens [sequence omitted]

SEQ ID NO: 63 - protein, 329 aa, Homo sapiens
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGG

SEQ ID NO: 64 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 65 - protein, 201 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 66 - protein, 235 aa, Homo sapiens
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKAS

SEQ ID NO: 67 - protein, 121 aa, Homo sapiens
EPKSPKSADKTHTAPQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 69 - protein, 5 aa, synthetic construct
GGGGX

SEQ ID NO: 70 - protein, 6 aa, synthetic construct
GSSGSX

SEQ ID NO: 71 - protein, 5 aa, synthetic construct
GGGGS

SEQ ID NO: 72 - protein, 5 aa, synthetic construct
GSPRG

SEQ ID NO: 73 - protein, 10 aa, synthetic construct
GGGGSGGGGS

SEQ ID NO: 74 - protein, 15 aa, synthetic construct
GGGGSGGGGSGGGGS

SEQ ID NO: 75 - protein, 20 aa, synthetic construct
GGGGSGGGGSGGGGSGGGGS

SEQ ID NO: 76 - protein, 19 aa, synthetic construct
GSPRGGGGSGGGGSGGGGS

SEQ ID NO: 77 - protein, 18 aa, synthetic construct
GSTSGSGKPGSGEGSTKG

SEQ ID NO: 78 - protein, 18 aa, synthetic construct
KESGSVSSEQLAQFRSLD

SEQ ID NO: 79 - protein, 14 aa, synthetic construct
EGKSSGSGSESKST

SEQ ID NO: 80 - protein, 12 aa, synthetic construct
GSAGSAAGSGEF

SEQ ID NO: 81 - protein, 9 aa, Homo sapiens
QSFGLLDPK

SEQ ID NO: 82 - protein, 23 aa, Homo sapiens
LSEGDKVKMDSRIQVLSRGVNQT

SEQ ID NO: 83 - protein, 49 aa, Homo sapiens
KPTTTPAPRPPTPAPTIASQPLSKRPEACRPAAGGAVHTRGLDFACDIY

SEQ ID NO: 84 - protein, 39 aa, Homo sapiens
IEVMYPPPYLDNERSNGTIIHVKGKHLCPSPLFPGPSKP

SEQ ID NO: 85 - protein, 22 aa, Homo sapiens
EPKSCDKTHTCPPCPAPELLGG

SEQ ID NO: 86 - protein, 18 aa, Homo sapiens
ERKCCVECPPCPAPPAAA

SEQ ID NO: 87 - protein, 19 aa, Homo sapiens
ESKYGPPCPPCPAPEFLGG

SEQ ID NO: 88 - protein, 19 aa, Homo sapiens
MKHLWFFLLLVAAPRWVLS

SEQ ID NO: 89 - protein, 18 aa, Homo sapiens
MKWVTFISLLFLFSSAYS

SEQ ID NO: 90 - protein, 19 aa, synthetic construct
MKWVTFISLLFLFSSSSRA

SEQ ID NO: 91 - protein, 21 aa, Homo sapiens
MALPVTALLLPLALLLHAARP

SEQ ID NO: 92 - protein, 16 aa, Homo sapiens
MPLLLLLPLLWAGALA

SEQ ID NO: 93 - protein, 27 aa, Homo sapiens
MGVHECPAWLWLLLSLLSLPLGLPVLG

SEQ ID NO: 94 - protein, 20 aa, Homo sapiens
MYRMQLLSCIALSLALVTNS

SEQ ID NO: 95 - protein, 20 aa, Mus musculus
METDTLLLWVLLLWVPGSTG

SEQ ID NO: 96 - protein, 20 aa, Homo sapiens
MEAPAQLLFLLLLWLPDTTG

SEQ ID NO: 97 - protein, 20 aa, synthetic construct
MEAPAQLLFLLLLWLPSSRA

SEQ ID NO: 98 - protein, 23 aa, Homo sapiens
MDAMKRGLCCVLLLCGAVFVSPS

SEQ ID NO: 99 - protein, 17 aa, Homo sapiens
MLLLLLLLGLRLQLSLG

SEQ ID NO: 100 - protein, 16 aa, synthetic construct
MLLLLLLLLLLALALA

SEQ ID NO: 101 - protein, 21 aa, synthetic construct
MWWRLWWLLLLLLLLWPMVWA

SEQ ID NO: 102 - protein, 133 aa, Homo sapiens [sequence omitted]

SEQ ID NO: 103 - protein, 12 aa, synthetic construct
KDRPEIWEGEPP

SEQ ID NO: 104 - protein, 178 aa, Homo sapiens
PHIQLPPEIQESQEVTLTCLLNFSCYGYPIQLQWLLEGVPMRQAAVTSTSLTIKSVFTRSELKFSPQWSHHGKIVTCQLQDADGKFLSNDTVQPKLEIKVTPSDAIVREGDSVTMTCEVSSSNPEYTTVSWLKDGTSLKKQNTFTLNLREVTKDQSGKYCCQVSNDVGPGRSEEVFLQ

SEQ ID NO: 105 - protein, 85 aa, Homo sapiens
PKLEIKVTPSDAIVREGDSVTMTCEVSSSNPEYTTVSWLKDGTSLKKQNTFTLNLREVTKDQSGKYCCQVSNDVGPGRSEEVFLQ

SEQ ID NO: 106 - protein, 244 aa, Homo sapiens
PKKVTTVIQNPMPIREGDTVTLSCNYNSSNPSVTRYEWKPHGAWEEPSLGVLKIQNVGWDNTTIACAACNSWCSWASPVALNPRDVRVRKIKPLSEIHSGNSVSLQCDFSSSHPKEVQFFWEKNGRLLGKESQLNFDSISPEDAGSYSCWVNNSIGQTASPRRLRVSMSPGDQVMEGKSATLTCESDANPPVSHYTWFDWNNQSLPYHSQKLRLEPVKVQHSGAYWCQGTNSVGKGRSPLSTLT

SEQ ID NO: 107 - protein, 184 aa, Homo sapiens
APRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 108 - protein, 10 aa, synthetic construct
LDSPDRPWNP

SEQ ID NO: 109 - protein, 26 aa, synthetic construct
NQTDKLAAFPEDRSQPGQDCRFRVTQ